(19) United States
(12) Patent Application Publication
(10) Pub. No.: US 2016/0250221 A1
(43) Pub. Date: Sep. 1, 2016

(54) INHIBITORS OF FAPP2 AND USES THEREOF

(71) Applicant: FONDAZIONE TELETHON, Roma (IT)

(72) Inventors: Raffaele LA MONTAGNA, Pozzuoli (NA) (IT); Maria Antonietta DE MATTEIS, Pozzuoli (NA) (IT); Giovanni D'ANGELO, Pozzuoli (NA) (IT)

(21) Appl. No.: 14/907,172

(22) PCT Filed: Jul. 25, 2014
(86) PCT No.: PCT/EP2014/066105

§ 371 Date: Jan. 22, 2016

Related U.S. Application Data
(60) Provisional application No. 61/858,523, filed on Jul. 25, 2013.
(60) Provisional application No. 61/858,516, filed on Jul. 25, 2013.

(51) Int. Cl. A61K 31/5415; A61K 31/192; C12Q 1/68; A61K 31/4045; A61K 31/4453; A61K 31/454; A61K 31/343; A61K 31/41

(52) U.S. Cl. CPC A61K 31/5415; A61K 31/343; A61K 31/192; A61K 31/41; A61K 31/4045; A61K 31/4453; A61K 31/454; C12Q 1/6818

(57) ABSTRACT

The present invention provides methods and compositions for reducing globotriaosylceramide (Gb3) accumulation and treating diseases, disorders or conditions associated with Gb3 accumulation based on inhibitors of phosphatidylinositol-4-phosphate adaptor-2 (FAPP2), including interfering oligonucleotides, for example, siRNAs, and small molecule compounds based inhibitors. The present invention is particularly useful in treating Fabry disease and other sphingolipidoses relating to sphingolipid metabolism, such as Gaucher's disease.

### BACKGROUND

Fabry disease is a glycosphingolipid (GSL) lysosomal storage disorder resulting from an X-linked inherited deficiency of lysosomal α-galactosidase A (α-GAL), an enzyme responsible for the hydrolysis of terminal α-galactosyl residues from glycosphingolipids (Brady et al. N Engl J Med. 1967; 276: 1163-7). A deficiency in α-GAL activity results in a progressive deposition of neutral glycosphingolipids, predominantly globotriaosylceramide (also known as ceramide trihexoside, CD77, Gb3), in the cells of Fabry patients. The accumulation of neutral glycosphingolipids can result in a wide variety of effects, from rash-like developments to stroke and kidney failure.

The frequency of the classical form of disease is estimated to be about 1:40,000 to 1:60,000 in males, and is reported throughout the world within different ethnic groups. Traditional therapy for Fabry disease was enzyme replacement therapy, providing recombinant α-galactosidase A (α-GAL) that is deficient in the Fabry patients. There is still a great medical need for new innovative drugs based on new mechanism of action.

### SUMMARY

The present invention encompasses the discovery that phosphatidylinositol-4-phosphate adaptor-2 (FAPP2) specifically controls the synthesis of globotrioaosylceramide (Gb3), therefore is a novel target for diseases, disorders or conditions associated with Gb3 accumulation. Inhibitors of human FAPP2 can be used to effectively reduce Gb3 accumulation and provide novel therapy for related diseases, disorders and conditions including Fabry disease, and other sphingolipidoses relating to sphingolipid metabolism, such as Gaucher's disease.

As described in the Examples section, the inventors of the present application discovered that GlcCer is channeled by vesicular and non-vesicular transport to two topologically distinct glycosylation tracks in the Golgi cisternae and in the trans golgi network (TGN), respectively. FAPP2 mediates non-vesicular route and delivers GlcCer to the TGN. Surprisingly, FAPP2 depletion selectively inhibited the synthesis of C12-BODIPY-Gb3 but not of C12-BODIPY-GM3, which makes it a novel target for those diseases, disorders, or conditions characterized by Gb3 accumulation. Indeed, the inventors demonstrated that inhibition of FAPP2 (by, e.g., siRNA) decreases Gb3 accumulation in cell models of Fabry disease. The inventors further developed in vitro GlcCer transfer assay to identify inhibitors, in particular, small molecule compounds inhibitors, of FAPP2 and successfully identified for instance phlorizin and other compounds that can inhibit the GlcCer transfer activity of FAPP2 in the in vitro assay. Thus, the present invention provides novel innovative drugs based on new mechanism of action for safer, more effective and affordable treatment of Fabry disease and other diseases, disorders or conditions relating to Gb3 accumulation, or sphingolipid metabolism.

In one aspect, the present invention provides methods of reducing globotrioaosylceramide (Gb3) accumulation in a cell, by administering to a cell having or susceptible to Gb3 accumulation a compound, such as an aryl glucoside compound, that inhibits phosphatidylinositol-4-phosphate adaptor-2 (FAPP2, i.e. a FAPP2 inhibitor). In some embodiments, the compound is an aryl glucoside compound that comprises a glycosidic linkage. In some embodiments, the aryl glucoside compound is a C-aryl glucoside compound. In some embodiments, the aryl glucoside compound is an O-aryl glucoside compound. The aryl glucoside compound may, in some cases, comprise a substituted biaryl group, such as a substituted biphenyl group or a substituted aryl-heteroaryl group (e.g., phenyl-thiophenyl). In some cases, the aryl glucoside compound may comprise a polycyclic aromatic carbocyclic or polycyclic heteroaromatic ring, including and bicyclic aromatic carbocyclic rings and/or bicyclic heteroaromatic rings. In some embodiments, the compound does not comprise a glycosidic linkage.

In some embodiments, the cell is a mammalian cell (e.g., human cell). In some embodiments, the cell is a cultured cell. In some embodiments, the cell is a cell of an organism.

In another aspect, the present invention provides methods of treating a disease, disorder or condition associated with globotrioaosylceramide (Gb3) accumulation, by administering to a subject in need of treatment an aryl glucoside compound that inhibits phosphatidylinositol-4-phosphate adaptor-2 (FAPP2). In some embodiments, the disease, disorder or condition is Fabry disease.

In some embodiments, a suitable aryl glucoside compound has a structure of formula I:

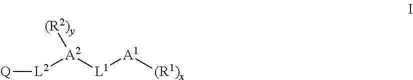

or a pharmaceutically acceptable salt thereof, wherein:
Q is a monosaccharide or modified monosaccharide;
A¹ is phenyl or a 5-6 membered heteroaryl ring having 1-3 heteroatoms independently selected from nitrogen, oxygen and sulfur;
A² is phenyl or a 5-6 membered heteroaryl ring having 1-3 heteroatoms independently selected from nitrogen, oxygen and sulfur;
L¹ is a covalent bond, or a C₁₋₄ bivalent straight or branched hydrocarbon chain, wherein one or two methylene units of the chain are optionally and independently replaced by —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
L² is a covalent bond or —O—;
each R¹ is independently halogen, —CN, —R; —OR; —SR; —N(R)₂; —N(R)C(O)R; —C(O)N(R)₂; —N(R)C(O)N(R)₂; —N(R)C(O)OR; —OC(O)N(R)₂; —N(R)S(O)₂R; —S(O)₂N(R)₂; —OC(O)OR; —C(O)R; —OC(O)R; —C(O)OR; —S(O)R; —S(O)₂R; or Cy;
each R² is independently halogen, —CN, —R, —OR, —SR, —N(R)₂, —N(R)C(O)R, —C(O)N(R)₂, —N(R)C(O)N(R)₂, —N(R)C(O)OR, —OC(O)N(R)₂, —N(R)SO₂R, —S(O)₂N(R)₂, —C(O)R, —C(O)OR, —OC(O)R, —S(O)R, or —S(O)₂R;
Cy is a ring, substituted with p instances of R³; wherein said ring is selected from the group consisting of a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R is independently hydrogen, deuterium, or an optionally substituted group selected from C₁₋₆ aliphatic; a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, —B(OR)₂, or an optionally substituted ring selected from phenyl and 5-6 membered heteroaryl having 1-4 heteroatoms independently selected from nitrogen, oxygen, and sulfur;
p is 1-5;
x is 0-5; and
y is 0-4.

In some embodiments, a suitable aryl glucoside compound has a structure of formula II-a or II-b:

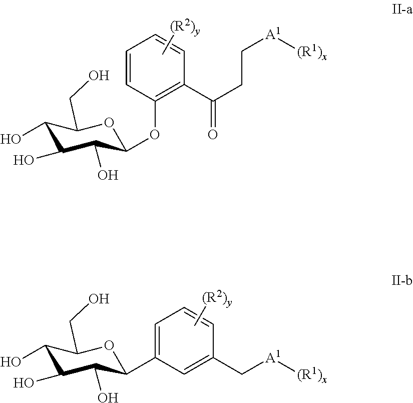

or a pharmaceutically acceptable salt thereof, wherein each of A¹, R¹, R², x, and y is as defined above.

In some embodiments, a suitable aryl glucoside compound has a structure selected from the group consisting of

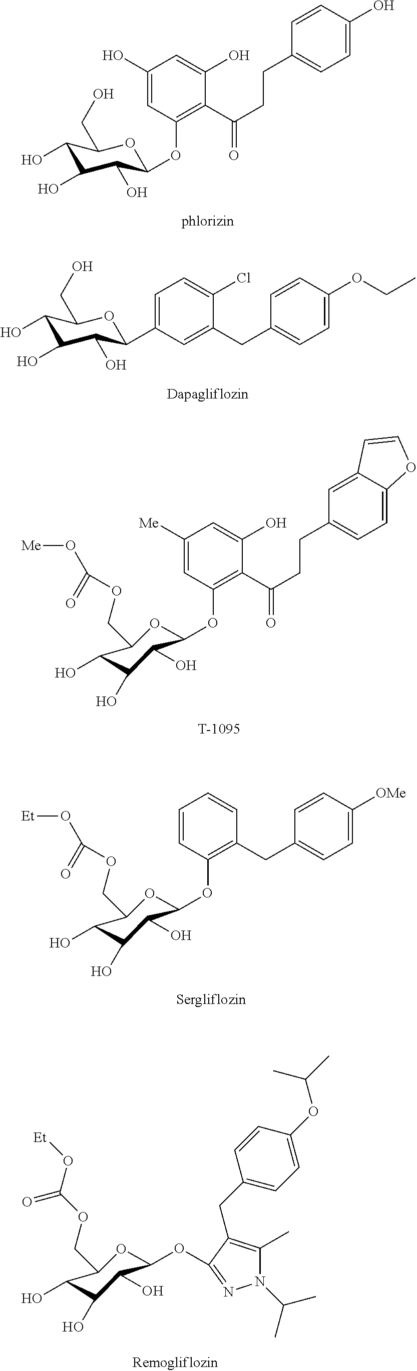

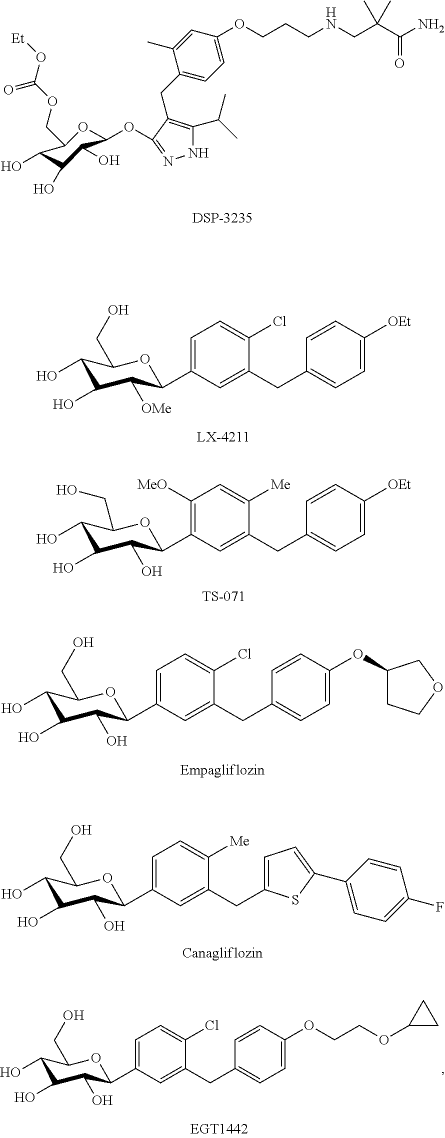

and pharmaceutically acceptable salts thereof.

In some embodiments, a suitable aryl glucoside compound is not Dapagliflozin.

In some embodiments, a suitable aryl glucoside compound has a structure of

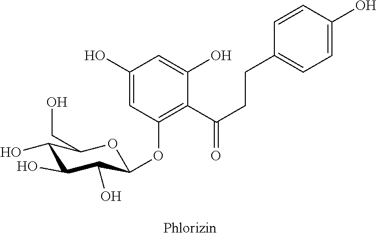

In some embodiments, a suitable aryl glucoside compound has a structure selected from the group consisting of:

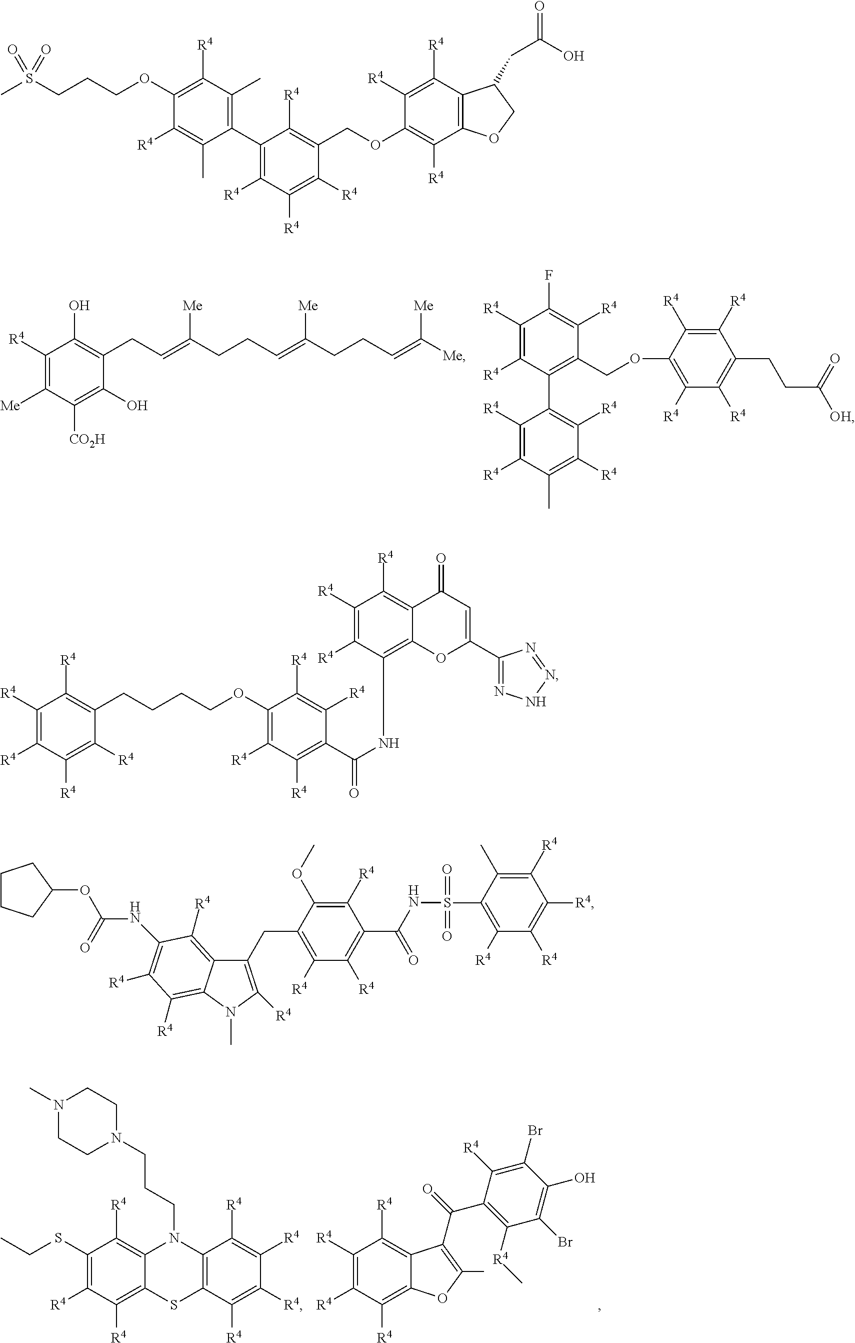

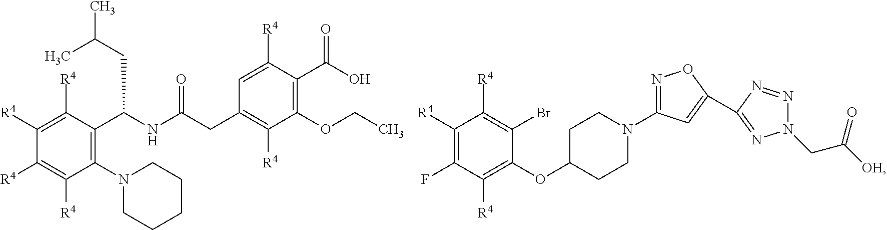

and pharmaceutically acceptable salts thereof,
wherein each R⁴ can be the same or different and is selected from the group consisting of H and -L²-Q, wherein Q is a monosaccharide or modified monosaccharide and L² is a covalent bond or —O—, provided that the aryl glucoside compound includes at least one glycosidic linkage.

In some embodiments, the aryl glucoside compound comprises one glycosidic linkage.

In some embodiments, an inhibitor has a structure selected from the group consisting of:

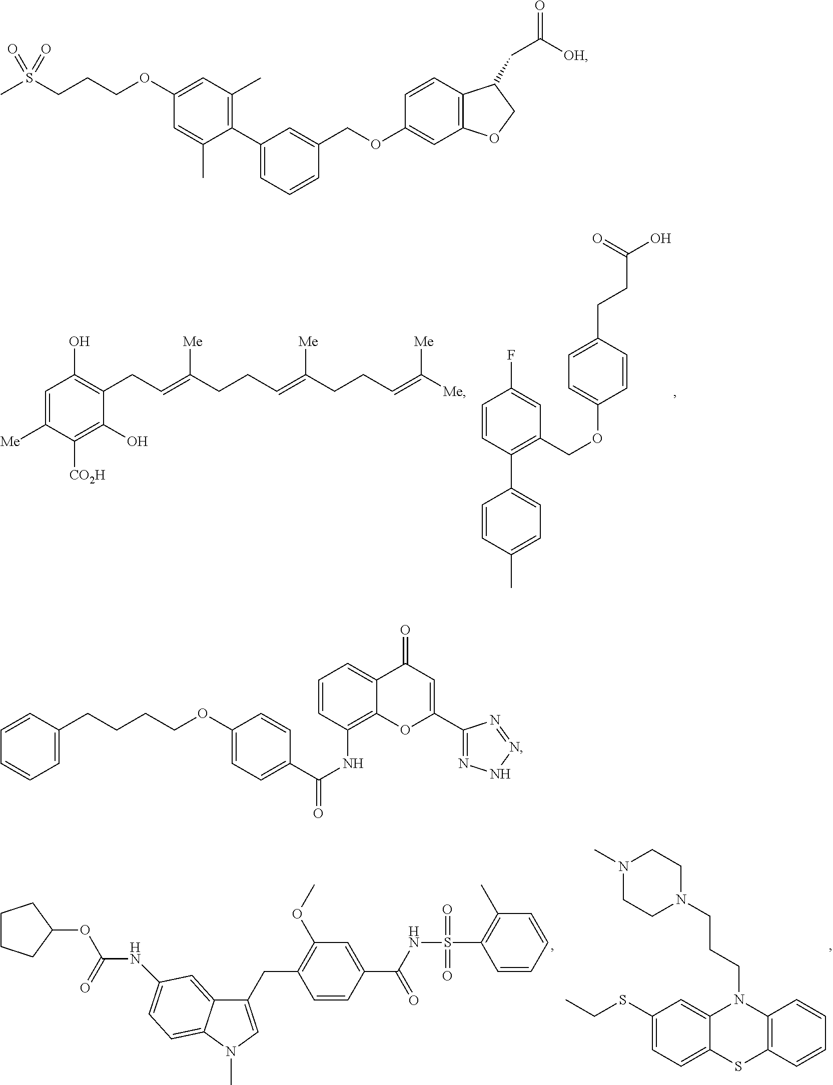

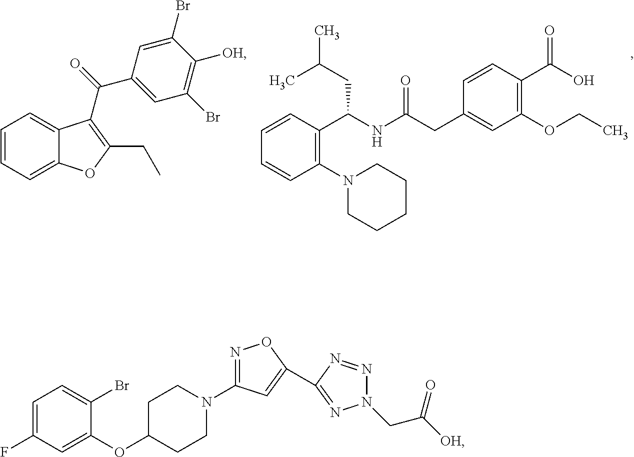 [SMILES: CCc1oc2ccccc2c1C(=O)c1cc(Br)c(O)c(Br)c1 ; CCOc1cc(CC(=O)N[C@@H](CC(C)C)c2ccccc2N2CCCCC2)ccc1C(=O)O ; O=C(O)Cn1nnc(-c2cc(N3CCC(Oc4cc(F)ccc4Br)CC3)no2)n1]

and pharmaceutically acceptable salts thereof.

In yet another aspect, the present invention provides methods of reducing globotrioaosylceramide (Gb3) accumulation in a cell, including administering to a cell having or susceptible to Gb3 accumulation an interfering oligonucleotide that inhibits expression of phosphatidylinositol-4-phosphate adaptor-2 (FAPP2). In some embodiments, an interfering oligonucleotide is an siRNA or shRNA.

In some embodiments, the cell is a mammalian cell (e.g., human cell). In some embodiments, the cell is a cultured cell. In some embodiments, the cell is a cell of an organism.

In still another aspect, the present invention provides methods of treating a disease, disorder or condition associated with globotrioaosylceramide (Gb3) accumulation, including administering to a subject in need of treatment an interfering oligonucleotide that inhibits expression of phosphatidylinositol-4-phosphate adaptor-2 (FAPP2). In some embodiments, an interfering oligonucleotide is an siRNA or shRNA. In some embodiments, the disease, disorder or condition is Fabry disease.

In some embodiments, a suitable interfering oligonucleotide has a sequence that is at least 70% (e.g., at least 75%, 80%, 85%, 90%, 95%, 96%, 97%, 98%, 99%) identical to the reverse complement of a continuous sequence of the human FAPP2 gene or a messenger RNA (mRNA) of FAPP2. In some embodiments, a suitable interfering oligonucleotide has a sequence that is identical to the reverse complement of a continuous sequence of the human FAPP2 gene or a messenger RNA (mRNA) of FAPP2. In some embodiments, the mRNA of FAPP2 comprises FAPP2 mRNA Isoform 1, FAPP2 mRNA Isoform 2, or FAPP2 mRNA Isoform 3.

In some embodiments, a suitable interfering oligonucleotide is or less than 50, 45, 40, 35, 30, 25, 24, 23, 22, 21, 20, 19, 18, 17, 16 or 15 nucleotides in length. In some embodiments, the interfering oligonucleotide is 16-22 (e.g., 16-21, 16-20, 16-19, 16-18, 17-22, 17-21, 17-20, 17-19, 18-22, 18-21, 18-21, or 18-20) nucleotides in length. In some embodiments, the interfering oligonucleotide is an siRNA or shRNA having a sequence selected from

[FAPP2.1]
SEQ ID No. 3
GAGAUAGACUGCAGCAUAU[dT][dT]
[FAPP2.2]
SEQ ID No. 4
GAAUUGAUGUGGGAACUUU[dT][dT]
[FAPP2.3]
SEQ ID No. 5
GAAAUCAACCUGUAAUACU[dT][dT]
[FAPP2.4]
SEQ ID No. 6
CCUAAGAAAUCCAACAGAA[dT][dT]
[sh FAPP2.1]
SEQ ID No. 7
CTCTTGTGGCTGAAGAGAGGTCTCAAATT;
[shFAPP2.2]
SEQ ID No. 8
TTGGCAGCCTCGATGGTTCCTTCTCTGTG;
[shFAPP2.3]-
SEQ ID No. 9
CAGTCTGGATCAGACTCAAGTTGCTCTCC;
and/or
[shFAPP2.4]
SEQ ID No. 10
TCCTGTTAAGATGGATCTTGTTGGAAATA.

In some embodiments, a suitable interfering oligonucleotide contains at least one chemical modification. In some embodiments, the at least one chemical modification is selected from the group consisting of conformationary constraint nucleotide analogue (e.g., locked nucleic acid), 2′O-methyl modification, phosphorothioate linkage, and combination thereof.

The present invention also provides pharmaceutical composition comprising a phosphatidylinositol-4-phosphate adaptor-2 (FAPP2) inhibitor as defined in any of claims 1 to 27 and a pharmaceutically acceptable carrier for use in a method of reducing globotrioaosylceramide (Gb3) accumulation in a cell having or susceptible to Gb3 accumulation or for use for the prevention and/or treatment of a disease, disorder or condition characterized by globotrioaosylceramide (Gb3) accumulation.

The present invention also provides a method to identify a phosphatidylinositol-4-phosphate adaptor-2 (FAPP2) inhibitor comprising:
mixing acceptor vesicles, donor vesicles containing a fluorescent-labeled moiety, a quencher, and recombinant FAPP2 protein to form a mixture; and
measuring the emission intensity of the mixture either in the presence or absence of an agent, wherein if the emission intensity is decreased in the presence of the agent, said agent is identified as a FAPP2 inhibitor.

In some embodiments, the method comprises:
mixing acceptor vesicles containing 1,2-dioleoyl-sn-glycero-3-phosphocholine (DOPC) with donor vesicles containing TopFLUOR-labeled GlcCer (preferably 1 mole %) and Di1C18 (preferably 3 mole %), and recombinant FAPP2 protein (preferably 0.5 uM) to form a mixture; and
measuring the emission intensity of the mixture at 520 nm (excitation at 485 nm) in the presence or absence of an agent, wherein if the emission intensity is decreased in the presence of the agent, said agent is identified as a FAPP2 inhibitor.

In some embodiments in the method the recombinant FAPP2 protein is FAPP2-GLTP-C212 or FAPP2 Full-Length (FL). In some embodiments, the acceptor vesicles are formed by sonication of 1,2-dioleoyl-sn-glycero-3-phosphocholine (DOPC) suspended in buffer.

FAPP2 transfer activity has been evaluated using Fluorescence Resonance Energy Transfer. The FRET assay involves mixing of acceptor vesicles (formed by sonication of 1,2-dioleoyl-sn-glycero-3-phosphocholine (DOPC) suspended in buffer) with donor vesicles containing TopFLUOR-labeled GlcCer (1 mole %) and Di1C18 used as quencher (3 mole %), and recombinant FAPP2 protein (0.5 uM). Recovery of emission intensity at 520 nm (excitation at 485 nm) occurs during FAPP2-mediated transfer of GlcosylCeramide from quenched donor vesicles to unquenched acceptor vesicles. The assay has been performed using FAPP2-GLTP-C212 or FAPP2 Full-Length (FL). In order to conduct the high throughput inhibitor screen, the transfer activity assay was adapted to a microplate format (384 well plate) and read using Synergy Neo HTS Multi-Mode Microplate Reader. First, mixture containing 30 ul of acceptor small unilamellar vesicles, FAPP2 transport protein, and drug in buffer was added to each well in triplicate and read for 1 min to calculate the fluorescence baseline. Then 30 ul donor vesicles were added to each well and read for 15 or 30 mins. Since the increase in fluorescence emission occurs exclusively in presence of FAPP2 transport, the inhibition rate of each compound is evaluated by its ability to decrease the fluorescence emission.

Among other things, the present invention also provides pharmaceutical compositions or kits including one or more small molecules or interfering oligonucleotides described herein and a pharmaceutically acceptable carrier.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The drawings are for illustration purposes only, not for limitation.

FIG. 1A depicts an exemplary schematic of a GSL synthetic pathway in vertebrates. Abbreviations are as follows: SM, sphingomyelin; GCS, GlcCer synthase; LCS, LacCer synthase; Gb3S, Gb3 synthase; GM3S, GM3 synthase; LC3S, LC3 synthase; GA2S, GA2 synthase.

FIG. 1B depicts exemplary expression of FAPP2 in different mouse tissues.

FIG. 1C depicts an exemplary southern blot analysis of wild-type (FAPP2^{+/+}) and recombinant (FAPP2^{geo/+}) and (FAPP2^{geo/geo}) embryonic stem cells.

FIG. 1D depicts exemplary FAPP2 levels in FAPP2+/+ and FAPP2−/− testis and kidney extracts.

FIG. 1E and FIG. 1F depict exemplary results of cholera toxin B fragment (ChTxB), Shiga toxin B fragment (ShTxB), and Anti-GM3 staining of FAPP2+/+ and FAPP2−/− kidney cortex sections. Exemplary pictures are from at least five FAPP2+/+ and five FAPP2−/− mice. Bars: 50 μm.

FIG. 2A depicts an exemplary HPTLC profile of ³H-sphingosine-labelled HeLa cells. Arrows: changes induced by FAPP2 KD knock down; numbers: percentage of each GSL species on total SLs (sphingolipids).

FIG. 2B depicts an exemplary HPTLC profile of C12-BODIPY-GlcCer labelled HeLa cells. Arrows: GSLs reduced by FAPP2-KD; numbers: percentage of each GSL species on total GSL; #: unassigned peak.

FIG. 2C depicts exemplary results of silencing FAPP2 and GSL synthetic enzymes (GCS, GlcCer synthase, LCS, LacCer synthase, GM3S, GM3 synthase, Gb3S, Gb3 synthase) on different GSL species (expressed as percentage of total GSLs); numbers: percentage of total GSLs on total SL. Results are means±SD of at least three independent experiments.

FIG. 3A depicts an exemplary effect of Brefeldin A (BFA) (5 μg/mL) on Gb3 and GM3 synthesis.

FIG. 3B depicts an exemplary distribution of HA-Gb3S and HA-GM3S by immunofluorescence. Upper panels: untreated cells, lower panels: nocodazole-treated cells (3 hours 33 μM). Insets: enlargement of the boxed areas. The colocalization of HA-Gb3S and HA-GM3S with TGN46 is 50% and 14%, respectively. Data are representative of at least 30 cells/condition. Bar, 10 μm.

FIG. 3C depicts an exemplary distribution of HA-Gb3S and HA-GM3S by immunoEM. Arrows: clathrin-coated profiles at the trans Golgi. Data are representative of at least 30 stacks. Bar, 100 nm.

FIG. 3D depicts an exemplary effect of intra-Golgi trafficking blockage on the transport of a reporter protein (the glycoprotein of the vesicular stomatitis virus (VSVG) (means±SD in three independent experiments for at least 100 cells/time point). Means±SD of three independent experiments. DIC: dicoumarol (200 μM). knock down of PLA2 (PLA2-KD) or knock down of Bet3 (BET-3 KD).

FIG. 3E depicts an exemplary effect of intra-Golgi trafficking blockage on GM3 and Gb3 synthesis (3 hours ³H-sphingosine pulse). Means±SD of three independent experiments. DIC: dicoumarol (200 μM), PLA2-KD: knock down of PLA2, BET-3 KD: knock down of Bet3.

FIG. 4A depicts an exemplary sub-golgi distribution of FAPP2-wt and FAPP2 W407A in nocodazole-treated cells (3 hours, 33 μM). Right panels: enlargement of boxed areas and distribution of the maximal fluorescence intensity of FAPP2 along the cis-Golgi-to-TGN axis (white arrows).

FIG. 4B depicts an exemplary quantification of the maximal labelling distribution of FAPP2-wt and FAPP2-W407A. The middle of the stack (0, black dashed line) is taken as a plane equidistant from GM130 and TGN46 fluorescent intensity peaks in at least 50 stacks per condition. Bar, 10 μm.

FIG. 4C depicts the intra-Golgi distribution of FAPP2-wt and FAPP2-W407A in Meb4 and GlcCer-deficient GM95 cells. The percentage of labelling associated with the TGN is indicated. Means±SEM of at least 30 stacks/condition. Arrowheads: Golgi cisternae staining; wedges: TGN staining, arrows: clathrin-coated profiles. Bar, 100 nm.

FIG. 4D depicts an exemplary schematic of intra-Golgi non-vesicular (red arrow) and vesicular (blue arrow) transport of GlcCer. Inset: mechanism of FAPP2-mediated GlcCer-transfer directionality (cyan profiles: TGN, red profiles: Golgi cisternae).

FIG. 5 illustrates an exemplary result validating FAPP2 as a target in fibroblasts from Fabry disease (FD) patients: FAPP2 KD decreases Gb3 accumulation in FD fibroblasts. Fibroblasts from six different FD patients (mutations described in Example 1) were left untreated or treated with siRNA specific for FAPP2 (table 2) for 72 hrs and then processed for immunofluorescence and stained for Gb3 with Cy3-Shiga toxin fragment b (red) and for a lysososmal marker (LAMP1, green). Mock (treated with transfection vehicle), FAPP2-KD (treated with siRNA specific for FAPP2), LAMP1 (late endosome/lysosome marker), SHIGA TOXIN (Gb3 marker).

FIG. 6A illustrates an exemplary result of HeLa cells treated with siRNA-GLA

siRNA GLA#1
SEQ ID No. 90
GCUAUCAUGGCUGCUCCUU
siRNA GLA#2
SEQ ID No. 91
GCAAUCACUGGCGAAAUUU
siRNA GLA#3
SEQ ID No. 92
CAGCUUAGACAGGGAGACA

and analyzed with the Operetta. HeLa cells were transfected in suspension with siRNA-GLA and plated in a 96-well plate. 72 hours later cells were fixed in 4% PFA, permeabilized with saponin-containing blocking buffer, and stained with a fluorescent recombinant Shiga toxin B (that specifically binds Gb3), an antibody against LampI, and Hoechst 33342. Images were captured using the Operetta. To obtain double KD, HeLa cells were incubated for 72 hours with a mix of siRNA against FAPP2 and GLA. Cells were then processed for immunofluorescence, using the same protocol described before.

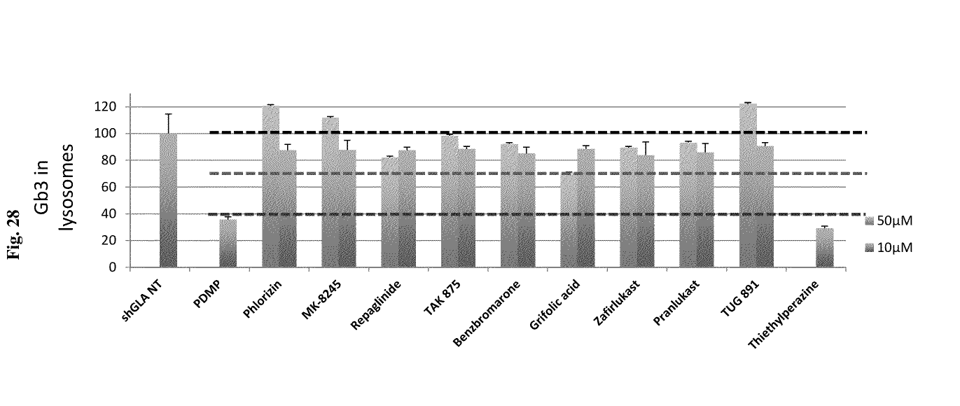

FIG. 6B depicts an exemplary quantitative analysis of the intensity of the Gb3 staining obtained in FIG. 6A.

FIG. 7 depicts an exemplary effect of Phlorizin inhibition on the GlcCer transfer activity of FAPP2. RFU (Relative Fluorescence Unit), FAPP2-FL-SUMO (recombinant FAPP2 full length protein tagged with small ubiquitin-related modifier)

FIG. 7A depicts an exemplary result demonstrating FAPP2 transfers GlcCer from donor to acceptor liposomes in concentration-dependent fashion.

FIG. 7B depicts an exemplary result illustrating GlcCer (C8-G1Cer), but not ceramide (C6-ceramide) competes with the GlcCer transfer activity of FAPP2. The final concentration of both C8-GlcCer and C6-Cer was 10 uM.

FIG. 7C depicts an exemplary result illustrating Phlorizin inhibits the GlcCer transfer by FAPP2. The assay was conducted at four different drug concentrations (100 uM, 200 uM, 500 uM, 1 mM).

FIG. 7D depicts an exemplary result illustrating Dapagliflozin has no inhibitory activity on GlcCer transfer activity of FAPP2. The assay was conducted at four different drug concentrations (100 uM, 200 uM, 500 uM, 1 mM). Dapagliflozin administration did not inhibit the transfer GlcCer transfer, yet administration induced an increase in fluorescence.

FIG. 8A depicts exemplary restriction maps of wild-type FAPP2 allele (+), targeting vector, targeted allele (geo), floxed allele obtained by crossing with Flp transgenic mice (flox), and the null FAPP2 allele (−) obtained after Cre-mediated excision of exon 4 (see Example 1).

FIG. 8B depicts exemplary distribution of FAPP2 as assessed by X-Gal and haematoxylin-eosin (HE) staining in the indicated tissues from FAPP2geo/geo 8-to-10-week-old mice. Bars, 100 μm.

FIG. 9A depicts exemplary immunohistochemistry of FAPP2 and GM130 expression in isolated kidney tubular cells from wt and FAPP2−/− mice. Cells are stained with anti-GM130 antibodies (green) and anti-FAPP2 (red) antibodies. Kidney tubular cells were isolated according to the procedure described in ³⁵.

FIG. 9B depicts an exemplary FAPP2 Western Blot analysis of lysates from Kidney cells.

FIG. 9C depicts an exemplary FACS analysis of isolated tubular kidney cells double stained with fluorescently labelled. ChTxB (CholeraToxin fragment B, GM1 marker (green) and ShTxB (Shiga Toxin fragment B, Gb3 marker (red) (upper panels). Dotted lines indicate threshold values for background staining The lower panels show the frequency of ChTxB- or ShTxB-positive cells. The arrow indicates the selective reduction in the frequency of ShTxB positive cells.

FIG. 9D depicts exemplary immunofluorescence of isolated tubular kidney cells double stained as described in FIG. 9C. Bars; 10 μm.

FIG. 10 depicts an exemplary result of protein down-regulation after siRNA treatments. The proteins FAPP2, Bet3, and cPLA2 were detected using specific antibodies in the indicated mock- or siRNA-treated (KD) cells siRNA FAPP2 (Table 2) and siRNA Bet3 (Table3) (HeLa, MDCK, HK2, HepG2, SK-N-MC). Actin was taken as an internal control protein. The sequences of the different siRNAs are described in Table 3 (see Example 2). In all the experiments, the interference has been performed using a pool of the different siRNA sequences (reported in Tables 2 and 3).

FIG. 11 depicts an exemplary result showing FAPP2 selectively controls Gb3 synthesis.

FIG. 11A depicts an exemplary pulse-chase-HPTLC analysis of mock-treated or FAPP2-KD HeLa cells, pulsed with ³H-sphingosine for 2 hours and chased for 0, 2, 6, and 24 h. Results are the means of at least 3 independent experiments±SEM.

FIG. 11B depicts an exemplary result comparing the effects on GSL levels induced by silencing the different GSL synthetic enzymes or FAPP2 with an RT-qPCR-based assessment of siRNA-mediated silencing of genes involved in GSL synthesis (see Tables 3 and 4). GCS, GlcCer synthase; LCS, LacCer synthase; GM3S, GM3 synthase; Gb3S, Gb3 synthase.

FIG. 11C depicts an exemplary result comparing effects of siRNA-mediated silencing of the indicated genes on GSL synthesis assessed in cells labelled with C12-BODIPY-GlcCer for 3 hours. Numbers indicate the percentage of C12-BODIPY-GlcCer incorporated into each given GSL compared to the total GSLs.

FIG. 11D depicts an exemplary result, wherein a HeLa cell population expressing both Gb3 and GM1 at levels detectable by ShTxB and ChTxB, respectively, was selected by FACS (mock) and then subjected to treatment with FAPP2 siRNA (FAPP2-KD). The blue box delimits values of ShTxB and ChTxB staining corresponding to background staining. Numbers indicate the percentage of double-positive cells. The decrease in this percentage induced by FAPP-KD is statistically significant (p<0.001) and is paralleled by the increase in the percentage of cells that express only GM1 (from 16% to 28%).

FIG. 12A depicts an exemplary mathematical modeling of GSL metabolic fluxes in control and FAPP2 KD cells. Reactions and reaction rates considered in the mathematical modelling of the experimental data shown in FIG. 11A. k=reaction rates (k1-k5).

FIG. 12B depicts exemplary optimized reaction rates and Cost Functions (CF) under different simulation conditions in which the reaction rates were either required to be all equal (null hypothesis, N) or were allowed to vary one at a time (red boxed cells) between mock-treated (blue) and FAPP2 KD cells (red). The reaction rates extracted from the simulation leading to the lowest CF are indicated in bold and were used for the exemplary metabolic model shown in FIG. 12C. N (null hypothesis). In the initial simulation, all reaction rates were required to have the same value for mock-treated and FAPP2-KD cells.

FIG. 12C depicts an exemplary metabolic model, wherein dotted lines refer to experimental data and continuous lines represent the best fit obtained from mathematical modelling (see Example 1).

FIG. 13 depicts an exemplary effect of BFA on 3XHA-Gb3S and 3XHA-GM3S distribution.

FIG. 13A depicts an exemplary result of immunofluorescence showing localization of GM3S and Gb3S at steady state (CTRL) and upon BFA treatment (5 μg/ml 30 min) (BFA).

FIG. 13B depicts an exemplary result of immunofluorescence showing Gb3S, TGN46 and their co-localization after BFA treatment (merge). Bars; 10 μm.

FIG. 14A depicts an exemplary result illustrating the effect of FAPP2-KD on GSL synthesis in different cell lines (HeLa, MDCK, HepG2, HK2). Also shown (last bar graph) are the GSLs synthesized in mouse embryo fibroblasts (MEF) from wt and FAPP2−/− mice. GSL synthesis was assessed by “C-galactose (HeLa, MDCK, HepG2) or ³H-sphingosine (HK2, MEF) labelling (6 hours). Asterisks indicate statistically significant differences with control or untreated cells. * p<0,05; ** p<0,01; *** p<0.001.

FIG. 14B depicts an exemplary result illustrating the effect of BFA on GSL synthesis in the indicated cell lines (HeLa, MDCK, HepG2, HK2 cells). GSL synthesis was assessed by ³H-sphingosine or “C-galactose (MDCK) labelling (3 hours). Asterisks indicate statistically significant differences with control or untreated cells. * p<0,05; ** p<0,01; *** p<0.001.

FIG. 15A depicts an exemplary graph showing the efficiency of B4GALT5 and B4GALT6 KD following specific siRNA treatment as estimated by RT-qPCR.

FIG. 15B depicts an exemplary result illustrating the effect of B4GALT5 KD and B4GALT6 KD on sphingolipid levels in HeLa cells labelled with ³H-sphingosine for 24 hours.

FIG. 15C depicts an exemplary result illustrating localization of B4GALT5 assessed by immunofluorescence in comparison with a TGN marker (TGN46).

FIG. 15D depicts an exemplary result illustrating localization of B4GALT5 assessed by IEM. Black arrowheads indicate B4GALT5 localized in the Golgi, wedges indicate B4GALT5 localized in the TGN; the black arrow points to a clathrin-coated round profile, indicative of the TGN. Bars (c)=10 μm; (d)=100 nm.

FIG. 16A depicts an exemplary result illustrating that ectopic expression of GM3S at the TGN renders GM3 synthesis sensitive to FAPP2 depletion. Localization of 3XHA-GM3S in cells expressing different amounts of the protein. IEM of Golgi stacks from cells expressing low levels (lower panel) or high levels (upper panel) of 3XHA-GM3S. Arrowheads point to TGN-localized staining.

FIG. 16B depicts an exemplary quantitative analysis of the TGN localization of 3XHA-GM3S in relation to the levels of expression.

FIG. 16C depicts an exemplary result illustrating sphingolipid synthesis in HeLa cells overexpressing GM3S (HeLa-GM3S) in comparison to parental HeLa cells as assessed by a 3 h pulse with ³H-sphingosine.

FIG. 16D depicts an exemplary result illustrating the effect of BFA treatment (5 μg/mL) on HeLa cells overexpressing GM3S (HeLa-GM3S) in comparison to parental HeLa cells. Values are expressed as percent of control (CTRL) taken as untreated parental HeLa cells.

FIG. 16E depicts an exemplary result illustrating the effect of FAPP2-KD on GM3 synthesis (3 hours ³H-sphingosine pulse) in HeLa cells overexpressing GM3S (HeLa-GM3S) compared to parental HeLa cells. Values are expressed as percent of control (CTRL) taken as mock-treated parental HeLa cells.

FIG. 17A depicts an exemplary result illustrating that the E50A mutant of the FAPP2-PH domain does not stabilize ARF1 on the Golgi complex. Cos7 cells transfected with plasmids encoding GFP-tagged diFAPP2-PH wt or E50A, a mutant in the ARF1 binding site¹⁹, were processed for indirect immunofluorescence with anti-ARF1 antibodies. Asterisks indicate transfected cells. Bar, 10 μm.

FIG. 17B depicts exemplary quantification of the stabilization of ARF1 on the Golgi complex evaluated as percentage of Golgi-associated ARF1-fluorescence to total ARF1 fluorescence.

FIG. 17C depicts an exemplary result illustrating tandem PH domains of FAPP2 in the wt form (diPH wt, which can bind both ARF and PtdIns4P) or in the E50A mutant form (diPH-E50A, which cannot bind ARF, see above FIG. 17A and ¹⁹), or in the R18L form (diPHR18L, which cannot bind PtdIns4P ¹⁴), expressed as GFP chimerae and their intra-Golgi distribution analyzed by immunoelectron microscopy (Bar, 100 nm). Black arrows point to clathrin-coated profiles, which are indicative of the TGN. Right panel shows the quantification of TGN- and cisternae-associated particles. Data are means±S.E.M. of at least 30 stacks analyzed per condition.

FIG. 18A depicts an exemplary result of GlcCer loading of FAPP2. GlcCer induces a shift of the tryptophan fluorescence in FAPP2; cyan lines indicate tryptophan fluorescence at increasing concentrations of C8-GlcCer (from 0 to 1.2 μM, as detailed in the inset); the arrow indicates the change in tryptophan fluorescence maximal emission; the inset shows the effect of increasing concentrations of C8-GlcCer on tryptophan maximal emission.

FIG. 18B depicts an exemplary result illustrating the effect of C8-GlcCer loading on recombinant FAPP2-wt and FAPP2-W407A circular dichroism.

FIG. 18C depicts an exemplary result illustrating the effect C8-GlcCer loading on

FAPP2 affinity for POPC or POPC and PtdIns4P-containing liposomes as measured by Surface Plasmon Resonance. Increasing concentrations of FAPP2 (ranging from 0.5 to 1.5 mg/mL) in its apo-form or loaded with equimolar amount of C8-GlcCer, were used. Results are representative of at least three independent experiments.

FIG. 19A depicts TAK-875 dose response assay using FAPP2-HIS-SUMO-C-212 at 0.5 uM.TAK-875 activity was assessed using Fluorescence resonance energy transfer assay. The assay was conducted at three different drug concentrations (100 uM, 50 uM, 25 uM) and FAPP2 at 0.5 uM. Inhibition of FAPP2 activity by TAK-875 was measured for 15 mins. 100 uM TAK-875 significantly reduced FAPP2-mediated GlcCer transfer.

FIG. 19B shows the inhibition rate of 100 uM TAK-875 on FAPP2 velocity transfer at time zero.

FIG. 20A depicts grifolic acid dose response assay using FAPP2-HIS-SUMO-C-212 at 0.5 uM. Grifolic Acid activity was assessed using Fluorescence resonance energy transfer assay. The assay was conducted at four different drug concentrations (100 uM, 50 uM, 10 uM, 1 uM) and FAPP2-C212 at 0.5 uM. Inhibition of FAPP2 activity by Grifolic Acid was measured for 30 mins. 100 uM and 50 uM Grifolic Acid significantly reduced FAPP2-mediated GlcCer transfer

FIG. 20B shows the inhibition rate of 50 uM of Grifolic Acid on FAPP2 velocity transfer at time zero.

FIG. 21A depicts TUG-891 dose response assay using FAPP2-HIS-SUMO-C-212 at 0.5 uM. TUG 891 activity was assessed using Fluorescence resonance energy transfer assay. The assay was conducted at four different drug concentrations (100 uM, 50 uM, 10 uM, 1 uM) and FAPP2-C212 at 0.5 uM. Inhibition of FAPP2 activity by TUG 891 was measured for 30 mins. 50 uM TUG-891 inhibits 50% FAPP2 transfer activity.

FIG. 21B shows the inhibition rate of 50 uM TUG-891 on FAPP2 velocity transfer at time zero.

FIG. 22A depicts pranlukast dose response assay using FAPP2-HIS-SUMO-C-212 at 0.5 uM. Pranlukast activity was assessed using Fluorescence resonance energy transfer assay. The assay was conducted at four different drug concentrations (100 uM, 50 uM, 10 uM, 1 uM) and FAPP2-C212 at 0.5 uM. Inhibition of FAPP2 activity by Pranlukast was measured for 30 mins 50 uM Pranlukast inhibits 90% FAPP2 transfer activity.

FIG. 22B shows the inhibition rate of 50 uM Pranlukast on FAPP2 velocity transfer at time zero.

FIG. 23A depicts Zafirlukast dose response assay using FAPP2-HIS-SUMO-C-212 at 0.5 uM. Zafirlukast activity was assessed using Fluorescence resonance energy transfer assay. The assay was conducted at four different drug concentrations (100 uM, 50 uM, 10 uM, 1 uM) and FAPP2-C212 at 0.5 uM. Inhibition of FAPP2 activity by Zafirlukast was measured for 30 mins. 50 uM Zafirlukast inhibits 90% FAPP2 transfer activity.

FIG. 23B shows the inhibition rate of 50 uM Zafirlukast on FAPP2 velocity transfer at time zero.

FIG. 24A depicts thiethylperazine dose response assay using FAPP2-HIS-SUMO-C-212 at 0.5 uM. Thiethylperazine activity was assessed using Fluorescence resonance energy transfer assay. The assay was conducted at four different drug concentrations (100 uM, 50 uM, 10 uM, 1 uM) and FAPP2-C212 at 0.5 uM Inhibition of FAPP2 activity by Thiethylperazine was measured for 30 mins 50 uM Thiethylperazine inhibits 60% FAPP2 transfer activity.

FIG. 24B shows the inhibition rate of 50 uM Thiethylperazine on FAPP2 velocity transfer at time zero.

FIG. 25A depicts benzbromarone dose response assay using FAPP2-HIS-SUMO-C-212 at 0.5 uM. Benzbromarone activity was assessed using Fluorescence resonance energy transfer assay. The assay was conducted at four different drug concentrations (100 uM, 50 uM, 10 uM, 1 uM) and FAPP2-C212 at 0.5 uM Inhibition of FAPP2 activity by Benzbromarone was measured for 30 mins 50 uM Benzbromarone inhibits 80% FAPP2 transfer activity.

FIG. 25B shows the inhibition rate of 50 uM Benzbromarone on FAPP2 velocity transfer at time zero.

FIG. 26A depicts Repaglinide dose response assay using FAPP2-FL-SUMO-HIS at 0.5 uM. Repaglinide activity was assessed using Fluorescence resonance energy transfer assay. The assay was conducted at different drug concentrations (100 uM, 50 uM, 25 uM) and FAPP2 FL-SUMO-HIS at 0.5 uM. Inhibition of FAPP2 activity by Repaglinide was measured for 15 mins. 50 uM Repaglinide inhibits 50% FAPP2 transfer activity.

FIG. 26B shows the inhibition rate of 50 uM Repaglinide on FAPP2-FL velocity transfer at time zero.

FIG. 27A depicts MK-8245 dose response assay using FAPP2-FL-SUMO-HIS at 0.5 uM. MK-8245 activity was assessed using Fluorescence resonance energy transfer assay. The assay was conducted at different drug concentrations (100 uM, 50 uM, 25 uM) and FAPP2-FL-SUMO-HIS at 0.5 uM. Inhibition of FAPP2 activity by MK-8245 was measured for 15 mins 50 uM MK-8245 inhibits 40% FAPP2 transfer activity.

FIG. 27B shows the inhibition rate of 50 uM MK-8245 on FAPP2 velocity transfer at time zero.

FIG. 28 shows the effect of ten compounds selected as inhibitors of GlcCer transfer activity of FAPP2 on the accumulation of Gb3 within lysosomes. Each histogram represents a percentage of intensity of Gb3 in lysosomes respect to the negative control (shGLA NT), which is expressed as 100%. PDMP treatment has been used as positive control (10 μM). Hits have been tested at 10 μM (red blocks) and 50 μM (blue blocks). Dashed lines indicate levels of Gb3 accumulation in negative control (black line), positive control (red line), and in median condition between controls (green line).

### DEFINITIONS

In order for the present invention to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Amelioration: As used herein, the term “amelioration” is meant the prevention, reduction or palliation of a state, or improvement of the state of a subject. Amelioration includes, but does not require complete recovery or complete prevention of a disease condition.

Dysfunction: As used herein, the term “dysfunction” refers to an abnormal function. Dysfunction of a molecule (e.g., a protein) can be caused by an increase or decrease of an activity associated with such molecule. Dysfunction of a molecule can be caused by defects associated with the molecule itself or other molecules that directly or indirectly interact with or regulate the molecule.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of disease as the individual being treated, who is about the same age as the individual being treated (to ensure that the stages of the disease in the treated individual and the control individual(s) are comparable).

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal.

Modulator: As used herein, the term “modulator” refers to a compound that alters or elicits an activity. For example, the presence of a modulator may result in an increase or decrease in the magnitude of a certain activity compared to the magnitude of the activity in the absence of the modulator. In certain embodiments, a modulator is an inhibitor, which decreases the magnitude of one or more activities. In certain embodiments, an inhibitor completely prevents one or more biological activities. In certain embodiments, a modulator is an activator, which increases the magnitude of at least one activity. In certain embodiments the presence of a modulator results in a activity that does not occur in the absence of the modulator.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention may be specifically directed to “unmodified nucleic acids,” meaning nucleic acids (e.g. polynucleotides and residues, including nucleotides and/or nucleosides) that have not been chemically modified in order to facilitate or achieve delivery.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Small molecule: In general, a “small molecule” is understood in the art to be an organic molecule that is less than about 5 kilodaltons (Kd) in size. In some embodiments, the small molecule is less than about 4 Kd, about 3 Kd, about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Subject: As used herein, the term “subject” refers to a human or any non-human animal (e.g., mouse, rat, rabbit, dog, cat, cattle, swine, sheep, horse or primate). In many embodiments, a subject is a human being. A human includes pre and post natal forms. In certain embodiments of the present invention the subject is an adult, an adolescent or an infant. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder. Also contemplated by the present invention are the administration of the pharmaceutical compositions and/or performance of the methods of treatment in-utero.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains Substitution of one amino acid for another of the same type may often be considered a “homologous” substitution.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” refers to an amount of a therapeutic agent which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific agent employed; the duration of the treatment; and like factors as is well known in the medical arts.

Treatment: As used herein, the term “treatment” (also “treat” or “treating”) refers to any administration of a therapeutic agent (e.g., oligonucleotide, small molecule) that partially or completely alleviates, ameliorates, relieves, inhibits, delays onset of, reduces severity of and/or reduces incidence of one or more symptoms or features of a particular disease, disorder, and/or condition (e.g., Fabry disease). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

A phosphatidylinositol-4-phosphate adaptor-2 (FAPP2) inhibitor: is a compound able to inhibit the GlucosylCeramide transfer activity of FAPP2)

A disease, disorder or condition characterized by globotrioaosylceramide (Gb3) accumulation is for instance Fabry disease wherein a deficiency of the enzyme alpha-galactosidase results in the accumulation of Gb3 within lysosomes. This leads to abnormal function of many cells and blood vessels throughout the body. This dysfunction affects many of the organs and body systems; such as Kidney, Heart, Nervous System, Eyes, Skin, and Gastointestinal tract.

In the present invention “an interfering oligonucleotide that inhibits expression of phosphatidylinositol-4-phosphate adaptor-2 (FAPP2)” may be identified by evaluating its efficiency in decreasing mRNA levels of FAPP2 (for instance by means of Real Time PCR).

### DETAILED DESCRIPTION

The present invention provides, among other things, methods and composition of reducing globotrioaosylceramide (Gb3) accumulation and treating diseases, disorders or conditions associated with Gb3 accumulation based on inhibitors of phosphatidylinositol-4-phosphate adaptor-2 (FAPP2), including siRNAs and small molecule compounds based inhibitors. The present invention is particularly useful in treating Fabry disease and other sphingolipidoses relating to sphingolipid metabolism, such as Gaucher's disease.

Various aspects of the invention are described in detail in the following sections.

The use of sections is not meant to limit the invention. Each section can apply to any aspect of the invention. In this application, the use of “or” means “and/or” unless stated otherwise.

### Fabry Disease and Other Sphingolipidoses

Fabry disease is a glycosphingolipid (GSL) lysosomal storage disorder resulting from an X-linked inherited deficiency of lysosomal α-galactosidase A (α-GAL), an enzyme responsible for the hydrolysis of terminal α-galactosyl residues from glycosphingolipids (Brady et al. N Engl J Med. 1967; 276: 1163-7). A deficiency in α-GAL activity results in a progressive deposition of neutral glycosphingolipids, predominantly globotriaosylceramide (also known as ceramide trihexoside, CD77, Gb3), in the cells of Fabry patients.

The frequency of the classical form of disease is estimated to be about 1:40,000 to 1:60,000 in males, and is reported throughout the world within different ethnic groups. Classically affected males have little or no detectable α-GAL levels and are the most severely affected. Certain of the mutations cause changes in the amino acid sequence of α-GAL that may result in the production of α-GAL with reduced stability that does not fold into its correct three-dimensional shape. Although α-GAL produced in patient cells often retains the potential for some level of biological activity, the cell's quality control mechanisms recognize and retain misfolded α-GAL in the endoplasmic reticulum, or ER, until it is ultimately moved to another part of the cell for degradation and elimination. Consequently, little or no α-GAL moves to the lysosome, where it normally hydrolyzes Gb3. This leads to accumulation of Gb3 in cells, particularly in the vascular endothelium, which is believed to be the cause of the symptoms of Fabry disease. In addition, accumulation of the misfolded α-GAL enzyme in the ER may lead to stress on cells and inflammatory-like responses, which may contribute to cellular dysfunction and disease.

Symptoms of Fabry disease can be severe and debilitating, including kidney failure and increased risk of heart attack and stroke. While symptoms can vary from patient to patient, common symptoms of Fabry disease include: intermittent acroparesthesia (“Fabry crisis” which often manifests as a burning in the hands and feet), with episodes of acute pain lasting from hours to days; angiokeratomas (small, raised reddish-purple blemishes on the skin); cornea verticillata; hypohydrosis or anhydrosis (decreased ability to sweat); heat, cold and exercise intolerance; mild proteinuria; and gastrointestinal disorders (see Eng et al., Fabry disease: Baseline medical characteristics of a cohort of 1765 males and females in the Fabry registry, 2007, J. Inherit. Metab. Dis., 30: 184-192). Common cardiac complications of Fabry disease include left ventricular hypertrophy, heart valve disease, coronary artery disease, conduction abnormalities, heart failure, arrhythmias and acute myocardial infarction (see Pieroni et al., Fabry's disease cardiomyopathy: Echocardiographic detection of endomyocardial glycosphingolipid compartmentalization, 2006, J. Am. Coll. Cardiol., 47: 1663-1671). Common cerebrovascular symptoms of Fabry disease include white matter lesions, paresthesias, vertigo, early stroke and transient ischemic attacks (see Politei and Capizzano, Magnetic resonance image findings in 5 young patients with Fabry disease, 2006, Neurologist, 12: 103-105). In addition, damage to the glomerular podocytes can lead to proteinuria and/or hematuria. In some patients, manifestations of Fabry disease follow an oligosymptomatic course, for example, where symptoms are confined to a single system such as the renal system or cardiovascular system.

Unlike many lysosomal storage disorders, Fabry disease often afflicts young adults. For example, in the classic form of the disease, clinical manifestations may begin at age 5. Damage to organs and systems is typically progressive, and by the third to fifth decade of life most Fabry patients have developed severe kidney and heart disease. Progressive renal dysfunction eventually requires dialysis and renal transplantation, and is the main cause of death in Fabry sufferers. The life span of affected males is reduced, and death usually occurs in the fourth or fifth decade as a result of vascular disease of the heart, brain, and/or kidneys.

Other Diseases, Conditions, or Disorders

Other diseases, disorders or conditions having FAPP2 related etiology or component include, for example and without limitation, diseases, disorders or conditions including lysosome impairment as a characteristic thereof, such as a primary or secondary characteristic. It is contemplated that some embodiments may be used to treat any disease, disorder or condition having a FAPP2 component, such as, for example, Gb3 accumulation. In certain embodiments, diseases, disorders, or conditions having FAPP2 related etiology may be certain lysosomal storage disorders, in particular, sphingolipidoses. In some embodiments, a form of sphingolipidosis is Gaucher's disease.

Gaucher's disease is a genetic disease wherein lipids accumulate in cells and certain organs of sufferers. Gaucher's disease is thought to be caused by the dysfunctional metabolism of sphingolipids, specifically glucocerebrosidase. Glucocerebrosidase normally acts on the fatty acid glucosylceramide and defects in glucocerebrosidase function result in glucosylceramide accumulation, particularly in white bloods cells such as macrophages. As a result, glucosylceramide often collects in the spleen, liver, kidneys, longs, brain and bone marrow of Gaucher's sufferers. Symptoms of Gaucher's disease vary but may include enlarged spleen (splenomegaly) and/or liver (hepatomegaly), liver dysfunction such as cirrhosis, hypersplenism, pancytopenia, bone lesions, osteoporosis, swelling of lymph nodes, anemia, low blood platelet count, sclera, neuropathy, and lowered resistance to infection.

### Inhibitors of FAPP2

As discussed in the Examples below, the present inventors have discovered that phosphatidylinositol-4-phosphate adaptor-2 (FAPP2) specifically controls the synthesis of globotrioaosylceramide (Gb3). Therefore FAPP2 represents a novel target for diseases, disorders or conditions associated with Gb3 accumulation Inhibitors of FAPP2 can be used to effectively reduce Gb3 accumulation and provide novel therapy for related diseases, disorders and conditions including Fabry disease.

Inhibitors of FAPP2 suitable for the invention can be chemical compounds (e.g., small molecules), proteins or peptides, antibodies, co-crystals, nano-crystals, nucleic acids (e.g., DNAs, RNAs, DNA/RNA hybrids, siRNAs, shRNAs, miRNAs, ribozymes, aptamers, etc.), carbohydrates (e.g. mono-, di-, or poly-saccharides), lipids (e.g., phospholipids, triglycerides, steroids, etc.), natural products, any combination thereof.

Small Molecules

In some embodiments, suitable inhibitors of FAPP2 are small molecule compounds. In particular, those small molecule compounds that have similar structure as that of the glucosyl moiety of GlcCer may be particularly effective in inhibiting FAPP2. Thus, in some embodiments, a suitable FAPP2 inhibitor is an aryl glycoside. In some embodiments, a suitable FAPP2 inhibitor is an aryl C-glucoside. In some embodiments, a suitable FAPP2 inhibitor is an aryl O-glucoside.

In some embodiments, suitable inhibitors of FAPP2 include those of formula I:

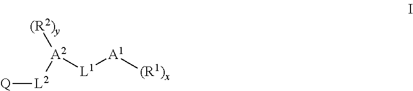

or a pharmaceutically acceptable salt thereof, wherein:
Q is a monosaccharide or modified monosaccharide;
A¹ is phenyl or a 5-6 membered heteroaryl ring having 1-3 heteroatoms independently selected from nitrogen, oxygen and sulfur;
A² is phenyl or a 5-6 membered heteroaryl ring having 1-3 heteroatoms independently selected from nitrogen, oxygen and sulfur;
L¹ is a covalent bond, or a C₁₋₄ bivalent straight or branched hydrocarbon chain, wherein one or two methylene units of the chain are optionally and independently replaced by —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
L² is a covalent bond or —O—;
each R¹ is independently halogen, —CN, —R; —OR; —SR; —N(R)₂; —N(R)C(O)R; —C(O)N(R)₂; —N(R)C(O)N(R)₂; —N(R)C(O)OR; —OC(O)N(R)₂; —N(R)S(O)₂R; —S(O)₂N(R)₂; —OC(O)OR; —C(O)R; —OC(O)R; —C(O)OR; —S(O)R; —S(O)₂R; or Cy;
each R² is independently halogen, —CN, —R, —OR, —SR, —N(R)₂, —N(R)C(O)R, —C(O)N(R)₂, —N(R)C(O)N(R)₂, —N(R)C(O)OR, —OC(O)N(R)₂, —N(R)SO₂R, —S(O)₂N(R)₂, —C(O)R, —C(O)OR, —OC(O)R, —S(O)R, or —S(O)₂R;
Cy is a ring, substituted with p instances of R³; wherein said ring is selected from the group consisting of a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R is independently hydrogen, deuterium, or an optionally substituted group selected from C₁₋₆ aliphatic; a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, —B(OR)₂, or an optionally substituted ring selected from phenyl and 5-6 membered heteroaryl having 1-4 heteroatoms independently selected from nitrogen, oxygen, and sulfur;
   p is 1-5;
   x is 0-5; and
   y is 0-4.

As defined generally herein, Q is a monosaccharide or modified monosaccharide. In some embodiments, Q is a hexose. In some embodiments, Q is a modified monosaccharide. In some embodiments, Q is 2-deoxyglucosyl. As used herein, the term “modified monosaccharide” refers to a monosaccharide wherein any one or more hydroxyl groups of an unmodified monosaccharide are replaced by a moiety independently selected from the group consisting of halogen, —CN, —R, —OR, —SR, —N(R)₂, —N(R)C(O)R, —C(O)N(R)₂, —N(R)C(O)N(R)₂, —N(R)C(O)OR, —OC(O)N(R)₂, —N(R)SO₂R, —S(O)₂N(R)₂, —C(O)R, —C(O)OR, —OC(O)R, —S(O)R, and —S(O)₂R.

As defined generally above, A¹ is phenyl or a 5-6 membered heteroaryl ring having 1-3 heteroatoms independently selected from nitrogen, oxygen and sulfur. In some embodiments, A¹ is phenyl. In some embodiments, A¹ is thiophene.

As defined generally above, A² is phenyl or a 5-6 membered heteroaryl ring having 1-3 heteroatoms independently selected from nitrogen, oxygen and sulfur. In some embodiments, A² is phenyl.

As defined generally above, L¹ is a covalent bond, or a C₁₋₄ bivalent straight or branched hydrocarbon chain, wherein one or two methylene units of the chain are optionally and independently replaced by —N(R)—, —N(R)C(O)—, —C(O)N(R)—, —N(R)S(O)₂—, —S(O)₂N(R)—, —O—, —C(O)—, —OC(O)—, —C(O)O—, —O—, —S—, —S(O)— or —S(O)₂—. In some embodiments, L¹ is —C(O)CH₂CH₂—. In some embodiments, L1 is —CH₂—.

As defined generally above, L² is a covalent bond or —O—. In some embodiments, L² is a covalent bond. In some embodiments L² is —O—.

As defined generally above, each R¹ is independently halogen, —CN, —R; —OR; —SR; —N(R)₂; —N(R)C(O)R; —C(O)N(R)₂; —N(R)C(O)N(R)₂; —N(R)C(O)OR; —OC(O)N(R)₂; —N(R)S(O)₂R; —S(O)₂N(R)₂; —OC(O)OR; —C(O)R; —OC(O)R; —C(O)OR; —S(O)R; —S(O)₂R; or Cy. In some embodiments, an R¹ is —OH. In some embodiments, an R¹ is —OEt. In some embodiments, an R¹ is phenyl 4-fluorophenyl. In some embodiments, an R¹ is tetrahydrofuranyloxy. In some embodiments, an R¹ is 2-cyclopropyloxy-ethoxy.

As defined generally above, each R² is independently halogen, —CN, —R, —OR, —SR, —N(R)₂, —N(R)C(O)R, —C(O)N(R)₂, —N(R)C(O)N(R)₂, —N(R)C(O)OR, —OC(O)N(R)₂, —N(R)SO₂R, —S(O)₂N(R)₂, —C(O)R, —C(O)OR, —OC(O)R, —S(O)R, or —S(O)₂R. In some embodiments, an R² is —OH. In some embodiments, an R² is chloro. In some embodiments, an R² is methyl.

As defined generally above, Cy is a ring, substituted with p instances of R³; wherein said ring is selected from the group consisting of a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur. In some embodiments, Cy is phenyl substituted with p instances of R³, wherein p is 1 and R³ is fluoro.

As defined generally above, each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, —B(OR)₂, or an optionally substituted ring selected from phenyl and 5-6 membered heteroaryl having 1-4 heteroatoms independently selected from nitrogen, oxygen, and sulfur. In some embodiments, an R³ is fluoro.

As defined generally above, p is 1-5. In some embodiments, p is 1.

As defined generally above, x is 0-5. In some embodiments, x is 1.

As defined generally above, y is 0-4. In some embodiments y is 1. In some embodiments, y is 2.

In some embodiments, suitable inhibitors of FAPP2 have a structure of formula II-a or II-b:

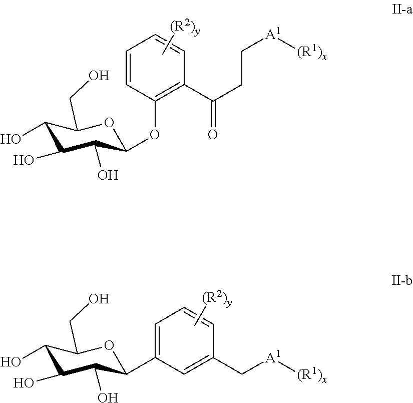

or a pharmaceutically acceptable salt thereof, wherein each of A¹, R¹, R², x, and y is as described in embodiments for formula I, supra, or described in embodiments herein, both singly and in combination.

In some embodiments, a suitable FAPP2 inhibitor is selected from the following species, or a pharmaceutically acceptable salt thereof:

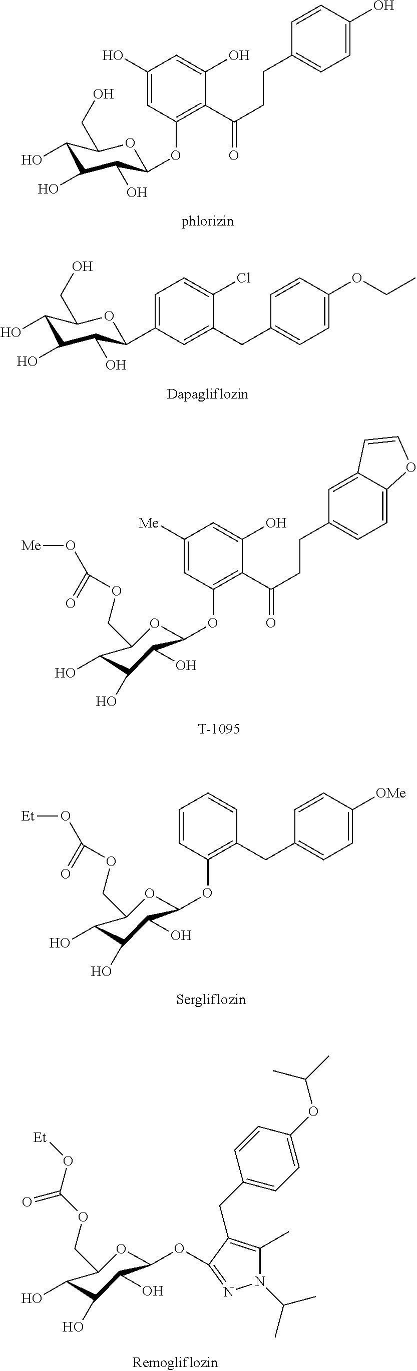

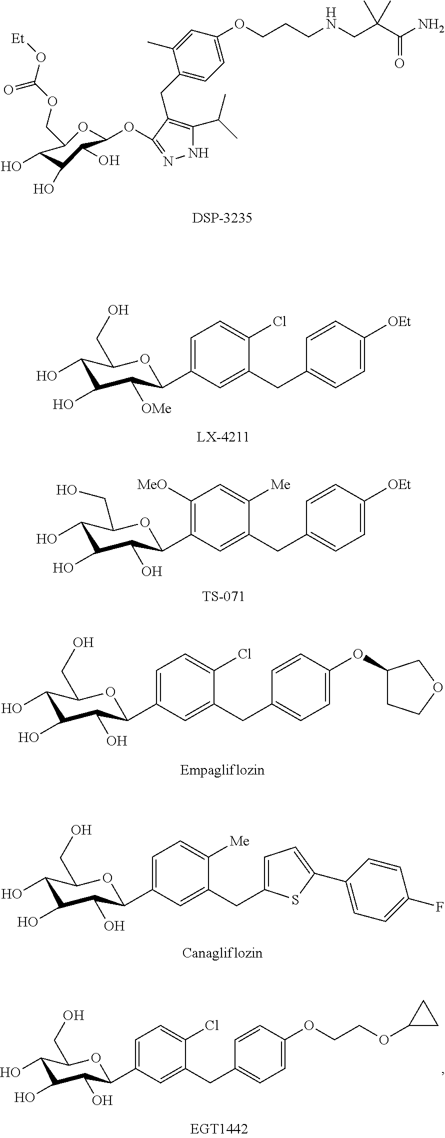

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

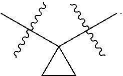 [SMILES: *C1(*)CC1]

As used herein, the term “cyclobutylenyl” refers to a bivalent cyclobutyl group of the following structure:

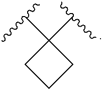 [SMILES: *C1(*)CCC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, naphthyl, anthracyl and the like, which may be optionally substituted. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R°; —(CH₂)₀₋₄OR°; —O(CH₂)₀₋₄R°, —O—(CH₂)₀₋₄C(O)OR°; —(CH₂)₀₋₄CH(OR°)₂; —(CH₂)₀₋₄SR°; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; —NO₂; —CN; —N₃; —(CH₂)₀₋₄N(R°)₂; —(CH₂)₀₋₄N(R°)C(O)R°; —N(R°)C(S)R°; —(CH₂)₀₋₄N(R°)C(O)NR°₂; —N(R°)C(S)NR°₂; —(CH₂)₀₋₄N(R°)C(O)OR°; —N(R°)N(R°)C(O)R°; —N(R°)N(R°)C(O)NR°₂; —N(R°)N(R°)C(O)OR°; —(CH)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —OC(O)(CH₂)₀₋₄SR—, SC(S)SR°; —(CH₂)₀₋₄SC(O)R°; —(CH₂)₀₋₄C(O)NR°₂; —C(S)NR°₂; —C(S)SR°; —SC(S)SR°, —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂OR°; —(CH₂)₀₋₄OS(O)₂R°; —S(O)₂NR°₂; —(CH₂)₀₋₄S(O)R°; —N(R° S(O)₂NR°₂; —N(R° S(O)₂R°; —N(OR°)R°; —C(NH)NR°₂; —P(O)₂R°; —P(O)R°₂; —OP(O)R°₂; —OP(O)(OR°)₂; SiR°₃; —(C₁₋₄ straight or branched alkylene)O—N(R°)₂; or —(C₁₋₄ straight or branched)alkylene)C(O)O—N(R°)₂, wherein each R° may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R° (or the ring formed by taking two independent occurrences of R° together with their intervening atoms), are independently halogen, —(CH₂)₀₋₂R., -(haloR.), —(CH₂)₀₋₂OH, —(CH₂)₀₋₂OR., —(CH₂)₀₋₂CH(OR.)₂; —O(haloR.), —CN, —N₃, —(CH₂)₀₋₂C(O)R., (CH₂)₀₋₂C(O)OH, —(CH₂)₀₋₂(C(O)OR., —(CH₂)₀₋₂SR., —(CH₂)₀₋₂SH, —(CH₂)₀₋₂NH₂, —(CH₂)₀₋₂NHR., —(CH₂)₀₋₂NR.₂, —NO₂, —SiR.₃, —OSiR.₃, —C(O)SR., —(C₁₋₄ straight or branched alkylene)C(O)OR., or —SSR. wherein each R. is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R° include ═O and ═S.

Suitable divalent substituents on a saturated carbon atom of an “optionally substituted” group include the following: ═O, ═S, ═NNR.₂, ═NNHC(O)R., ═NNHC(O)OR., ═NNHS(O)₂R., ═NR., ═NOR., —O(C(R.₂))₂₋₃O—, or —S(C(R.₂))₂₋₃S—, wherein each independent occurrence of R. is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR.₂)₂₋₃—, wherein each independent occurrence of R. is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R. include halogen, —R., -(haloR.), —OH, —OR., —O(haloR.), —CN, —C(O)OH, —C(O)OR., —NH₂, —NHR., —NR.₂, or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R†, —NR†₂, —C(O)R†, —C(O)OR†, —C(O)C(O)R†, —C(O)CH₂C(O)R†, —S(O)₂R†, —S(O)₂NR†₂, —C(S)NR†₂, —C(NH)NR†₂, or —N(R†)S(O)₂R†; wherein each R† is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R†, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R† are independently halogen, —R., -(haloR.), —OH, —OR., —O(haloR.), —CN, —C(O)OH, —C(O)OR., —NH₂, —NHR., —NR.₂, or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977,66,1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Assays for Identifying Additional Small Molecule Compound Inhibitors

Candidate inhibitors can also be designed using computer-based rational drug design methods, such as homology modeling based on GLTP-domain of FAPP2, the GLTP itself and the interaction of GlcCer with FAPP2. Typically, a plurality of candidate inhibitors (e.g., libraries of small molecule compounds) are tested in in vitro screening assays for potential inhibitors.

In some embodiments, public libraries containing drugs (including FDA approved drugs) can be screened to identify existing compounds whose FAPP2 modulatory activities are previously unknown. In some embodiments, modified libraries containing derivatives or analogues of existing compounds can be synthesized using methods well known in the art and screened to identify novel or improved FAPP2 inhibitors. Suitable small molecule compound libraries can be obtained from commercial vendors such as ChemBridge Libraries (www.chembridge.com), BIOMOL International, ASINEX, ChemDiv, ChemDB, ICCB-Longwood. In addition, compound libraries synthesized de novo can be screened to identify novel compounds that have specific FAPP2 inhibitory activity. In some embodiments, compounds can be synthesized using rational drug design techniques based on, for example, crystal structure of FAPP2.

Suitable in vitro assays may be based on the ability of FAPP2 to transfer GlcCer from donor to acceptor. For example, a FRET-based GlcCer transfer assay may be designed based on acceptor vesicles, donor vesicles containing fluorescent labled GlcCer, a quencher, and recombinant FAPP2 protein Inhibitors may be identified based on the recovery of emission intensity that occurs during FAPP2-mediated transfer of GlcCer from quenched donor vesicles to unquenched acceptor vesicles. An exemplary in vitro assay is described in detail in the examples section. Additional assays are known in the art and can be adapted according to the present invention.

Interfering Oligonucleotides

In some embodiments, the present invention provides interfering oligonucleotides useful for inhibiting FAPP2. In some embodiments, interfering oligonucleotides are single stranded. In some embodiments, interfering oligonucleotides are double stranded. In some embodiments, interfering oligonucleotides are antisense oligonucleotides. In some embodiments, interfering oligonucleotides are double stranded RNA molecules, for example siRNAs or shRNAs. An interfering oligonucleotide suitable for the present invention includes any oligonucleotide that is capable of inhibiting, decreasing, reducing, or down-regulating FAPP2 expression or activity.

Typically, an interfering oligonucleotide capable of down-regulating or decreasing the expression of the human FAPP2 gene may be designed based on the sequence of the human FAPP2 gene or a messenger RNA (mRNA) of FAPP2. The FAPP2 genomic sequence (NC_000007.13, gi1224589819:30054478-30171458 Homo sapiens chromosome 7, GRCh37.p10 Primary Assembly) is available at the National Center for Biotechnology Information database (NCBI) at: www.ncbi.nlm.nih.gov/nuccore/NC_000007.13?report=fasta&from=30054478&to=30171458 as of Jul. 25, 2013, and is hereby incorporated by reference in its entirety. Any isoform of FAPP2 mRNA is contemplated as within the scope of the present invention and Isoform 1, 2, and 3 are shown in Table 1 below.

TABLE 1
Human FAPP2 Isoform 1, 2 and 3 mRNA and amino acid sequences

| | | | | |
|---|---|---|---|---|
| Name | | | | Isoform1 |
| FAPP2 | | | | (multiple |
| mRNA, | Gene | | | putative |
| transcript | PLEKHA8 | Accession No. | | isoforms |
| variant 1. | (Gene ID: 84725) | NM_001197026 | SEQ ID NO: | exist) |

gi|308153326|ref|NM_001197026.1| Homo sapiens pleckstrin homology
domain containing, family A (phosphoinositide binding specific) member
8 (PLEKHA8), transcript variant 1, mRNA
CCCTGGGCATGCGCGAGCGCGTCCCGGGCCCGGCGAGTCGAGGGTTCAGGTGGTGCGCCGTGGCGCCGCC
TGCGACCGGCAGCTCGTTCGCCGCACTTTGGAGGCTTCGGCTGCCCCTCCGACCCACGTAGGGCCCGGAC
CCGGGCCTCCTTGTGAACAGCGTGCCGGCTTCGCCCCACGGGTTCACCGGCTGGCTGGGCTTCAAGCGCC
GAGGCCGCCGCAGTGACCCCGCCCCCGGGCCGAGGATGTGAGGCGGGCCGGGCGTCCCCACACCGGGCCC
GGGCGCCGGGAGTGGGCGTCTGGGCAGCGCCAGGCGATGGCCCTGCTGCTGGTGCTCCTCGCCTCTTGGG
GCCTGGGGCAGTGAGGGGGCCGGCGGGCGTGGGCCGAGTGGCCGCGGGCGCCATGGAGGGGGTGCTGTAC
AAGTGGACCAACTATCTGAGCGGTTGGCAGCCTCGATGGTTCCTTCTCTGTGGGGGAATATTGTCCTATT
ATGATTCTCCTGAAGATGCCTGGAAAGGTTGCAAAGGGAGCATACAAATGGCAGTCTGTGAAATTCAAGT
TCATTCTGTAGATAATACACGCATGGACCTGATAATCCCTGGGGAACAGTATTTCTACCTGAAGGCCAGA
AGTGTGGCTGAAAGACAGCGGTGGCTGGTGGCCCTGGGATCAGCCAAGGCTTGCCTGACTGACAGTAGGA
CCCAGAAGGAGAAAGAGTTTGCTGAAAACACTGAAAACTTGAAAACCAAAATGTCAGAACTAAGACTCTA
CTGTGACCTCCTTGTTCAGCAAGTAGATAAAACAAAAGAAGTGACCACAACTGGTGTGTCCAATTCTGAG
GAGGGAATTGATGTGGGAACTTTGCTGAAATCAACCTGTAATACTTTTCTGAAGACCTTGGAAGAATGCA
TGCAGATCGCAAATGCAGCCTTCACCTCTGAGCTGCTCTACCGCACTCCACCAGGATCACCTCAGCTGGC
CATGCTCAAGTCCAGCAAGATGAAACATCCTATTATACCAATTCATAATTCATTGGAAAGGCAAATGGAG
TTGAGCACTTGTGAAAATGGATCTTTAAATATGGAAATAAATGGTGAGGAAGAAATCCTAATGAAAAATA
AGAATTCCTTATATTTGAAATCTGCAGAGATAGACTGCAGCATATCAAGTGAGGAAAATACAGATGATAA
TATAACAGTCCAAGGTGAAATAAGGAAGGAAGATGGAATGGAAAACCTGAAAAATCATGACAATAACTTG
ACTCAGTCTGGATCAGACTCAAGTTGCTCTCCGGAATGCCTCTGGGAGGAAGGCAAAGAAGTTATCCCAA
CTTTCTTTAGTACCATGAACACAAGCTTTAGTGACATTGAACTTCTGGAAGACAGTGGCATTCCCACAGA
AGCATTCTTGGCATCATGTTATGCTGTGGTTCCAGTATTAGACAAACTTGGCCCTACAGTGTTTGCTCCT
GTTAAGATGGATCTTGTTGGAAATATTAAGAAAGTAAATCAGAAGTATATAACCAACAAAGAAGAGTTTA
CCACTCTCCAGAAGATAGTGCTGCACGAAGTGGAGGCGGATGTAGCCCAGGTTAGGAACTCAGCGACTGA
AGCCCTCTTGTGGCTGAAGAGAGGTCTCAAATTTTTGAAGGGATTTTTGACAGAAGTGAAAAATGGGGAG
AAGGATATCCAGACAGCCCTAAATAATGCATATGGTAAAACATTGCGGCAACACCATGGCTGGGTAGTTC
GAGGGGTTTTTGCGTTAGCTTTAAGGGCAGCTCCATCCTATGAAGATTTTGTGGCCGCGTTAACCGTAAA
GGAAGGTGACCACCAGAAAGAAGCTTTCAGTATTGGGATGCAGAGGGACCTCAGCCTTTACCTCCCTGCC
ATGGAGAAGCAGCTGGCCATACTGGACACTTTATATGAGGTCCACGGGCTGGAATCTGATGAGGTGGTAT
GATGGCTGCTGGGCAGCACCTCCTAACTTCAGGGAATAAGTGCTAAAGTGTTTTGTTGCCCTACTTAATT
TCCAGCAACAGCCTCAACCCTCTCCAACCCCTTCACCTGGGGGGATGGACAGGAGGTGGCAAAACCCAGT
GCTTTTATAATTTTTAAAATGCATATGTGTTTTGTTTAAAGATCAAGGTGCTATATATTTCAGTTCAGCA
GGCCTACTGGAAACCAAATGATAAGCTGCTGTAGACTTGAACAGCAAGTTATAAGAGCAGATTTAACAAA
CAAATTTGCTGTTATTGTGTATTGTATTGTTTTTATATTTTAGTCTAATGGGCCACCCAAACCCAAGCTG
AAAATCAGCAAATTCCATATTAAGTACCATAATTCATAGCCAGTGTTTCAGCCAACTTAGACTAGACATT
TGGAGGTAGTATAAGCTGCTTTGTTGAAGCTGTGTAGAGTTTGCTGTTCCTAGATGTTCTTCAGTGGACC
CTCTTCACTGCAACTCTGTCAGTGATAAGGGCCTGTGTAGTAAAGATGTTCAGGGCATTCACATGACCAT
GCAATTGTGGGAGGCGAAGAAGACGTGGACAGGAGTCCCATCCTTGCTGACAGGCATGAAACCGTTGCTC
TGAGAAGATTAATGGTGTGCCCTAGCCCCAAGTTGGAGGGGAGAATATGAGAGAGGTGGGACAGGTCATT
TGAGATGACACCTCCCAACTGCCTACCATTTACCAGCATGTTCCCCATGCATTATCTCAATTGGACATCA
CAAGTAATGATACCCAGAGGGATTATTACTCCACTTCAAAAGCAAGGTTTAGAAGTTGAGGGATCTGTTC
ACAGTCACATAGTTTTTAAGCAGAGGAGCCAGATAATTTCCAAGTGTGACCTGGACTGCCTCTGCATCAA
AGTCATATGGAGTGCTTGTTCAAACAGCAGATTCCCAGGCCTTATTTTGGCCTAAAGAACCAGAGTCTAG
GTGGTGGGACATAGGAATCTGCATTTCAGTAAACTTTACACGTGATTCTTCTGCACACAGTATTGAAGAG
CAACTAGATTAAATTCTAGTTTACAAAATTACCAGTTTTCTTCAAGAACTAAATGATATGTCCTTTTTTT
TTTTTTCAAAGAGGATAAGGCTGCTATTTAAATAAAATAGCTAAATGGAGAGTGAGAAGTGGAGCAGGTT
CATTCAGCAGCATTCTTAATTGAGCCAGCATTGACACCCAGCCAGCAGGCCTTTGCATTGCATTCGGGGA
CCATGACTCTGAATCTGCTTACCAATCAATCTCGGTTTAATCACCAAAAGTGCAGAGCAGGCAAAATGCA
GCTGTTTATCAATCTCAAAAGCTTTGGGACAGTGTCATAGTTGAAAGATGAGACTTAAGAAAACAGTTTC
TTAAACTTCTTAAAACTTAAGAAACATTGTTTCATAAAACAATATTGAGTGGGCATTCTTCTGCACAGTG
TGATGCTCCAACCCTGGCCCTAGTCTCAGTAGACCATGCTGCCTCGAGTGTGCATCGGAGAGAAGCCATG
GGTACCTTCCCCATTAGAGGCTACTTCCTTCTAGTAACAGGAAGGGAAGTTCCAGCATGAGGTAGTTATC
CAGGGTAGAAGGTCCTTTGAGGGGCTTGGTTGAATTGAGAGCATCATCTCTAGATGATGCTGTTCCTGCT
GCAGATCTCTAGGATGGAGAGAATTCTCTCTTTAGTCAGAGAAGTTTATGTAGGGAGGGGTATTGGTTTT
GCCTTTGTGTGTCTTTAAACAAATGAACATTTATTTAGCTCAGATTAATTAGGTATTTTGCCCACATAAA
GACTTCTGGAAAATACTTAAACTTGAAAAATCAACATCACATGTTTTAAAGCTAGGGAGAAAGAAGGGGG
GTATTAAAATGATGTTGATTATTTTGTATTTTGCCAAGGTGTGTGTGTGTTATTTCCCTCCCACTCTCAT
GAGCAGTGAGTATAGATCTCCTTCTCTGATTAGTATGAATATGATGGCAGGACTCGGGGATAGTCCCTGC
CCTTGACATAGCCCCCTAAAACGGAAAAAGAAAAAGCCAGTTTTTGCTTGTACTTTGAATGAAGATGTTT
TAGGCATTGTACCATTTAGCGGGGATGATACCAGGTGGTTGTTAGAATTGTGCAGTGTGATCATTCTAAA
CAGCTGCTGGTGCTCCCTGTCACCTCAGGTGAACTCTGTGGTCTCTTGGAGAGGTAGCACTCTGAAAATA
CCTCAGGTTTGCCACCGCAACTCTGAATACACACAAAAGGAAAGCTGCTCAGCATGGCCATTTTGCATTT
GTATAGGTAGTGACTAGATGTACACAACTTAATTTGCTGGGAATGAGGGGCTTAATATTATCTGAGATCA
TTGAGAACCCAGATCAGACAGAATAGCTTGAATAAGTTACATTTTCCAATTACCCTTTTTCCACATCTGT
AGAAAGAGGGTAATATTTTTTAATAGGTATTTTCCCCACTGGAGCATATTACGTTTGCCTAAGATGTATA
AAAGTTTGTTTAAGATGTGTAAAAGTTTGCTTAAGGAACTGAGGATCCTTAAATAAAAATATTAGAAATT
AGAAATTGAACCTAATACTAAACAGTAAATTCAGCTTAACCTGAACCTTGGCATAGTCAGAGCTTCCTCC
TACATCTAAAGTATTTGCTCTCTGTTTTAGTTAAAGTCATAATTTGCGCTGATGTGTAATCACTTTCCAA
GAAGAGGGCAATGAGAAAAGATATTTAAAGCTTTCTCTCCATAGCCCTCCAAGACTTCTGGGACAACTAA
ATTTACTTTCACCATTACTGTGAGAGGAGGTGAGAAAACTCTAGTATTTTGTTGGCAGAGTAATCACTTT
GTTCTCATCGCTCAAAGCATTTTTAGGATTATTTTTCTAGCGTAACCTTTAGAGAGAACTGGAAGAAAAA
GGAAATTGGTCTACTAGGTATTGTAGACACAAATAAGTAACATTAGGCTAACCCCTTATGAGACATTTCC
ACACAATTTCATCGTGCCTGTACTTTTCTCTATGGTAAAAGCCAGTGTTTACACTTTGTAGGGATCAGGG
TGTATTTGTTGAATTAAACAAAATATTTTCAATGATGGCAAGTCTCTTGACTTTTGAAAGCAAGTCAGAT
TCCTTATAGCTAATGCTGGTGAAAAATGTTAAATTGGAGAGATCCCTTTTGGGAGTGAAACCAAATTGTA
ACTATGAGGAGAAGATGGTCTTCTCATTGGCTCTTGATGTAGCTCTGAAGGGAGTTCCAGAAGAGGAGCT
CTCACAGAATGTTGAGCCTGTGGGCCCAAGACATTGACTTCGAAGGGTAGTTCTCATTAGGATGTATAAG
TAGTGGCTTGAGGCACCTTCTTATCATTTTTTGCATGTTATTCTGATTATTAAACTTCCCCCAATGTCAT
ATTCCATGATGAGGGATTTCTGAACTCCATAGTCCAGCGTTGTTGCTTTTCTCTCTTCTTTGCTACTGAA
AATTGCCAGCAGTACCGCCATCAGCACACCAAATCTACCCCCACTTATGTTTGTTCTGCCCCATTTCCCA
GGAGCAGCTTCTAGCACATATGTAGAGTATCTGGCACCACCTTAGCCCAGGGCTGCGTGCCTGATCAGTG
GGGATTCTGTTCCCCCACCCCCCAGACTGCAAGAGCTTCTTAAGAAGGAGCCCATATTCCCATTTGTAGC
TGGAAAGCGGGTGAATGACATGACATGGGGCACCTAGGAAAGATGATTATTAGAGGAGTGCAGCGGAAAA
AAATTTGCACTCTTCTCCTTTTGGTTATTACTTTCCAAATATATTAACAAAAAGTTGATGCTTTTAACTT
TATATTTTCAGAAAAGTGTTTTTAATTAAAAATATGTGATAGGGACCAAATAAGTAAAGTACATTTTTCT
CCACTAAATTTGAAGTGAGGGAAAGAGGAGCCAAAGTAAGAGATTTTTTTTTAAGGAAACTTAATCTGAT
TGTGAAAATCATACATATGGAGAAACATCAGATCAGGCAATAGAGTCAGAGGGTCATGAGCAATAGACGA
TGATGCGAGGCATTTGGGGAGCTTCCTGGAGGAAAATTAAGTTTTTTTCCTAGCAAACTACCATGTCCTA
CAAGAACTTGGTTATATAATGGTGCGTCTCTGAATCACTGATTAAAACCAGTTGCTTCTGATTTTAGTCA
CAGGTTTTACAAGTATTCAGCTCTCCCTCATGTTTCATTTCTTTTTTTAAGATAATCTATCAACCTTTTT
TAAATTTTAAAATTTTTAGATGTAGAGTTTATAAGTAAAATATATTTTTAGCCATTGTTCTGTTAGCTGA
GCTGATGTGTTTGGTCTTAGAGGGCCTGACTTCAGATACTCTTTGTGATCTTGTAAGGGCTCTACACAAA
CTTCATTATGTATGGTAAATTTGTATTCTTATGGATTGTATATAGAATGCTTTCGTTAGAAGTACATTCT
ACTTCTGTATGTCCCTTTGTAATCCGCAGTTGCTTACTCAGGGGTTTCATAGTCATTTCATAAAAAATAA
TTCACTAGCTGTCTAATGGTATTTTAAGACTGTTTATCTGTATCACAACGTCATTAGGAGTTCTTTCAAC
AATTCCATAAATATACTGTTTACTAGACCTTCCCTGTAAATGTTCCCAATTCCCATCCTGTCTCAGACAG
TCAATAGTCCTGTGTACAGTGACTATTTGCATGATTTCTCATTGCACTGCTGCATTCAGGCACTCCAGGG
CATGATTAAACAGTCATTAACAGTGCCTCTCTGGTACAGTTGATGCATGCATTGATCTTTCTTCTCTGCT
GTTTTTATATAGCCTTTAATTAAAAGGAAAAAAATACCACTACTCTGCAATGCAAAAGTCTTCAAAATTC
TTTGTTTCCTGTATTAATCACTTCTGTTCCAGAGTGAACAAATGTTTTCAGCTAAGCTATGTGAGAATGT
AAGAATAATATCCTGCTTGTTCTAAATAGTTCATATATTTAAAGTGTGGTCAGTATTTCCTCCCTGTACC
TTACAAACAGAAACCACCCTGGGATGGTTGATACCCCTTACAAAGTCGATCTTACCCACACAGACTCCTG
TGTATGCGTGTCTGTTTATAGGTGTATATGGAGTCAGTGTTGATAGGAAGGATGCTCTAGAAGTACTTCT
GTTGTTTCCTAGAAGGCTATGAGCCAGTTCCATGGCATGTTTAATGTATAATTCCCATGTATCATGAGAA
TTTCACTAGAATGTCATTAAACAGCCCAACTACCTCATGTGAAATTGGCTGTGGACAATCTGTGTCAGAT
GAGAAATGTGTTCAGATAAATTTAATCTGGTTAATAGACTTAACAAATTAATGTCTACATAAAGAAGAAA
CATGATAGACCAGATGCCAAAGGCTAAAATGTACATAGATTTCCTTGGATTAATTTTTAAGTCACTGTTT
AATTCCATGCCTAGTATTCTTATGAATGTTTGTGGTTTCATAGATTTATGCACTTTGAATATCTGTCACG
TGCAGTGTTAATGTTACCTGTTCTTGTCTCTCAGCATTTTGAATGAGCATCATAATCAGAGTAGAAGGCA
AGTTAAACTATAAAAGTGTCAAGTGGCTTGTTAACTTCTTAATTTAATGGACCTTTACTTAGAATATAAT
ATGTTGGAGCCTCTTGGGACCAACCGATGAGCGACAGTTTCATGTTTAGATTTGTATTGTTTCTCTGTCC
AAGTCCTTATTCTCTATCTTGTGGGGAGGGGTGACAGGGGAGGGTTTTACTTTTTTTGCAAAAATGTTTG
AAAATATCTGTCAGATTTTATATTCGTTAGTTATAATAAACTTATTTTTAAAGTATTAAGTTCTTAAAAA
AAAAAAAAAAA
(Sequence ID No: 1)

| | | | | |
|---|---|---|---|---|
| | | | | Notes |
| Name | | | | Isoforna1 |
| FAPP2 | | | | (multiple |
| Protein, | Gene | | | putative |
| transcript | PLEKHA8 | Accession No. | | isoforms |
| variant 1. | (Gene ID: 84725) | NP_001183955 | SEQ ID NO.: | exist) |

gi|308153327|ref|NP_001183955.1| pleckstrin homology domain-containing
family A member 8 isoform 1 [Homo sapiens]
MEGVLYKWTNYLSGWQPRWFLLCGGILSYYDSPEDAWKGCKGSIQMAVCEIQVHSVDNTRMDLIIPGEQY
FYLKARSVAERQRWLVALGSAKACLTDSRTQKEKEFAENTENLKTKMSELRLYCDLLVQQVDKTKEVTTT
GVSNSEEGIDVGTLLKSTCNTFLKTLEECMQIANAAFTSELLYRTPPGSPQLAMLKSSKMKHPIIPIHNS
LERQMELSTCENGSLNMEINGEEEILMKNKNSLYLKSAEIDCSISSEENTDDNITVQGEIRKEDGMENLK
NHDNNLTQSGSDSSCSPECLWEEGKEVIPTFFSTMNTSFSDIELLEDSGIPTEAFLASCYAVVPVLDKLG
PTVFAPVKMDLVGNIKKVNQKYITNKEEFTTLQKIVLHEVEADVAQVRNSATEALLWLKRGLKFLKGFLT
EVKNGEKDIQTALNNAYGKTLRQHHGWVVRGVFALALRAAPSYEDFVAALTVKEGDHQKEAFSIGMQRDL
SLYLPAMEKQLAILDTLYEVHGLESDEVV
(Sequence ID No: 2)

| | | | | |
|---|---|---|---|---|
| | | | | Notes |
| Name | | | | Isoform2 |
| FAPP2 | | | | (multiple |
| mRNA, | Gene | | | putative |
| transcript | PLEKHA8 | Accession No. | | isoforms |
| variant 2. | (Gene ID: 84725) | NM_001197027 | SEQ ID NO.: | exist) |

>gi|308153324|ref|NM_001197027.1| Homo sapiens pleckstrin homology
domain containing, family A (phosphoinositide binding specific) member
8 (PLEKHA8), transcript variant 2, mRNA
CCCTGGGCATGCGCGAGCGCGTCCCGGGCCCGGCGAGTCGAGGGTTCAGGTGGTGCGCCGTGGCGCCGCCT
GCGACCGGCAGCTCGTTCGCCGCACTTTGGAGGCTTCGGCTGCCCCTCCGACCCACGTAGGGCCCGGACCC
GGGCCTCCTTGTGAACAGCGTGCCGGCTTCGCCCCACGGGTTCACCGGCTGGCTGGGCTTCAAGCGCCGAG
GCCGCCGCAGTGACCCCGCCCCCGGGCCGAGGATGTGAGGCGGGCCGGGCGTCCCCACACCGGGCCCGGGC
GCCGGGAGTGGGCGTCTGGGCAGCGCCAGGCGATGGCCCTGCTGCTGGTGCTCCTCGCCTCTTGGGGCCTG
GGGCAGTGAGGGGGCCGGCGGGCGTGGGCCGAGTGGCCGCGGGCGCCATGGAGGGGGTGCTGTACAAGTGG
ACCAACTATCTGAGCGGTTGGCAGCCTCGATGGTTCCTTCTCTGTGGGGGAATATTGTCCTATTATGATTC
TCCTGAAGATGCCTGGAAAGGTTGCAAAGGGAGCATACAAATGGCAGTCTGTGAAATTCAAGTTCATTCTG
TAGATAATACACGCATGGACCTGATAATCCCTGGGGAACAGTATTTCTACCTGAAGGCCAGAAGTGTGGCT
GAAAGACAGCGGTGGCTGGTGGCCCTGGGATCAGCCAAGGCTTGCCTGACTGACAGTAGGACCCAGAAGGA
GAAAGAGTTTGCTGAAAACACTGAAAACTTGAAAACCAAAATGTCAGAACTAAGACTCTACTGTGACCTCC
TTGTTCAGCAAGTAGATAAAACAAAAGAAGTGACCACAACTGGTGTGTCCAATTCTGAGGAGGGAATTGAT
GTGGGAACTTTGCTGAAATCAACCTGTAATACTTTTCTGAAGACCTTGGAAGAATGCATGCAGATCGCAAA
TGCAGCCTTCACCTCTGAGCTGCTCTACCGCACTCCACCAGGATCACCTCAGCTGGCCATGCTCAAGTCCA
GCAAGATGAAACATCCTATTATACCAATTCATAATTCATTGGAAAGGCAAATGGAGTTGAGCACTTGTGAA
AATGGATCTTTAAATATGGAAATAAATGGTGAGGAAGAAATCCTAATGAAAAATAAGAATTCCTTATATTT
GAAATCTGCAGAGATAGACTGCAGCATATCAAGTGAGGAAAATACAGATGATAATATAACAGTCCAAGGTG
AAATAAGGAAGGAAGATGGAATGGAAAACCTGAAAAATCATGACAATAACTTGACTCAGTCTGGATCAGAC
TCAAGTTGCTCTCCGGAATGCCTCTGGGAGGAAGGCAAAGAAGTTATCCCAACTTTCTTTAGTACCATGAA
CACAAGCTTTAGTGACATTGAACTTCTGGAAGACAGTGGCATTCCCACAGAAGCATTCTTGGCATCATGTT
ATGCTGTGGTTCCAGTATTAGACAAACTTGGCCCTACAGTGTTTGCTCCTGTTAAGATGGATCTTGTTGGA
AATATTAAGAAAGTAAATCAGAAGTATATAACCAACAAAGAAGAGTTTACCACTCTCCAGAAGATAGTGCT
GCACGAAGTGGAGGCGGATGTAGCCCAGGTTAGGAACTCAGCGACTGAAGCCCTCTTGTGGCTGAAGAGAG
GTCTCAAATTTTTGAAGGGATTTTTGACAGAAGTGAAAAATGGGGAGAAGGATATCCAGACAGCCCTAAAT
AATGCATATGGTAAAACATTGCGGCAACACCATGGCTGGGTAGTTCGAGGGGTTTTTGCGGGACACACAAA
TGTCTGATGGTGGCCCAGGAGGGCTGTCACTCAGAATCAGGCGGGTGCAGAGTGGCCTGCAGGACATGATG
ACCCAGCATGAGACAGGATGCATCACATCTGGGTGACGCCTCTCTCCAGGGGATCAGCCTGGACTAAATGC
CTCTCCTGGGCCAGGATGCCCAGCAAGCCCCGAGATGCCTCCTGGAGAGAAGGAAGTATGTGAGAGTATCC
TGAGTTAAGTGAGATCTAAAATGCCTGAGAAAACCCTGAAATGACTGAGTCTATCTGGAACTTATTTGATT
GTTTTTCTGCCATCAGATAGAATGAGGTCTCAAAAGAGAAATTAAATTATATTACAAGAAAATAAAGCTCA
ATTGCTTGCCAAAAAAA
(Sequence ID No: 85)

| | | | | |
|---|---|---|---|---|
| | | | | Notes |
| Name | | | | Isoform2 |
| FAPP2 | | | | (multiple |
| Protein, | Gene | | | putative |
| transcript | PLEKHA8 | Accession No. | | isoforms |
| variant 2. | (Gene ID: 84725) | NP_001183956 | SEQ ID NO.: | exist) |

>gi|308153325|ref|NP_001183956.1| pleckstrin homology domain-containing
family A member 8 isoform 2 [Homo sapiens]
MEGVLYKWTNYLSGWQPRWFLLCGGILSYYDSPEDAWKGCKGSIQMAVCEIQVHSVDNTRMDLIIPGEQYF
YLKARSVAERQRWLVALGSAKACLTDSRTQKEKEFAENTENLKTKMSELRLYCDLLVQQVDKTKEVTTTGV
SNSEEGIDVGTLLKSTCNTFLKTLEECMQIANAAFTSELLYRTPPGSPQLAMLKSSKMKHPIIPIHNSLER
QMELSTCENGSLNMEINGEEEILMKNKNSLYLKSAEIDCSISSEENTDDNITVQGEIRKEDGMENLKNHDN
NLTQSGSDSSCSPECLWEEGKEVIPTFFSTMNTSFSDIELLEDSGIPTEAFLASCYAVVPVLDKLGPTVFA
PVKMDLVGNIKKVNQKYITNKEEFTTLQKIVLHEVEADVAQVRNSATEALLWLKRGLKFLKGFLTEVKNGE
KDIQTALNNAYGKTLRQHHGWVVRGVFAGHTNV
(Sequence ID No: 86)

| | | | | |
|---|---|---|---|---|
| | | | | Notes |
| Name | | | | Isoform3 |
| FAPP2 | | | | (multiple |
| mRNA, | Gene | | | putative |
| transcript | PLEKHA8 | Accession No. | | isoforms |
| variant 3. | (Gene ID: 84725) | NM_032639 | SEQ ID NO.: | exist) |

>gi|308153328|ref|NM_032639.3| Homo sapiens pleckstrin homology domain
containing, family A (phosphoinositide binding specific) member 8
(PLEKHA8), transcript variant 3, mRNA
CCCTGGGCATGCGCGAGCGCGTCCCGGGCCCGGCGAGTCGAGGGTTCAGGTGGTGCGCCGTGGCGCCGCCT
GCGACCGGCAGCTCGTTCGCCGCACTTTGGAGGCTTCGGCTGCCCCTCCGACCCACGTAGGGCCCGGACCC
GGGCCTCCTTGTGAACAGCGTGCCGGCTTCGCCCCACGGGTTCACCGGCTGGCTGGGCTTCAAGCGCCGAG
GCCGCCGCAGTGACCCCGCCCCCGGGCCGAGGATGTGAGGCGGGCCGGGCGTCCCCACACCGGGCCCGGGC
GCCGGGAGTGGGCGTCTGGGCAGCGCCAGGCGATGGCCCTGCTGCTGGTGCTCCTCGCCTCTTGGGGCCTG
GGGCAGTGAGGGGGCCGGCGGGCGTGGGCCGAGTGGCCGCGGGCGCCATGGAGGGGGTGCTGTACAAGTGG
ACCAACTATCTGAGCGGTTGGCAGCCTCGATGGTTCCTTCTCTGTGGGGGAATATTGTCCTATTATGATTC
TCCTGAAGATGCCTGGAAAGGTTGCAAAGGGAGCATACAAATGGCAGTCTGTGAAATTCAAGTTCATTCTG
TAGATAATACACGCATGGACCTGATAATCCCTGGGGAACAGTATTTCTACCTGAAGGCCAGAAGTGTGGCT
GAAAGACAGCGGTGGCTGGTGGCCCTGGGATCAGCCAAGGCTTGCCTGACTGACAGTAGGACCCAGAAGGA
GAAAGAGTTTGCTGAAAACACTGAAAACTTGAAAACCAAAATGTCAGAACTAAGACTCTACTGTGACCTCC
TTGTTCAGCAAGTAGATAAAACAAAAGAAGTGACCACAACTGGTGTGTCCAATTCTGAGGAGGGAATTGAT
GTGGGAACTTTGCTGAAATCAACCTGTAATACTTTTCTGAAGACCTTGGAAGAATGCATGCAGATCGCAAA
TGCAGCCTTCACCTCTGAGCTGCTCTACCGCACTCCACCAGGATCACCTCAGCTGGCCATGCTCAAGTCCA
GCAAGATGAAACATCCTATTATACCAATTCATAATTCATTGGAAAGGCAAATGGAGTTGAGCACTTGTGAA
AATGGATCTTTAAATATGGAAATAAATGGTGAGGAAGAAATCCTAATGAAAAATAAGAATTCCTTATATTT
GAAATCTGCAGAGATAGACTGCAGCATATCAAGTGAGGAAAATACAGATGATAATATAACAGTCCAAGGTG
AAATAAGGAAGGAAGATGGAATGGAAAACCTGAAAAATCATGACAATAACTTGACTCAGTCTGGATCAGAC
TCAAGTTGCTCTCCGGAATGCCTCTGGGAGGAAGGCAAAGAAGTTATCCCAACTTTCTTTAGTACCATGAA
CACAAGCTTTAGTGACATTGAACTTCTGGAAGACAGTGGCATTCCCACAGAAGCATTCTTGGCATCATGTT
ATGCTGTGGTTCCAGTATTAGACAAACTTGGCCCTACAGTGTTTGCTCCTGTTAAGATGGATCTTGTTGGA
AATATTAAGAAAGTAAATCAGAAGTATATAACCAACAAAGAAGAGTTTACCACTCTCCAGAAGATAGTGCT
GCACGAAGTGGAGGCGGATGTAGCCCAGGTTAGGAACTCAGCGACTGAAGCCCTCTTGTGGCTGAAGAGAG
GTCTCAAATTTTTGAAGGGATTTTTGACAGAAGTGAAAAATGGGGAGAAGGATATCCAGACAGCCCTAAGA
AATCCAACAGAAAACACTTGACACCAAAACATACCCTGATGAAGATCCTGAACTTCAAGAATGAAGAAAGA
ATTCCTCACCATTCAGGCAGAAAAAGCAAGTCACCAAGGGACCTCAAACTTCCTTTCCACAAGATTCTGTG
ACGGGAAACAATGGGGGAGTATTTCCGAAGTTCTGAGTAGGAAAAAAGAATGACTCAAATGTATTATTGCC
AACCAAGTCGTCAAATCTAATGTCAAGTTCTCTTAAGCAGGTAAGAACTCAGAACATAATACCTGAGTGCC
TTCTTAAGGAAACCATTTGATAGGAAAGATGAACCAAATAACTCAATGATGGATGAGCTGGTAGAAAAAAA
GCTGGTGGTGAACCAAGGTCAAACTGGAAATTATAGTCACAGTATAGATATAGATTATAAATATTACAAAC
CCTAAGATAGCTAATAAATTGGGAATGGGAGAAGGGAGGATATAAGAGCACTAATGCCCTCTTATTTTCAT
AGCAGAGACTTGATACTGTCTCAACTTTTTTCAAAAACACAATTTCTTAAATTTTTTGGTAATCTTTTAAA
TAAACAGATTTCTAAAAAGAAAAAAAAAAAAAAAAAAAAAAAA
(Sequence ID No: 87)

| | | | | |
|---|---|---|---|---|
| | | | | Notes |
| Name | | | | Isoform3 |
| FAPP2 | | | | (multiple |
| Protein, | Gene | | | putative |
| transcript | PLEKHA8 | Accession No. | | isoforms |
| variant 3. | (Gene ID: 84725) | NP_116028 | SEQ ID NO.: | exist) |

>gi|14249174|ref|NP_116028.1| pleckstrin homology domain-containing
family A member 8 isoform 3 [Homo sapiens]
MEGVLYKWTNYLSGWQPRWFLLCGGILSYYDSPEDAWKGCKGSIQMAVCEIQVHSVDNTRMDLIIPGEQYF
YLKARSVAERQRWLVALGSAKACLTDSRTQKEKEFAENTENLKTKMSELRLYCDLLVQQVDKTKEVTTTGV
SNSEEGIDVGTLLKSTCNTFLKTLEECMQIANAAFTSELLYRTPPGSPQLAMLKSSKMKHPIIPIHNSLER
QMELSTCENGSLNMEINGEEEILMKNKNSLYLKSAEIDCSISSEENTDDNITVQGEIRKEDGMENLKNHDN
NLTQSGSDSSCSPECLWEEGKEVIPTFFSTMNTSFSDIELLEDSGIPTEAFLASCYAVVPVLDKLGPTVFA
PVKMDLVGNIKKVNQKYITNKEEFTTLQKIVLHEVEADVAQVRNSATEALLWLKRGLKFLKGFLTEVKNGE
KDIQTALRNPTENT
(Sequence ID No: 88)

For example, an interfering oligonucleotide capable of down-regulating or decreasing the expression of the human FAPP2 gene may have a sequence that is substantially identical to the reverse complement of a continuous sequence of the human FAPP2 gene or mRNA. In some embodiments, an interfering oligonucleotide according to the present invention has a sequence at least about 50% (e.g., at least about 55%, 60%, 65%, 70%, 75%, 80%, 85%, 90%, 95%, 96%, 97%, 98%, or 99%) to the reverse complement of a continuous sequence of the human FAPP2 gene or mRNA.

Alternatively, an interfering oligonucleotide capable of down-regulating or decreasing the expression of the human FAPP2 gene is capable of hybridizing or binding to a target region of FAPP2 mRNA.

It will be appreciated that hybridization of an interfering oligonucleotide to a target region of FAPP2 mRNA may be performed in vitro or in vivo. Hybridization may be performed under low, medium, and/or stringent hybridization conditions, as is well known in the art. In general, stringent hybridization conditions refer to standard hybridization conditions under which nucleic acid molecules, including interfering oligonucleotides, are used to identify molecules having complementary nucleic acid sequences. Stringent hybridization conditions typically permit binding between nucleic acid molecules having at least about 50%, 55%, 60%, 65%, 70%, 75%, 80%, 85%, 90%, 95%, or more nucleic acid sequence identity. Standard conditions are disclosed, for example, in Sambrook et al., 1989, Molecular Cloning: A Laboratory Manual, Cold Spring Harbor Labs Press, the contents of which is incorporated herein by reference in its entirety. Formulae to calculate the appropriate hybridization and wash conditions to achieve hybridization permitting 50%, 40%, 30%, 20%, 10%, 5% or less mismatch of nucleotides are available in the art, for example, in Meinkoth et al., 1984, Anal. Biochem. 138, 267-284; the contents of which is incorporated herein by reference in its entirety. It will be appreciated that hybrids between oligonucleotides (14-20 bp) and immobilized DNA show decreased stability and should be taken into account when defining optimal conditions for their hybridization.

Hybridization condition stringency can be affected by buffer ionic strength, base composition of the nucleotide, the length of the shortest chain in the duplex (n), and the concentration of helix destabilizing agents such as formamide. For example, hybridization stringency can be altered by adjusting the salt and/or formamide concentrations and/or by changing the temperature. The stringency can be adjusted either during the hybridization step, or in post hybridization washes. An example of stringent hybridization conditions for hybridization of complementary nucleic acids which have more than 100 complementary residues on a filter in a Southern or Northern blot is 50% formamide with 1 mg of heparin at 42° C., with the hybridization being carried out overnight. An example of stringent wash conditions is a 0.2×SSC wash at 65° C. for 15 minutes. In some embodiments, a high stringency wash is preceded by a low stringency wash to remove back-ground probe signal. An example medium stringency wash for a duplex of, e.g., more than 100 nucleotides, is 100×SSC at 45° C. for 15 minutes. An example low stringency wash for a duplex of, e.g., more than 100 nucleotides, is 4×SSC at 40° C. for 15 minutes. In general, a signal to noise ratio of 2× (or higher) than that observed for an unrelated probe in the particular hybridization assay indicates detection of a specific hybridization.

Exemplary interfering oligonucleotides suitable for the present invention are listed in Table 2:

TABLE 2
Exemplary Short Interfering Oligonucleotides
(siRNAs (oligo 1-4) and shRNAs
(oligo 5-8)) to FAPP2

| Oligo # | Sequence (5′-3′) | SEQ ID NO: |
|---|---|---|
| 1 | GAGAUAGACUGCAGCAUAU[dT][dT] | 3 |
| 2 | GAAUUGAUGUGGGAACUUU[dT] | 4 |
| 3 | GAAAUCAACCUGUAAUACU[dT][dT] | 5 |
| 4 | CCUAAGAAAUCCAACAGAA[dT][dT] | 6 |
| 5 | CTCTTGTGGCTGAAGAGAGGTCTCAAATT | 7 |
| 6 | TTGGCAGCCTCGATGGTTCCTTCTCTGTG | 8 |
| 7 | CAGTCTGGATCAGACTCAAGTTGCTCTCC | 9 |
| 8 | TCCTGTTAAGATGGATCTTGTTGGAAATA | 10 |

In some embodiments, an interfering oligonucleotide in accordance with the present invention has a sequence that is at least 50%, 55%, 60%, 65%, 70%, 75%, 80%, 85%, 90%, 95%, 98%, or 99% identical to any of SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10. In some embodiments, the sequence is selected from SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10, and combinations thereof.

It will be appreciated that an interfering oligonucleotide in accordance with the present invention may be of any appropriate length. For example, in some embodiments, an interfering oligonucleotide is 10-50 nucleotides in length. In some embodiments, an interfering oligonucleotide is 10-30 nucleotides in length. In certain embodiments, an interfering oligonucleotide is 15-40 nucleotides in length. In some embodiments, a suitable siRNA is 16-22 (e.g., 16-21, 16-20, 16-19, 16-18, 17-22, 17-21, 17-20, 17-19, 18-22, 18-21, 18-21, or 18-20) nucleotides in length. In some embodiments, a suitable siRNA is or less than 50, 45, 40, 35, 30, 25, 24, 23, 22, 21, 20, 19, 18, 17, 16 or 15 nucleotides in length. In some embodiments, an interfering oligonucleotide is or more than 10, 12, 14, 16, 18, 20, 22, 24, 26, 28, 30, 32, 34, 36, 38, 40, 42, 44, 46, 48, or 50 nucleotides in length

“Percent (%) nucleic acid sequence identity” with respect to the nucleotide sequences identified herein is defined as the percentage of nucleotides in a candidate sequence that are identical with the nucleotides in the reference sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology, 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

Chemical Modifications

RNA molecules, including the interfering oligonucleotides described herein, may be chemically modified to increase intracellular stability and half-life. Possible modifications include the addition of flanking sequences at the 5′ and/or 3′ ends of the molecule or the use of phosphorothioate (also known as thiophosphate) linkages rather than phosphodiesterase linkages within the backbone of the molecule. In addition, one or more ribose groups may be modified to add a methyl moiety to the 2′-OH to form a 2′-methoxy moiety (referred to as 2′O-methyl-modified). Also, the 2′-OH moiety can be linked to the or 3′ or 4′-carbon of ribose by a methylene or ethylene linker, typically a methylene linker to the 4′-carbon, to form a “locked nucleic acid” (see WO 98/39352 and WO 99/14226, the contents of which are incorporated herein by reference).

In certain embodiments, chemical modification also includes the use of nontraditional bases such as inosine, queosine, and wybutosine, as well as acetyl-, methyl-, thio-, and other similarly modified forms of adenine, cytidine, guanine, thymine, and uridine, which are not as easily recognized by endogenous endonucleases. Examples of modified bases include uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; and O- and N-alkylated nucleotides, e.g., N6-methyl adenosine.

In certain embodiments, the sugar moiety can be modified, typically at the 2′-OH of ribose. Examples of such modifications include instances where the 2′ OH-group is replaced by a group selected from H, OR, R, halo, SH, SR, NH₂, NHR, NR₂ or ON, where R is C₁-C₆ alkyl, alkenyl or alkynyl and halo is F, Cl, Br or I.

Further, chemical modification can encompass modified backbones such as morpholino and/or further non-natural internucleoside linkages such as siloxane, sulfide, sulfoxide, sulfone, sulfonate, sulfonamide, and sulfamate; formacetyl and thioformacetyl; alkene-containing; methyleneimino and methylenehydrazino; amide, and the like.

One or more nucleotides (or linkages) within the sequences described herein can be modified. For example, a 20-mer oligonucleotide may contain 1, 2, 3, 4, 5, 6, 7, 8, 9, 10, 11, 12, 13, 14, 15, 16, 17, 18, 19 or 20 modified nucleotides. In certain embodiments, a modified oligonucleotide will contain as few modified nucleotides as are necessary to achieve a desired level of in vivo stability and/or bioaccessibility while maintaining cost effectiveness.

### Pharmaceutical Compositions

The present invention further provides pharmaceutical compositions comprising therapeutically active ingredients in accordance with the invention (e.g., interfering oligonucleotides, small molecules, or combinations thereof), together with one or more pharmaceutically acceptable excipients. Such pharmaceutical compositions may optionally comprise one or more additional therapeutically-active substances.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention. In some embodiments, a pharmaceutical formulation will comprise one or more active ingredients and dimethyl sulfoxide (DMSO).

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Administration

Inventive methods of the present invention contemplate single as well as multiple administrations of a therapeutically effective amount of the therapeutic agents described herein. Therapeutic agents (e.g., interfering oligonucleotides, small molecules, or combination thereof) can be administered at regular intervals, depending on the nature, severity and extent of the subject's condition. In some embodiments, a therapeutically effective amount of the therapeutic agents of the present invention may be administered intravenously, orally, and/or transdermally periodically at regular intervals (e.g., once every year, once every six months, once every five months, once every three months, bimonthly (once every two months), monthly (once every month), biweekly (once every two weeks), weekly).

In some embodiments, delivery is by intravenous administration. In other embodiments, administration can be subcutaneous, intramuscular, parenteral, transdermal, or transmucosal (e.g., oral or nasal).

In some embodiments, provided interfering oligonucleotide compounds may be administered to mammals by various methods through different routes as described herein. For example, they can be administered by intravenous injection. See Song et al., Nature Medicine, 9:347-351 (2003). They can also be delivered directly to a particular organ or tissue by any suitable localized administration methods. Several other approaches for delivery of interfering oligonucleotides, such as siRNA, into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002). Alternatively, they may be delivered encapsulated in liposomes, by iontophoresis, or by incorporation into other vehicles such as hydrogels, cyclodextrins, biodegradable nanocapsules, and bioadhesive microspheres.

As described above, the term “therapeutically effective amount” is largely determined based on the total amount of the therapeutic agent contained in the pharmaceutical compositions of the present invention. A therapeutically effective amount is commonly administered in a dosing regimen that may comprise multiple unit doses. For any particular composition, a therapeutically effective amount (and/or an appropriate unit dose within an effective dosing regimen) may vary, for example, depending on route of administration or on combination with other pharmaceutical agents.

It is to be understood that for any particular subject, specific dosage regimens should be adjusted over time according to the individual need and the professional judgment of the person administering or supervising the administration of the enzyme replacement therapy and that dosage ranges set forth herein are exemplary only and are not intended to limit the scope or practice of the claimed invention.

The invention will be more fully understood by reference to the following examples. They should not, however, be construed as limiting the scope of the invention. All literature citations are incorporated by reference.

### EXAMPLES

Unless otherwise described in a particular Example, the reagents, protocols and constructs used in each Example are as described below. In each case, one of skill in the art will recognize that variations of particular reagents or procedures would be acceptable equivalents and it is contemplated that these alternatives are considered as part of the present description. The Examples below are intended only to provide specific exemplary embodiments, and are not limiting.

### Reagents and Antibodies

Chemical reagents used in the following Examples were of analytical grade or higher and purchased from Sigma unless otherwise specified. Cell culture media were from Invitrogen. Polyclonal antibodies against human FAPP2, Bet3, cPLA2IVa, and GM130 were raised in rabbits using glutathione S-transferase fusion proteins as immunogens. All were affinity purified on their corresponding immunogens. The anti-ts045-VSVG clone P5D4, the anti-Flag M5 anti-HA monoclonal antibodies, and the anti-rabbit and anti-mouse IgG Cy3-conjugated antibodies were from Sigma. The Alexa 488-conjugated Cholera toxin B Fragment was from Invitrogen. The Cy3-conjugated Shiga toxin B Fragment was prepared as described²⁰. The mouse monoclonal antibody against GM3 (clone 2590) was from Cosmo Bio Co. Sheep polyclonal antibodies against TGN46 were from AbD Serotech. The Alexa 488 goat anti-mouse and anti-rabbit IgG (H1L) antibodies were from Molecular Probes. All unlabelled purified lipids were from Avanti Polar Lipids. ³H-sphingosine was from PerkinElmer. Stock solutions of GSLs were prepared in chloroform/methanol (2:1 by volume) and of other lipids in hexane/2-propanol (3:2 by volume). Lipid solutions were stored in the dark at −20° C. and warmed to room temperature before use.

### Cell Culture

HeLa, Meb4, GM95, HepG2, HK2, COS7 and MDCK cells were grown and transiently transfected by TransIT-LT1 (Mirus Bio) as described in¹. Stably-expressing HeLa-GM3S cells were obtained after transfection of the 3XHA-GM3S coding plasmid and selection in the presence of G418 (Invitrogen) and screening of monoclonal colonies by indirect immunofluorescence.

### Measurement of GSLs

Metabolic labelling with ³H-sphingosine or ¹⁴C-galactose, GSL extraction and high-performance thin-layer chromatography and analysis were performed as described in¹ and⁶.

### GSL and Transport Measurements

Metabolic labelling with ³H-sphingosine, immunofluorescence and immunoelectron microscopy studies for subcellular protein localization assessments, were performed as described in¹. Transport of the temperature-sensitive mutant of VSVG (ts045-VSVG) was assessed as described previously²¹. Protein purification, and fluorescence studies were performed as described in^{22,23}.

### Immunofluorescence and Morphometric Analysis

All immunofluorescence experiments were performed as described previously^{1,14}. Images are confocal optical slices obtained using an LSM 710 (Zeiss) confocal microscope. Colocalization analysis was performed as described in¹⁴ or by using an object-based colocalization method included in the JACoP v2.0 application for ImageJ¹⁶. In brief, individual mini-stacks in nocodazole-treated cells were considered as objects, whose mass-center position was calculated after segmentation, the perfect coincidence of mass-center positions for two distinct labelling (i.e. Gb3S/TGN46, or GM3S/TGN46) in a single ministack was considered as a positive colocalization event.

### Immunoelectronmicroscopy

Immunoelectronmicroscopy was performed in transfected HeLa, Meb4, and GM95 cells as described previously¹.

### Histological Analysis

Histological and immunofluorescence microscopy and X-Gal analysis were performed as described previously²⁴.

### Plasmids and Constructs

Several of the constructs used in the following Examples were obtained from HeLa RNA by RT-PCR and cloning into appropriate vectors. In brief, total RNA was isolated from HeLa cells; RT-PCR was performed using a poly dT oligo as a primer. The cDNA obtained was used as a template for PCR, using as primers:

For Gb3S
(SEQ ID NO.: 20)
5′-GTTGAATTCGATCTGGGGATACCATGTCC-3′
(SEQ ID NO.: 21)
5′-CACCTCGAGCAAGTACATTTTCATGGCCTC-3′
For GM3S
(SEQ ID NO.: 22)
5′-CAGGAATTCAGAATGAGAAGGCCCAGCTTGTTA-3′
(SEQ ID NO.: 23)
5′-AACGCGGCCGCTGAAATTCACGATCAATGCCTCCA-3′
For LCS
(SEQ ID NO.: 24)
5′-ATAGAATTCTGGCTGCAGCATGCGCGC-3′
(SEQ ID NO.: 25)
5′-CGCGATATCAAGTACTCGTTCACCTGAGCCA-3′

The PCR products were cloned into a linearized pCR2.1 vector, and processed for automatic sequencing. All of the cloned sequences matched the sequence reported in databases for human Gb3S (AF513325) (SEQ ID NO.: 26), GM3S (AY152815.2) (SEQ ID NO.: 27), and LCS ((B4GALT5) (SEQ ID NO.: 28); (NM_004776) (SEQ ID NO.: 29)), respectively. The DNAs corresponding to the various coding sequences were then subcloned into EcoRI/XhoI (Gb3S); EcoRI/NotI (GM3S); EcoRI/EcoRV sites of p3XHA or p3XFLAG.

GFP-FAPP2-wt and W407A constructs were obtained as described in ; GFP-diFAPP2PH-wt and E50A were obtained as follows:
GFP-FAPP2-wt DNA was used as a template for two distinct PCR reactions using as primers:

PCR a)
(SEQ ID NO.: 30)
5′-TCTGAATTCATGGAGGGGGTGCTGTACA-3
(SEQ ID NO.: 31)
5′-TATGGTACCGAGCAAGCAAGCCTTGGCTGATCCC-3
PCR b)
(SEQ ID NO.: 32)
5′-TATGGTACCTTGCTGGAGGGGGTGCTGTACAAGTG-3
(SEQ ID NO.: 33)
5′-TCACTCGAGTTAGCAAGCCTTGGCTGATCC-3

Products from PCR a) were subcloned into EcoRI/KpnI sites of vector pEGFP-C1 to obtain construct pEGFP-FAPP2PH. Subsequently, products from PCR b) were subcloned into KpnI/XhoI sites of the pEGFP-FAPP2PH construct to obtain GFP-diFAPP2PH-wt. A similar procedure was applied to obtain the GFP-diFAPP2PH-E50A mutant using as a template GFP-FAPP2-E50A DNA. GFP-FAPP2-E50A was obtained from GFP-FAPP2-wt by site-directed mutagenesis using the primers:

(SEQ ID NO.: 34)
5′-GAGCATACAAATGGCAGTCTGTGCAATTCAAGTTCATTCTGTAG-3′
(SEQ ID NO.: 35)
5′-CTACAGAATGAACTTGAATTGCACAGACTGCCATTTGTATGCTC-3′

### Statistical Analysis

For statistical analysis, two-tailed Student t-tests were applied to the data unless otherwise specified. A single asterisk=P<0.05; two asterisks=P<0.01; three asterisks=P<0.005.

### Example 1

### FAPP2 Gene Ablation in Mice

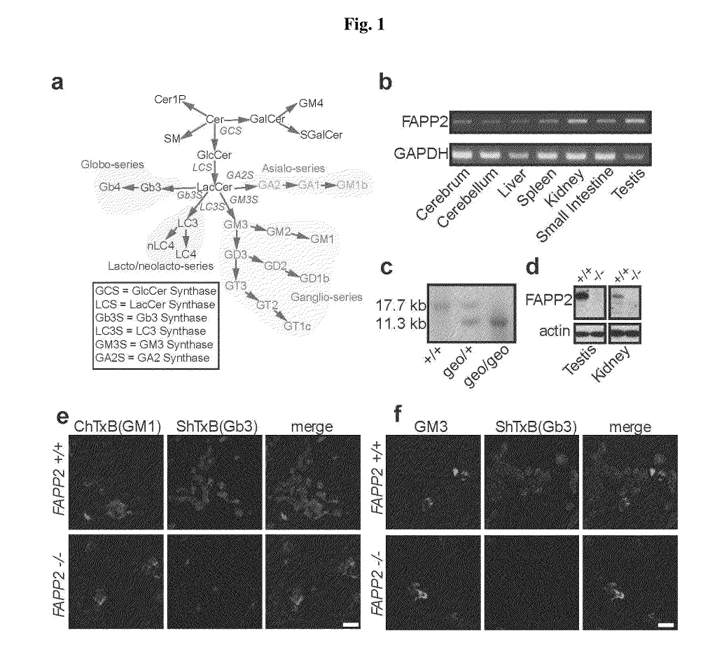

Complex glycosphingolipids (GSLs), which play important roles in cell signaling, adhesion, proliferation and differentiation², are synthesized in the Golgi complex from a common precursor, glucosylceramide (GlcCer). GlcCer is synthesized from ceramide (Cer) by GlcCer synthase (GCS) at the cytosolic leaflet of early Golgi membranes^{3,4}. Upon translocation to the luminal leaflet, GlcCer is galactosylated to lactosylceramide (LacCer) which can then be converted into different series of complex GSLs in later Golgi compartments (FIG. 1a)⁵. GlcCer can be transported through the Golgi complex via membrane trafficking and via non-vesicular transfer due to the action of the cytosolic GlcCer-transfer-protein FAPP2, which fosters complex GSL synthesis^{1,6}. However, the respective roles of the non-vesicular and vesicular transport of GlcCer remain to be defined.

To address this question, FAPP2 knockout (FAPP2−/−) mice were generated as described below and the consequences of FAPP2 gene ablation in mice were analysed (FIGS. 1b-d, and FIG. 8a).

### Establishing FAPP2 Knockout Mice

All animal procedures were performed in accordance with the guidelines of the Animal Care and Experimentation Committee of Gunma University, and all animals were bred in the Institute of Animal Experience Research of Gunma University. We used a previously described knockout system²³ to generate the FAPP2−/− mice. Briefly, the FAPP2 gene was isolated from a mouse genomic BAC library derived from the 129Sv/J mouse strain (RPCI-22: Children's Hospital, Oakland Research Institute). An FRT-flanked SA-IRES-β-geo-polyA cassette was introduced into intron 4 and a loxP site was introduced into intron 3 in the FAPP2 targeting vector (FIG. 8). Recombinant ES cell clones were identified and heterozygous mice (FAPP2geo/+) were generated. By crossing the geo/+ mice with transgenic mice that ubiquitously express Flp recombinase, the SA-IRES-β-geo-polyA cassette is expected to be excised (flox/flox) and the expression of the gene will be recovered. We used the resulting flox/flox mice to generate conditional FAPP2 knockouts by crossing them with Cre transgenic mice.

The following primers were used for genotyping by PCR analysis:

(Primer 1)
(SEQ ID NO.: 11)
5′-GTGCAGGCTGATACGATACTGCAGA-3′,
(Primer 2)
(SEQ ID NO.: 12)
5′-GGATCAAGGCGATGACGTGGATTTC-3′,
(Primer 3)
(SEQ ID NO.: 13)
5′-CCGTACAGTTCCACAAAGGCATCCT-3′,
(Primer 4)
(SEQ ID NO.: 14)
5′-TGGCTGCAGAGCCTTGCTGGTAATG-3′,
(Primer 5)
(SEQ ID NO.: 15)
5′-GTCCCCGGTGATGCTGTGATTGTGA-3′.

Primers 1 (SEQ ID NO.: 11) and 2 (SEQ ID NO.: 12) detected the FAPP2 wild-type allele, primers 1 (SEQ ID NO.: 11) and 3 (SEQ ID NO.: 13) detected the geo allele of FAPP2, and primers 4 (SEQ ID NO.: 14) and 5 (SEQ ID NO.: 15) detected the null allele of FAPP2.

Total RNA was extracted from organs from adult mice by a phenol/chloroform extraction procedure using RNAiso (Takara). 3 μg of total RNA was primed with oligo(dT) to synthesize first-strand cDNA with reverse transcriptase. The primers used for PCR were as follows:

FAPP2 sense primer:
(SEQ ID NO.: 16)
5′-CTCGCATGGACCTCATCATC-3′,
FAPP2 antisense primer:
(SEQ ID NO. 17)
5′-GATGCTGCAATCCACCTCTG-3′,
GAPDH sense primer:
(SEQ ID NO.: 18)
5′-ACCACAGTCCATGCCATCAC-3′,
GAPDH antisense primer:
(SEQ ID NO.: 19)
5′-TCCACCACCCTGTTGCTGTA-3′.

To confirm that FAPP2 is not expressed in the FAPP2−/− mice, Western blots of cellular extract from FAPP2−/− mice using FAPP2-specific antibodies were performed. FIG. 1d shows that FAPP2 proteins were not detected in crude extracts from FAPP2−/− mice.

### Example 2

### FAPP2 Depletion Selectively Inhibits Synthesis of C12-BODIPY-Gb3

FAPP2 knockout (FAPP2−/−) mice showed no overt phenotype. However the visualization of GSL in the kidney, where FAPP2 is highly expressed (FIGS. 1b and 8b), highlighted a specific decrease in one class of GSL. Visualization of the distribution of GSLs using the Shiga toxin B fragment (ShtxB) that binds Gb3⁸, the Cholera toxin B fragment (ChtxB) that binds GM1⁹, and anti-GM3 antibodies confirmed the previously reported distribution of Gb3 in the mouse kidney^{10,11} and, in agreement with the biochemical measurements, showed a selective reduction of Gb3 staining in the FAPP2−/− kidneys (FIGS. 1e, f). Similar results were obtained in primary kidney tubular cells isolated from FAPP2−/− mice (FIGS. 9a, b, d).

FAPP2, in line with its rather recent evolutionary appearance coincident with the divergence of multiple GSL branches from LacCer¹², selectively controls one branch of GSLs in vivo (i.e. globosides, FIGS. 1a, e, f). Moreover, the lack of an overt phenotype in FAPP2−/− mice closely recalls the lack of a phenotype in Gb3 synthase (Gb3S) KO mice¹¹. Without wishing to be bound by any particular theory, the present invention proposes the lack of a phenotype in Gb3S KO mice may be due to compensatory activities by other GSLs and/or to the dependence on adequate stimuli for the phenotypes to manifest.

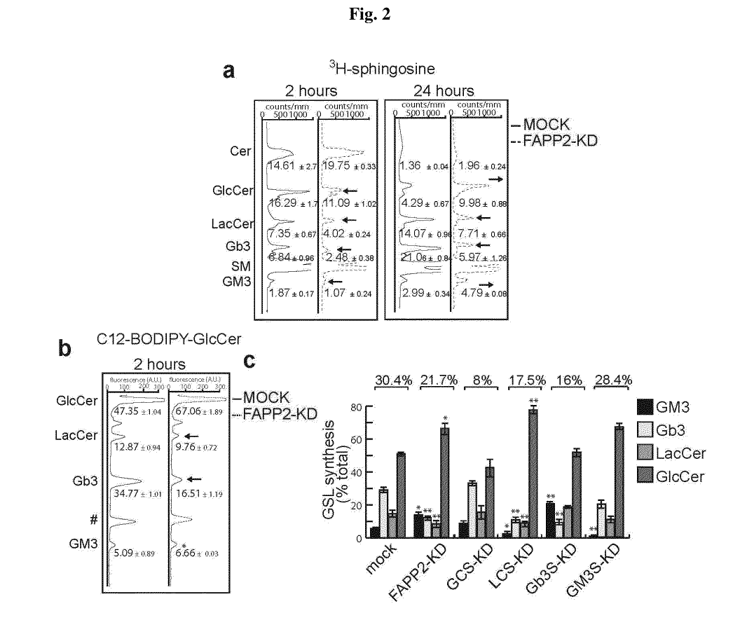

A detailed analysis of the different GSL species was performed, looking at newly synthesized GSLs (FIGS. 2 and 11) in ³H-sphingosine-labelled HeLa cells. FAPP2 depletion inhibited the synthesis of ³H-LacCer and ³H-Gb3 at all time points (FIGS. 2 and 11a). FAPP2 KD, however, also inhibited the synthesis of ³H-GlcCer at early time points, and also lowered, as a consequence, the levels of ³H-GM3 (FIGS. 2a and 11a). Without wishing to be bound by any particular theory, it is thought inhibition of ³H-GlcCer synthesis was possibly due to an inhibition-by-product effect of the locally accumulated non-labelled GlcCer on GlcCer synthase¹.

To circumvent changes in complex GSLs that might be secondary to the inhibition of GlcCer synthesis, GlcCer synthesis was bypassed by labelling the cells with C12-BODIPY-GlcCer. As shown in FIG. 2b, FAPP2 depletion selectively inhibited the synthesis of C12-BODIPY-Gb3 but not of C12-BODIPY-GM3, indicating that the decrease in Gb3 but not in GM3 synthesis is the direct consequence of FAPP2 depletion. Systematic silencing of enzymes involved in GSL biosynthesis (FIGS. 1c, 11b and c) highlighted that the GSL profile induced by FAPP2 silencing was similar to that induced by LacCer Synthase (LCS) silencing in terms of a decrease in LacCer, but they differed in their effects on downstream GSL species since LCS KD induced a uniform decrease in Gb3 and GM3 while FAPP2 KD selectively decreased Gb3 synthesis. These results indicated that the GlcCer transported via FAPP2 feeds a pool of LacCer specifically destined to globoside (i.e. Gb3) synthesis.

### Example 3

### Flux Analysis and Mathematical Modelling of GSL Synthesis

Dynamic assessment of GSL metabolic fluxes followed by mathematical modelling corroborated the data described in Example 2 (FIG. 12). On average, the effect of FAPP2 depletion was more marked on the newly synthesized pool (FIG. 2b) than on the steady state levels of Gb3 (FIG. 1 and FIG. 2a) possibly due to the contribution of GSL salvage pathways to the steady state levels of Gb3.

To assess the SL metabolic fluxes, FAPP2-KD HeLa cells and mock (HeLa cells treated with transfection vehicle)-treated HeLa cells were pulsed for 2 h with ³H-sphingosine followed by a chase for 0, 2, 6, and 24 h (FIG. 11a). The rates of Cer consumption and SM production in FAPP2-KD cells were comparable to control cells, although the overall SM levels were significantly higher at all time points. As previously reported¹, GlcCer levels were lower in FAPP2-KD cells at the early time points, possibly due to a product-inhibition effect of GlcCer on GCS as a consequence of impaired GlcCer consumption that, indeed, accumulates over time. Surprisingly, while Gb3 and LacCer synthesis were inhibited in FAPP2-KD cells, GM3 synthesis was not.

The above analysis indicated that FAPP2 depletion induces a complex rearrangement of GSL metabolism, raising the question as to whether this rearrangement was a direct consequence of impaired GlcCer transport or whether additional effects should be envisaged. To address this question a mathematical model was built based on the experimental data shown in FIG. 11a. GSL reactions were modelled as first-order reactions, by means of Ordinary Differential Equations (ODE), following the law of mass action. Different conditions were considered in which FAPP2 depletion alternatively affected the reaction rates (k₁ to k₅) corresponding to the synthetic steps leading from Cer to SM (k₁), from SM to Cer (k_{1R}), from Cer to GlcCer (k₂), from GlcCer to LacCer (k₃), from LacCer to Gb3 (k₄) or to GM3 (k₅). Additionally, k_{3A} and k_{3B} were introduced as reaction rates leading to the two pools of LacCer (LacCerA and LacCerB) destined for the synthesis of Gb3 and GM3, respectively (FIG. 12a). The ODEs for GSL conversion read:

$$\frac{d Cer}{d t} = - {k}_{1} Cer + {k}_{1 R} SM - {k}_{2} Cer$$

$$\frac{d GlcCer}{d t} = {k}_{2} Cer - ( {k}_{3} + {k}_{3 A} + {k}_{3 B} ) GlcCer$$

$$\frac{d {LacCer}_{A}}{d t} = {k}_{3 A} GlcCer - {k}_{4} {LacCer}_{A}$$

$$\frac{d {LacCer}_{B}}{d t} = {k}_{3 B} GlcCer - {k}_{5} {LacCer}_{B}$$

$$\frac{d LacCer}{d t} = {k}_{3} GlcCer$$

$$\frac{d {Gb}_{3}}{d t} = {k}_{4} {LacCer}_{A}$$

$$\frac{d {GM}_{3}}{d t} = {k}_{5} {LacCer}_{B}$$

$$SM = {Cer}_{TOT} - Cer - LacCer - {LacCer}_{A} - {LacCer}_{B} - {Gb}_{3} - {GM}_{3}$$

The reaction rates (k₁-k₅) were optimized using the MATLAB toolbox SBtoolbox2 in combination with SBPD [www.sbtoolbox2.org] and the local optimization method “SBsimplex”. The best fit was obtained by minimizing an objective function, or Cost Function (CF), here chosen as the square of distances between experimental and simulated data points. In the initial simulation, all reaction rates were required to have the same value for mock-treated and FAPP2-KD cells (null hypothesis, N in FIG. 12b). In subsequent simulations, the reaction rates were allowed to vary one at a time (from 0.01 to 10 fold with respect to the value assigned in N) between mock-treated and FAPP2-KD cells (FIG. 12b). The lowest CF (0.019) was obtained when FAPP2 depletion was modelled to affect k_{3A}, i.e. GlcCer to LacCerA conversion. Thus, a reduction in k_{3A} (from 0.045 to 0.0063) is the change that best describes the overall effects of FAPP2-depletion on GSL metabolic fluxes. The simulation corresponding to this condition is shown in FIG. 12c.

### Example 4

### FAPP2 Drives the Transfer of GlcCer from the cis-Golgi the TGN

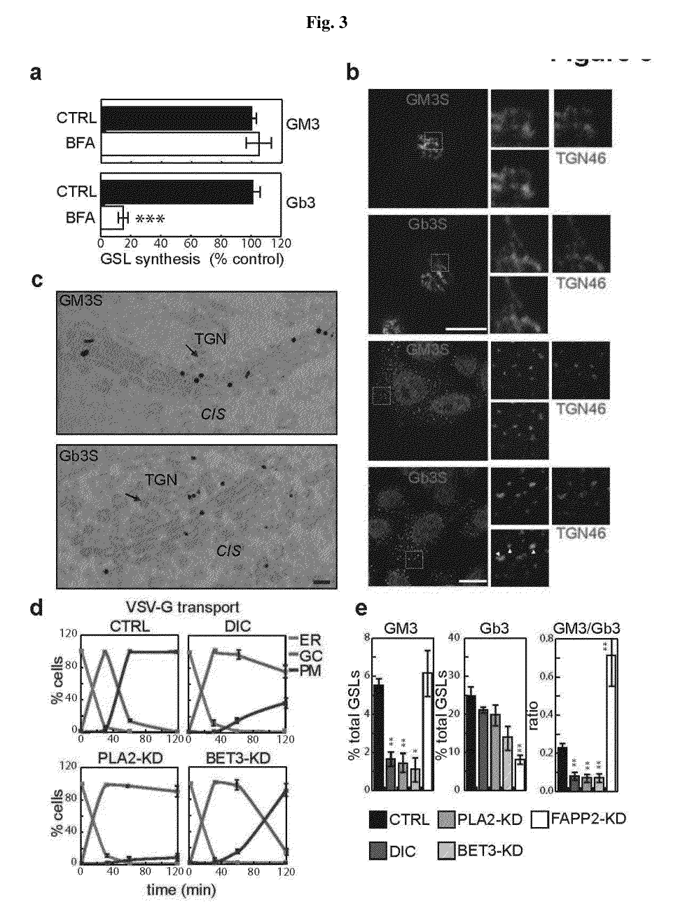

To search for the mechanisms responsible for the different sensitivities of Gb3 and GM3 synthesis to FAPP2 depletion, sub-Golgi distribution of Gb3S and of GM3 synthase (GM3S) were studied by combining two independent approaches⁷. First, synthesis of Gb3 and GM3 was measured in cells treated with BFA, a fungal toxin that redistributes the Golgi cisternae (but not the TGN) into the endoplasmic reticulum (ER) (generating an ER-Golgi intermixed compartment), interrupts vesicular trafficking from this intermixed compartment to the TGN¹³, and releases FAPP2 from Golgi membranes¹⁴. BFA treatment decreased the synthesis of Gb3 but not that of GM3, indicating that the major fraction of endogenous Gb3S (but not of GM3S) resides in the TGN and thus remains segregated from its substrates that are synthesized in the BFA-induced intermixed ER-Golgi compartment (FIG. 3a). Second, due to the unavailability of antibodies suitable for the immunolocalization of the endogenous enzymes, distribution of HA-tagged forms of GM3S and Gb3S was analysed (FIGS. 3b, c).

### Localization of LCS and of GM3S in HeLa Cells

Two distinct LacCer pools destined for GM3 or Gb3 synthesis were analysed to determine if they were produced by the same LacCer synthase (LCS) or by different enzymes. It has been reported that both the B4GALT5 and B4GALT6 genes encode LCSs³¹, so a first step was to define the molecular nature of LCS in HeLa cells. Expression of the B4GALT5 or the B4GALT6 gene products was silenced in HeLa cells (FIG. 15a) and effect of these treatments were assessed on GSL levels by ³H-sphingosine pulse labelling for 24 hours. As shown in FIG. 15b, B4GALT5 KD induced a nearly complete inhibition of LacCer and downstream GSL synthesis (both Gb3 and GM3, FIG. 2c, FIG. 15b) while B4GALT6 KD did not (FIG. 15b). These results indicated that the two pools of LacCer are generated by the same LCS, B4GALT5, which, as assessed in immunoelectron microscopy (IEM) studies, is present both in the Golgi cisternae and in the TGN in cells expressing low levels of 3XFlag-B4GALT5 (FIGS. 15c, d).

Cells expressing HA-GM3S were classified into low and high expressing cells (FIGS. 16a and b) and low-expressing cells were chosen for morphological analysis of GM3S distribution (FIGS. 3b, c). HA-GM3S localized mainly in the Golgi cisternae when expressed at low levels (FIGS. 3b, c), but also localized at the TGN when expressed at high levels (FIGS. 16a and b). This observation was exploited to challenge the hypothesis that the ability of FAPP2 to operate a cis-to-TGN shunt of GlcCer is required to feed a LacCer pool at the TGN that is used by TGN-resident, GSL-synthesizing enzymes. To investigate whether GM3 synthesis required FAPP2, a stable HeLa cell clone was isolated expressing high levels of 3XHA-GM3S (HeLa-GM3S); a significant fraction of GM3S resided in the TGN. HeLa-GM3S produced approximately 7 times more GM3 than parental HeLa cells at the expense of Gb3 production (FIG. 16c), consistent with earlier reports¹⁵. In agreement with the ectopic localization of GM3S at the TGN seen by IEM in these cells, GM3 synthesis became sensitive to BFA treatment (FIG. 16d), and interestingly, also to FAPP2 KD (FIG. 16e), thus supporting these data that the ability of FAPP2 to transfer GlcCer to the TGN is required for the activity of GSL synthesizing enzymes residing in this compartment.

Gb3S was enriched in the TGN¹⁶ while GM3S was enriched in the Golgi cisternae. Moreover, consistent with its effect on the synthesis of GSL (FIG. 3a), BFA redistributed GM3S, but not Gb3S, to the ER (FIG. 13). These data were confirmed in HeLa, HepG2, MDCK, MEF and HK2 cell lines (FIG. 14).

The synthesis of globosides at the TGN relies on the non-vesicular transport of GlcCer operated by FAPP2 while the synthesis of GM3 in the Golgi cisternae does not, eliciting the question as to whether GM3 synthesis depends instead on the vesicular transport of GlcCer. To address this question intra-Golgi membrane trafficking¹ was inhibited by treating cells with dicoumarol, by depleting cPLA2¹⁶, or by depleting the TRAPP complex component Bet3, and followed the transport of the reporter protein ts045 VSV-G (VSVG)¹⁴. These treatments suppressed the intra-Golgi progression of VSVG and strongly inhibited GM3 synthesis, but not Gb3 synthesis, thus leading to a decrease in the GM3/Gb3 ratio (FIGS. 3d, e). Transport of the temperature-sensitive mutant of VSVG (ts045-VSVG) was assessed as described previously²¹.

These data support the hypothesis that the vesicular transport of GlcCer feeds a pool of LacCer that is made in the Golgi cisternae and used for the biosynthesis of GM3, while the non-vesicular transport of GlcCer via FAPP2 feeds a pool of LacCer that is made in the TGN and used in this compartment for the biosynthesis of globosides. This hypothesis generated some key predictions: (i) LCS should be present not only in the Golgi cisternae but also in the TGN, (ii) other LacCer derivatives, which, similarly to Gb3, are made at the TGN should depend on FAPP2, and (iii) artificially shifting the localization of GM3S from the Golgi cisternae to the TGN should make GM3 synthesis sensitive to FAPP2 depletion. All of these predictions were verified. Firstly, LCS was found to localize both to the Golgi cisternae and to the TGN (FIGS. 15c, d); secondly, FAPP2 KD in SK-N-MC neuronal cells selectively lowered the synthesis of GA2, which is made in the TGN by the addition of GalNAc to LacCer (FIG. 1a); thirdly, GM3 synthesis, which is normally insensitive, becomes sensitive to FAPP2 depletion when a significant fraction of GM3S is forced to localize at the TGN by overexpression of the enzyme (FIGS. 16b-d).

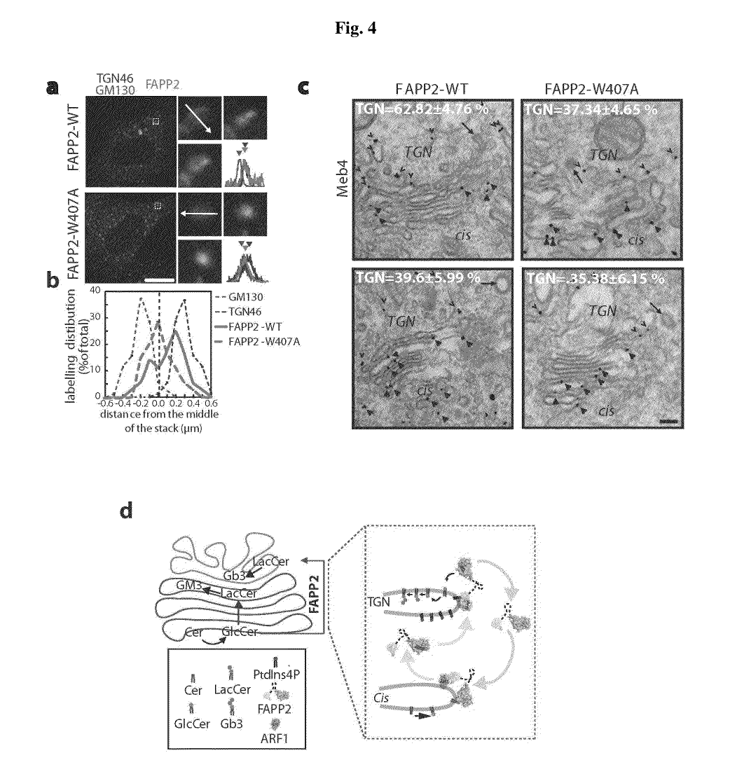

The selective requirement of FAPP2 for GSLs synthesized at the TGN, together with previous observation that cells depleted of FAPP2 fail to concentrate GlcCer at the TGN¹, indicate that FAPP2 drives the transfer of GlcCer from the cis-Golgi, where GlcCer is synthesized, to the TGN and raises the question of how this vectorial transport is sustained. Without wishing to be bound by any particular theory, the present invention proposes that in order to mediate the cis-Golgi-to-TGN transfer of GlcCer, the apo FAPP2 should be preferentially targeted to early Golgi membranes while GlcCer-bound FAPP2 should be preferentially targeted to the TGN. To test this hypothesis, the distribution of FAPP2-wt was compared with that of a single-point mutant of FAPP2, which is unable to bind GlcCer and thus is permanently in an apo form (FAPP2-W407A)¹. While the major fraction of FAPP2-wt localizes at the TGN, the major fraction of the FAPP2-W407A mutant localizes to the Golgi cisternae (FIGS. 4a-c). Moreover, FAPP2-wt failed to localize at the TGN and was present mainly in the Golgi cisternae in a cell line that does not synthesize GlcCer (GM95 cells¹⁸) where FAPP2 is always apo (FIG. 4c). The inability to bind GlcCer and the lack of GlcCer, both of which force FAPP2 into its apo form, compromise the TGN targeting of FAPP2 illustrating that binding of GlcCer positively regulates the targeting of FAPP2 to the TGN.

### Example 5

### Analysis of ARF-Recruiting Activity of the FAPP PH-E50A Domain Mutant

FAPP2 localization at the TGN is determined by its PH domain that coincidentally and independently¹⁹ binds the small GTPase ARF1 and PtdIns4P¹⁴, a phosphoinositide enriched at the TGN²⁰. Single point mutations either in the PtdIns4P¹⁴ or in the ARF-binding site¹⁹ abolish the recruitment of the monomeric PH domain to the Golgi complex (see ref 14 and data not shown), indicating a requirement for both binding sites. Interestingly, however, when these mutations are introduced into tandem forms of the FAPP PH-domain (di-PH), such that the di-PH has two binding sites either for ARF (di-PH-R18L) or for Ptdins4P (di-PH-E50A), the chimeric proteins are now able to localize to the Golgi complex, although with significantly different sub-Golgi distributions (FIG. 17).

The E50A mutation has been shown to impair the binding of the FAPP1-PH domain to ARF1 in vitro¹⁹. The same mutation was introduced in the FAPP2-PH domain to prepare a GFP-FAPP2 PH-E50A expression construct. GFP-FAPP2 PH-E50A was expressed in HeLa cells and then evaluated for its ability to bind ARF1 in intact cells. As a consequence of its ability to bind ARF1 and to compete with ARF-GAP1¹⁴, FAPP-PH, in its tandem form, stabilizes ARF1 on Golgi membranes¹⁴. Tandem forms of GFP-FAPP2 PH-E50A (diFAPP2-PH-E50A) were expressed and verified that, compared to diFAPP2-PH-wt, it had lost its ability to interact with ARF1 also in intact cells (FIG. 17a).

In particular, a mutant FAPP-PH domain with a lower affinity for PtdIns4P and a higher affinity for ARF1 (di-PH-R18L) distributes throughout the Golgi stacks¹⁴, while a mutant FAPP-PH domain with a lower affinity for ARF1 and a higher affinity for PtdIns4P (di-PH-E50A)¹⁹, preferentially localizes to the TGN (FIG. 17c), indicating that, of the two ligands, it is PtdIns4P that dictates the TGN targeting of FAPP2.

Preferential TGN association of GlcCer-bound FAPP2 was analysed to determine, as compared to apo-FAPP2, if GlcCer-bound FAPP2 has a higher affinity for PtdIns4P. GlcCer loading (discussed below and FIG. 18) increased FAPP2 binding to PtdIns4P as measured by Surface Plasmon Resonance (data not shown).

### Example 6

### Assessment of GlcCer loading of FAPP2

The GlcCer loading efficiency was measured by exploiting the presence of a key tryptophan (W407) in the putative FAPP2-GlcCer binding site, which is conserved compared to the glycolipid transfer protein GLTP²¹. As reported for GLTP²⁵ and for the GLTPH domain of FAPP2²¹, GlcCer binding to full length FAPP2 induced a substantial decrease in tryptophan fluorescence intensity along with a shift of tryptophan fluorescence emission maxima towards lower wavelengths. No change was observed with the FAPP2-W407A mutant confirming that the observed effect was due to the quenching of W407-associated fluorescence (FIG. 18a). The procedure described in ²⁵ was applied to calculate the fraction of GlcCer-loaded FAPP2. In brief, the fraction of binding sites (α) occupied by C8-GlcCer was calculated by the equation

α=(F−F0)/Fmax

where F0 and F are the W emission intensities of FAPP2 in the absence and presence of C8-GlcCer, respectively, and Fmax is the emission intensity of the fully liganded FAPP2, i.e. at excess C8-GlcCer. Fmax was determined by plotting 1/(F−F0) vs. 1/L and extrapolating 1/L=0, where L equals the total glycolipid concentration. As a further and independent approach to assess the GlcCer loading of FAPP2, the circular dichroism (CD) spectra of FAPP2 wt and FAPP2-W407A was analzyed upon exposure to GlcCer. GlcCer induced a significant change in the CD profile of full length FAPP2wt, but not of full length FAPP2-W407A (FIG. 18b). These data were in agreement with results obtained with the GLTP homology domain of FAPP2²¹.

Without wishing to be bound by any particular theory, the present invention proposes a “FAPP2 cycle”, wherein apo-FAPP2 associates with the cis-Golgi where it acquires GlcCer, resulting in a higher affinity of FAPP2 for PtdIns4P. FAPP2 then relocates to the PtdIns4P-enriched TGN where it delivers GlcCer (FIG. 4d).

These data establish a new paradigm for the branching of GSL biosynthesis at the Golgi complex whereby two branches receive their common precursor, GlcCer, from two parallel anterograde transport routes (FIG. 4d). These are the vesicular route, which traverses the Golgi cisternae and feeds the LacCer pool used to make GSLs of the ganglio-series, and the non-vesicular route mediated by FAPP2, which delivers GlcCer to the TGN, bypassing the intervening cisternae, and feeds a TGN pool of LacCer converted in loco into GSLs of the globo-series (FIG. 4d and FIG. 1a). In a wider context, these data show how different modes of transporting a cargo through the Golgi complex channel the cargo itself to distinct and otherwise potentially competing glycosylation pathways.

The finding the FAPP2 specifically control the synthesis of Gb3 makes it a candidate target for those conditions characterized by Gb3 accumulation. These are exemplified by Fabry disease linked to mutations in α-galactosidase A, the lysosomal enzyme that degrades Gb3.

### Example 7

### Inhibition of FAPP2 Reduces Accumulation of Gb3 in Cells from Fabry Patients

The present Example demonstrates that FAPP2 inhibition decreases Gb3 accumalation in cells from Fabry patients depleted of α-galactosidase A.

Among other things, the present Example also describes the levels/distribution of Gb3 in fibroblast from Fabry patients using Cy3-shiga toxin.

### Materials and Methods

siRNA Treatments

The siRNAs for human FAPP2 (NM_001197026 or NM_001197026.1) (SEQ ID NO.: 89), GCS (NM_003358) (SEQ ID NO.: 36), Bet3 (NM_014408) (SEQ ID NO.: 37), B4GALT5 (NM_004776) (SEQ ID NO.: 38), B4GALT6 (NM_004775) (SEQ ID NO.: 39), SIAT9/GM3S (AY152815.2) (SEQ ID NO.: 40), A4GALT/Gb3S (NM_017436) (SEQ ID NO.: 4), PLA2 (NM_001199562) (SEQ ID NO.: 42) comprised mixtures of at least 3 siRNA duplexes (Table 3) and were obtained from Dharmacon. HeLa, HK2, HepG2 and MDCK cells were plated at 30% confluence in 12-well plates and transfected with 120-150 μmol of siRNAs with Oligofectamine (Invitrogen) or Dharmafect4 (Dharmacon), in accordance with the manufacturer's protocol. At 72 h after the initial treatment with siRNA, the cells were processed directly. Silencing efficiency was evaluated either by Western blot (FIG. 10) or by q-PCR (FIG. 11) using specific primers (Table 4).

TABLE 3
Sequences of siRNA and shRNA oligonucleotides

| | | SEQ |
|---|---|---|
| Name | sRNA sequence | ID NO: |
| FAPP2.1 | GAGAUAGACUGCAGCAUAU[dT][dT] | 3 |
| FAPP2.2 | GAAUUGAUGUGGGAACUUU[dT][dT] | 4 |
| FAPP2.3 | GAAAUCAACCUGUAAUACU[dT][dT] | 5 |
| FAPP2.4 | CCUAAGAAAUCCAACAGAA[dT][dT] | 6 |
| GSC.1 | GAUAUGAAGUUGCAAAGUA[dT][dT] | 43 |
| GSC.2 | GCGAAUCCAUGACAAUAUA[dT][dT] | 44 |
| GSC.3 | GGACCAAACUACGAAUUAA[dT][dT] | 45 |
| GSC.4 | GAUGCUAGAUUGUUUAUAG[dT][dT] | 46 |
| B4GALT5.1 | GUGAAAUUGGAAUGGAUUA[dT][dT] | 47 |
| B4GALT5.2 | GCUUAACAGUGGAACAAUU[dT][dT] | 48 |
| B4GALT5.3 | GGAAAGUGAUCGCAACUAU[dT][dT] | 49 |
| B4GALT6.1 | CAUAUCUCUUUAUGGUACA[dT][dT] | 50 |
| B4GALT6.2 | CUCCAAUCGAAGACUAUUA[dT][dT] | 51 |
| B4GALT6.3 | GGUAUUCCAAGGAGCGUCA[dT][dT] | 52 |
| SIAT9.1 | CAAUGGCGCUGUUAUUUGA[dT][dT] | 53 |
| SIAT9.2 | GACCAUGCAUAAUGUGACA[dT][dT] | 54 |
| SIAT9.3 | CGGAAGUUCUCCAGUAAAG[dT][dT] | 55 |
| SIAT9.4 | AGGAAUACUGCACGGAUUA[dT][dT] | 56 |
| A4GALT.1 | AGAAAGGGCAGCUCUAUAA[dT][dT] | 57 |
| A4GALT.2 | GGACACGGACUUCAUUGUU[dT][dT] | 58 |
| A4GALT.3 | UGAAAGGGCUUCCGGGUGG[dT][dT] | 59 |
| A4GALT.4 | GCACUCAUGUGGAAGUUCG[dT][dT] | 60 |
| PLA2.1 | GGACAGUCGUUAAGAAGUA[dT][dT] | 61 |
| PLA2.2 | GGAGAAACACUAAUUCAUA[dT][dT] | 62 |
| PLA2.3 | GGAGAAGACUUUCAGACAA[dT][dT] | 63 |
| PLA2.4 | GUACAAGGCUCCAGGUGUU[dT][dT] | 64 |
| Bet3.1 | GAUAACCACUCAUCCCUUA[dT][dT] | 65 |
| Bet3.2 | GGGCAUCACUCCAAGCAUU[dT][dT] | 66 |
| Bet3.3 | GAAAGGAGACGGUGUGACA[dT][dT] | 67 |
| Bet3.4 | GGAAACUGCGGAUGUCAUU[dT][dT] | 68 |
| shFAPP2.1 | CTCTTGTGGCTGAAGAGAGGTCTCAAATT | 7 |
| shFAPP2.2 | TTGGCAGCCTCGATGGTTCCTTCTCTGTG | 8 |
| shFAPP2.3 | CAGTCTGGATCAGACTCAAGTTGCTCTCC | 9 |
| shFAPP2.4 | TCCTGTTAAGATGGATCTTGTTGGAAATA | 10 |

TABLE 4
Sequences of rt-qPCR primer oligonucleotides

| Name | Primer sequence | SEQ ID NO: |
|---|---|---|
| βACTIN(+) | AAGAGCTACGAGCTGCCTGA | 69 |
| βACTINO | GACTCCATGCCCAGGAAGG | 70 |
| HPRT1(+) | TGCTGACCTGCTGGATTACA | 71 |
| HPRT1(-) | CCTGACAAGGAAAGCAAAG | 72 |
| B4GALT5(+) | CAATCGGTGCTCAGGTTTATG | 73 |
| B4GALT5(-+) | GGTTTCACTGTGGTTCAAGTC | 74 |
| B4GALT6(+) | TCCTAAGTCTCCCTCTGGTC | 75 |
| B4GALT6(-+) | ACGTATCTCCCGGAAAACTTC | 76 |
| FAPP2(+) | GGAGGGGGTGCTGTACAAGT | 77 |
| FAPP2(-) | GGACAATATTCCCCCACAGA | 78 |
| A4GALT(+) | GATCCCCACCTCTCTGCAAT | 79 |
| A4GALT(-) | TTGGACATGGTATCCCCAGA | 80 |
| SIAT9(+) | TGGTTATTGGAAGCGGAGG | 81 |
| SIAT9(-) | TCTGAATATCCCTCAACTGGTG | 82 |
| GCS(+) | TTCGGGTTCGTCCTCTTC | 83 |
| GCS(-) | GCTTGCTATAAGGCTGTTTGTC | 84 |

Measurement of Gb3 in Fibroblasts from Fabry Disease (FD) Patients

Fibroblasts from male FD patients were derived from skin biopsies after obtaining the informed consent of patients. Normal age-matched control fibroblasts were available in the laboratory of the Department of Pediatrics, Federico II University of Naples. All cell lines were grown at 37° C. with 5% CO2 in Dulbecco's modified Eagle's medium (Invitrogen, Grand Island, N.Y.) and 10% fetal bovine serum (Sigma-Aldrich, St Louis, Mo.), supplemented with 100 U/ml penicillin and 100 mg/ml streptomycin. The cells were used for the experimental procedures indicated below after 4-6 passages.

### FAPP2KD

The FAPP2 KD was attained in human fibroblasts by treatment with FAPP2-siRNAs for 96 hrs, according to the protocol described in D'Angelo et al., 2013. The sequence of the siRNAs is also specified in Suppl Material of D'Angelo et al. 2013.

TABLE 5
Mutations of Fabry Disease patients

| | | | Fibroblast | |
|---|---|---|---|---|
| | | | alpha-Gal A | |
| | Genotype | | activity | Literature |
| Patient | Exon | Mutation | (nmol/hr/ml) | reference |
| 1 | 6 | p.A288D | 4.86 | Eng et al. 1994 |
| 2 | 4 | Aberrant splicing | 5.16 | Okumiya et al. 1995 |
| | | IVS4 + 5 G > T | | |
| 3 | 4 | Aberrant splicing | 6.5 | Okumiya et al. 1995 |
| | | IVS4 + 5 G > T | | |
| 4 | 3 | c.452delA fsV164X | 4.47 | Not reported |
| 5 | 5 | p.R227Q | 4.48 | Eng et al. 1993; |
| | | | | Benjamin et al. |
| | | | | 2009 |
| 6 | 6 | c.946delG fsV316X | 4.60 | Gal 2010 |

### Example 8

### FRET-Based GlcCer Transfer Assay

The present examples illustrates an exemplary in vitro assay that can be used to screen, test and identify inhibitors of FAPP2 that can be used for drug development.

Specifically, acceptor vesicles (formed by sonication of 1,2-dioleoyl-sn-glycero-3-phosphocholine (DOPC) were suspended in 10 mM NaH₂PO₄ buffer, pH 7.4, containing 1 mM dithiothreitol and 1 mM EDTA) and were incubated with donor vesicles containing BODIPY-labeled GlcCer (1 or 2 mole %, as indicated) and Di1C18 used as quencher (3 mole %), and recombinant FAPP2 protein (at the indicated concentrations).

Recovery of emission intensity at 520 nm (excitation at 485 nm) occurred during protein-mediated transfer of GlcCer from quenched donor vesicles to unquenched acceptor vesicles.

The assay was performed as described in Ref 1 and in Ref 25 using Full-Length (FL) and the inactive FL FAPP2-W407A mutant. The Tryptophan at position 407 has been demonstrated to be crucial for FAPP2 binding activity.

Ultrastructure of Small Unilamellar VesiclesVesicle size and shape were investigated by Electron Microscope using NANOVAN as negative stain. Vesicle sizes ranged from 30 to 40 nm in diameter.

Phlorizin and Dapagliflozin were resuspended in DMSO (100 mM). The assay was conducted at four different concentrations (1 mM, 500 uM, 200 uM, 100 uM). Both FAPP2 WT and FAPP2 W407A were used to highlight possible non-specific effects of the drugs on fluorescence emission. First, mixture that contained FAPP2, acceptor small unilamellar vesicles and drug in buffer were added to 96 well plates, the plate was immediately loaded into the plate reader. Following 2 sec of shaking, the fluorescence emission @520 nm (excitation 485 nm) was measured at 10 sec intervals for 5 mins to calculate the baseline fluorescence. Then, donor vesicles were added to each well and read again for the indicated time. The experiments were repeated at least 3 times and each measurement performed in triplicate.

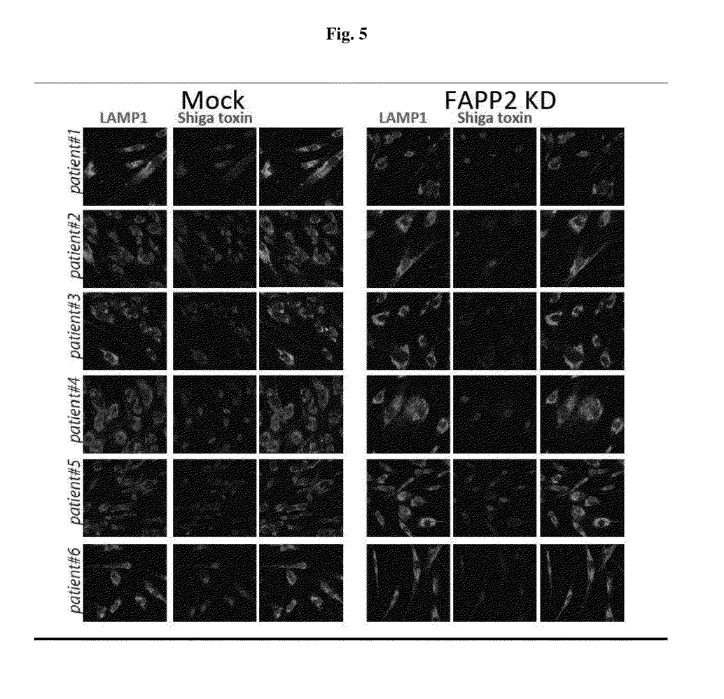

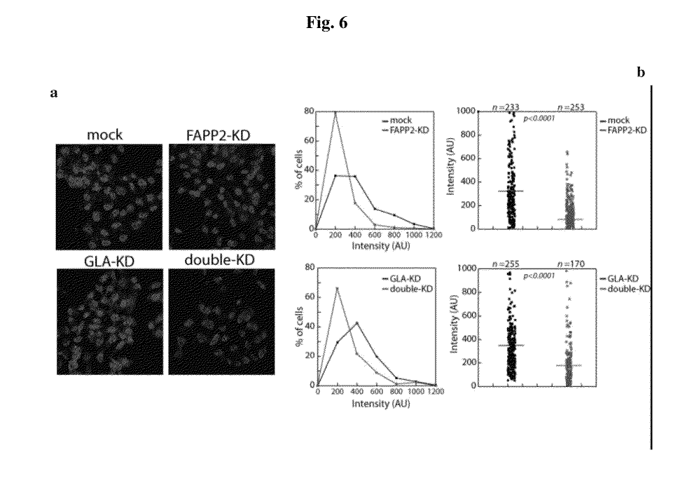

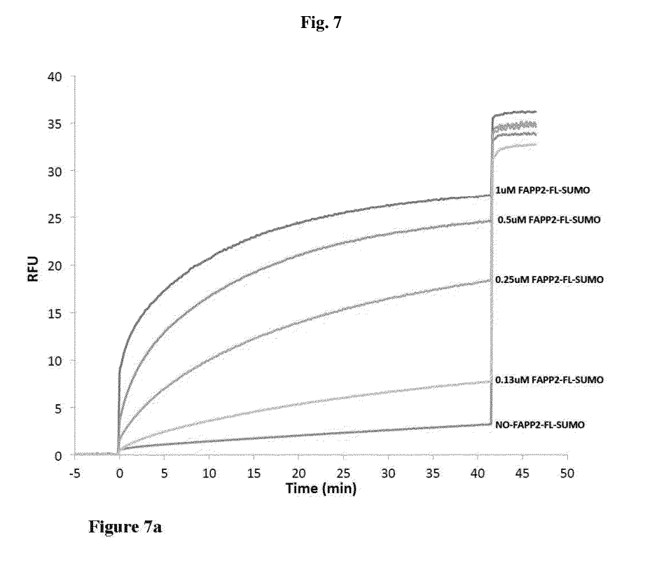

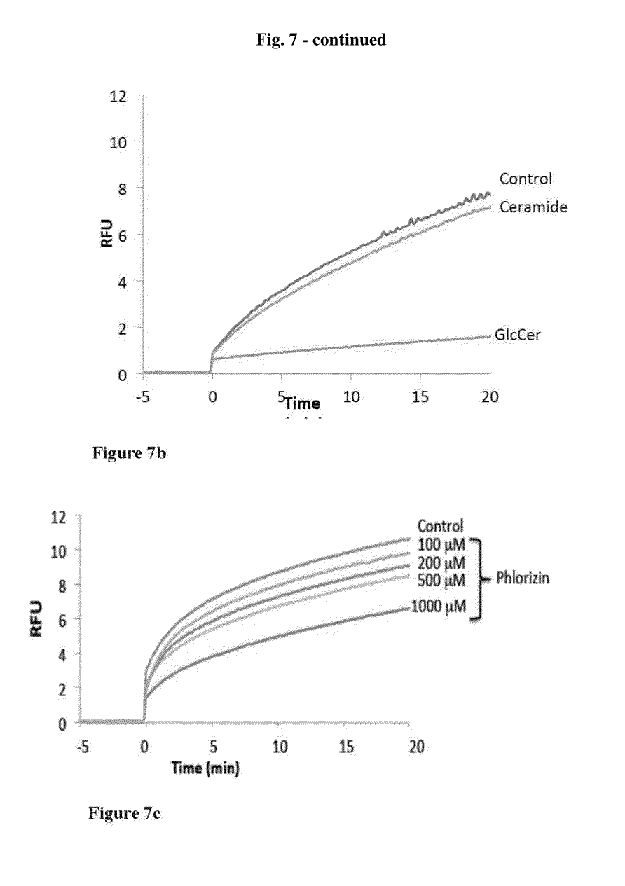

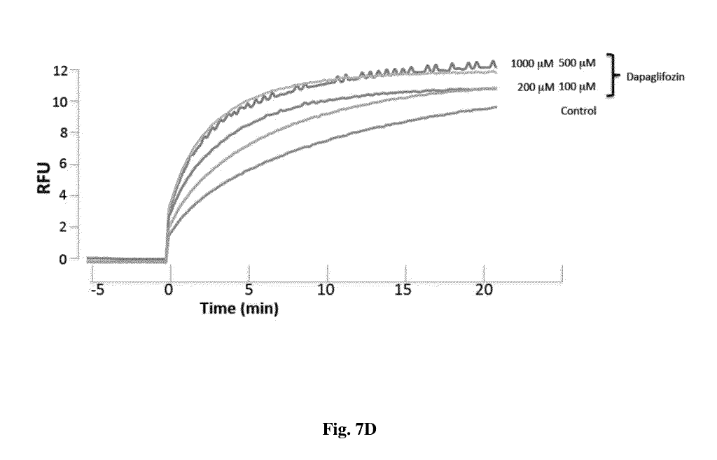

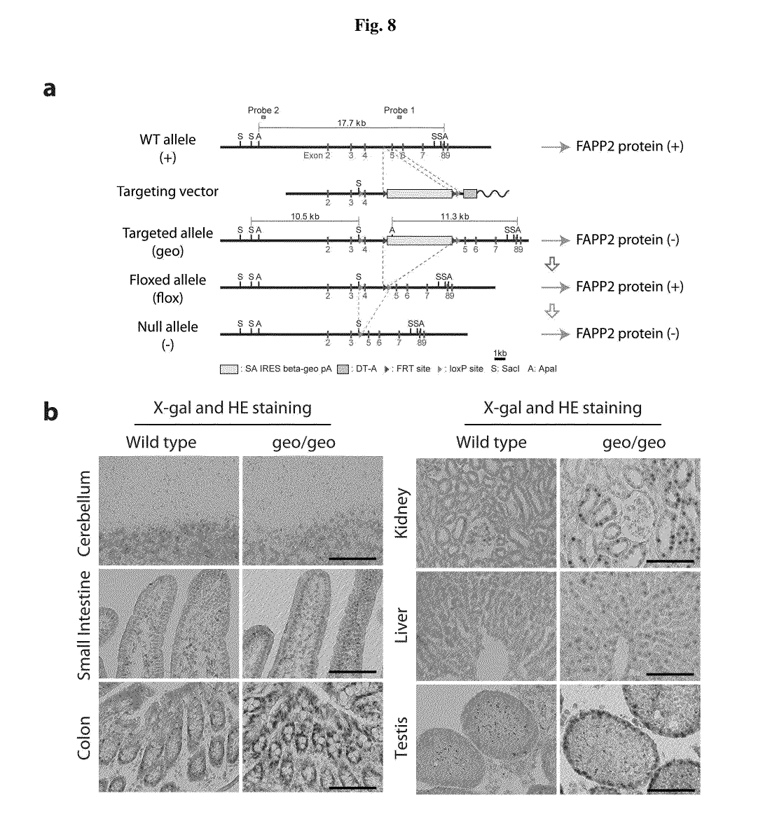

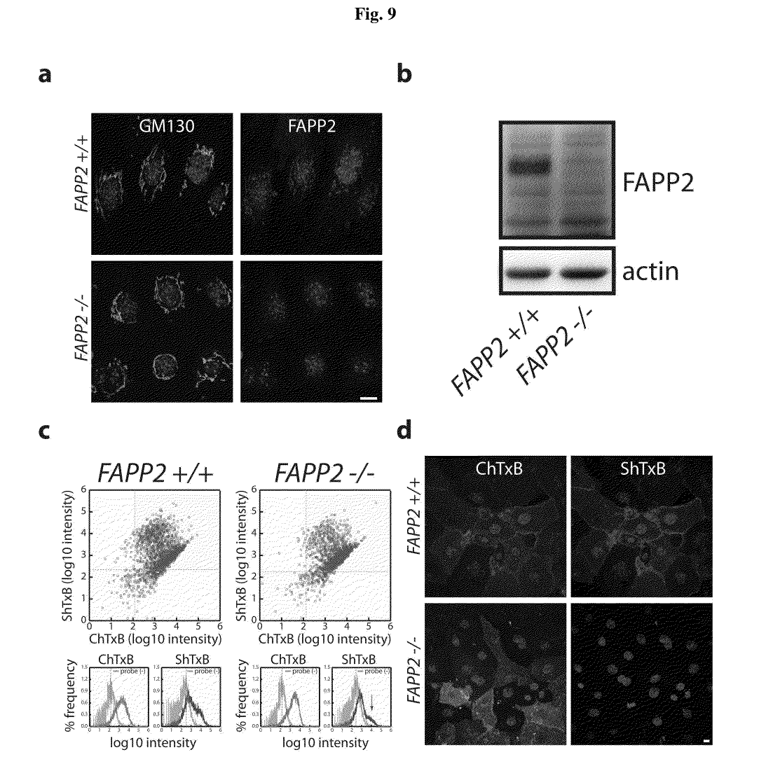

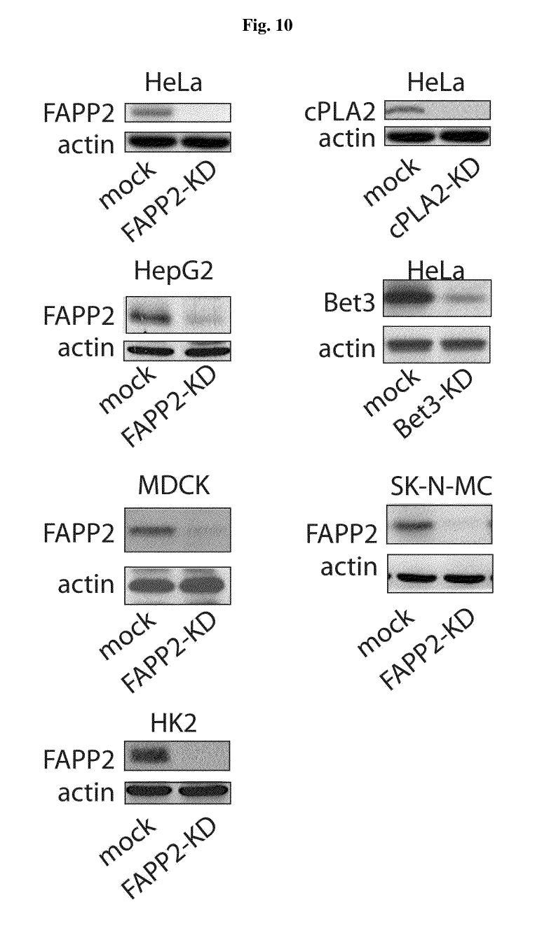

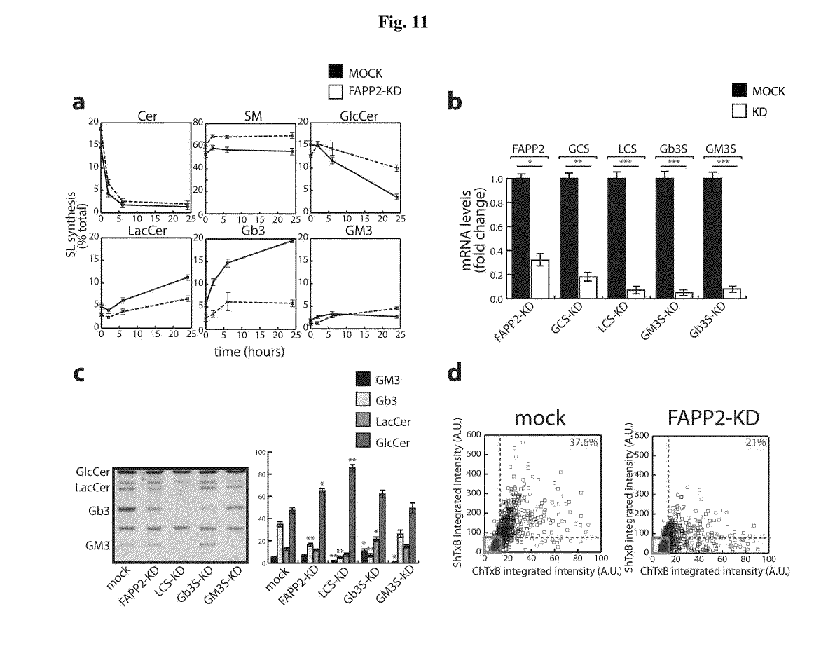

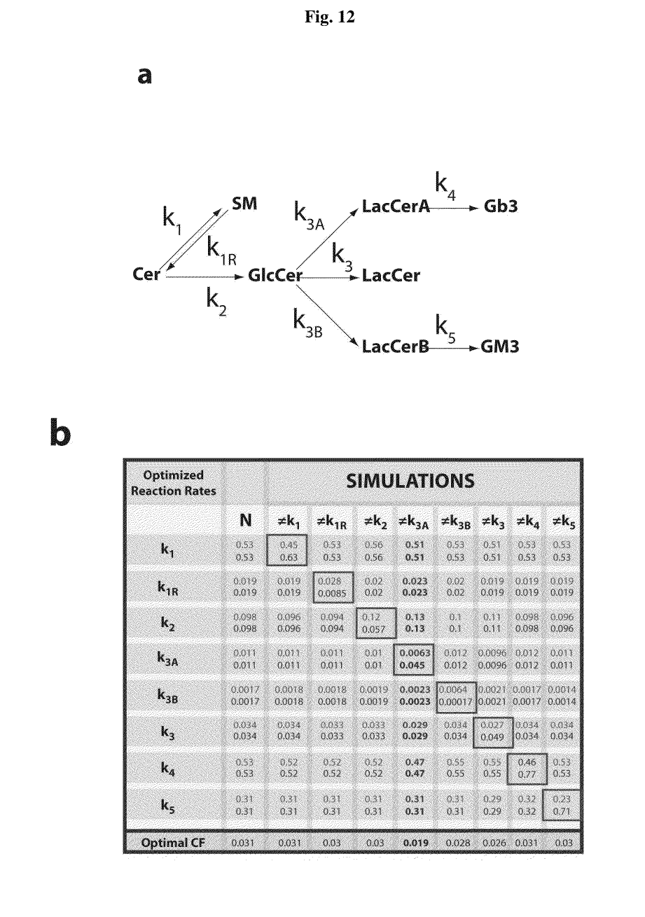

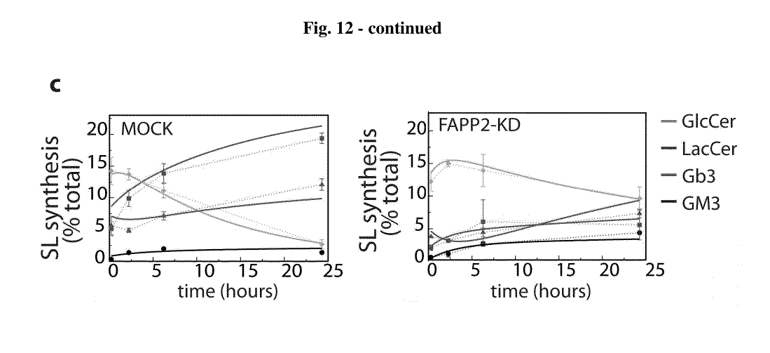

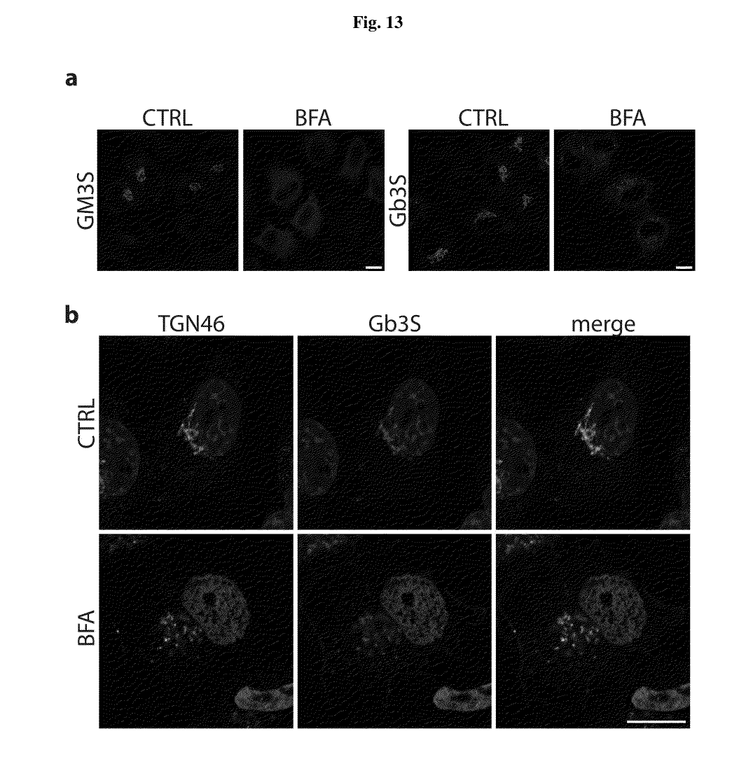

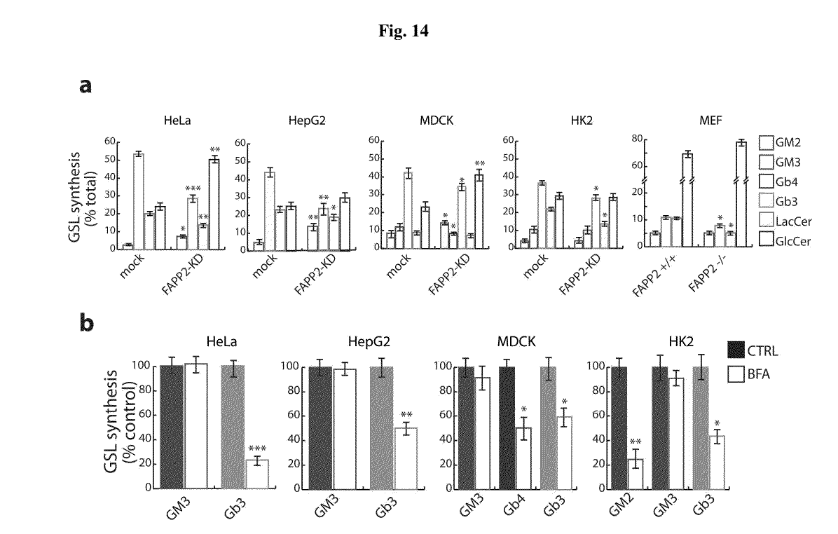

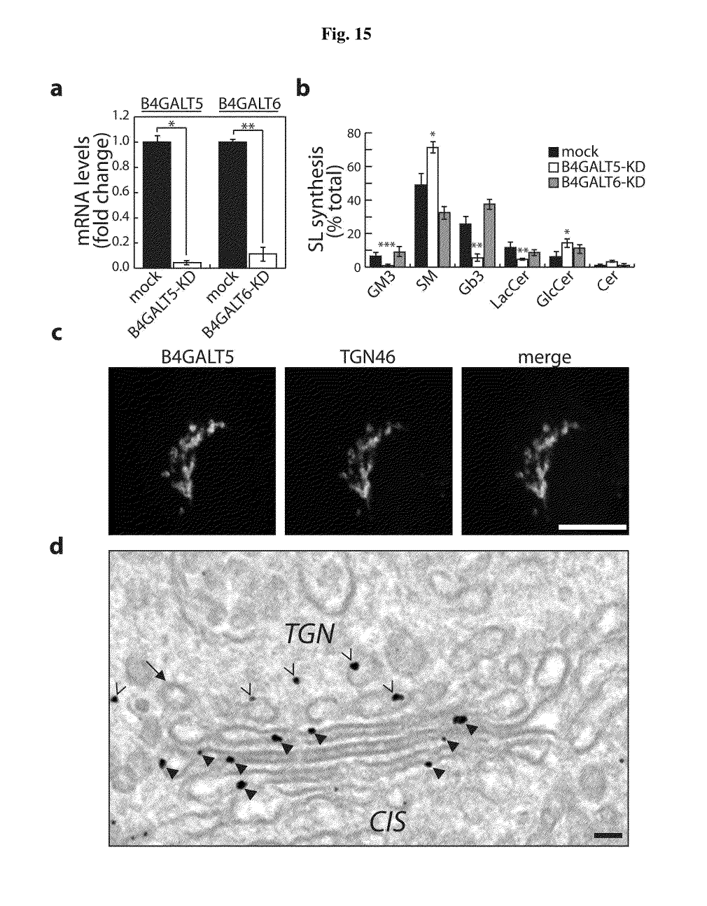

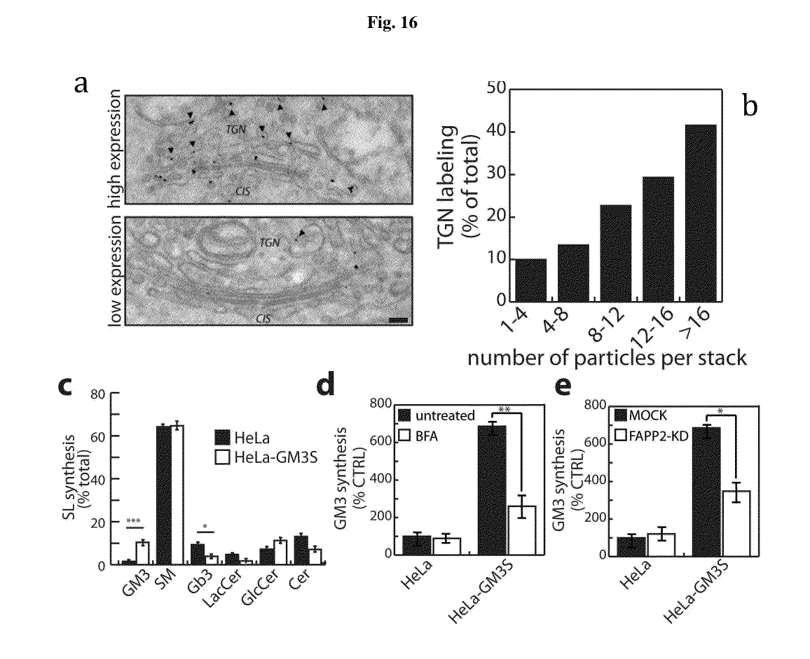

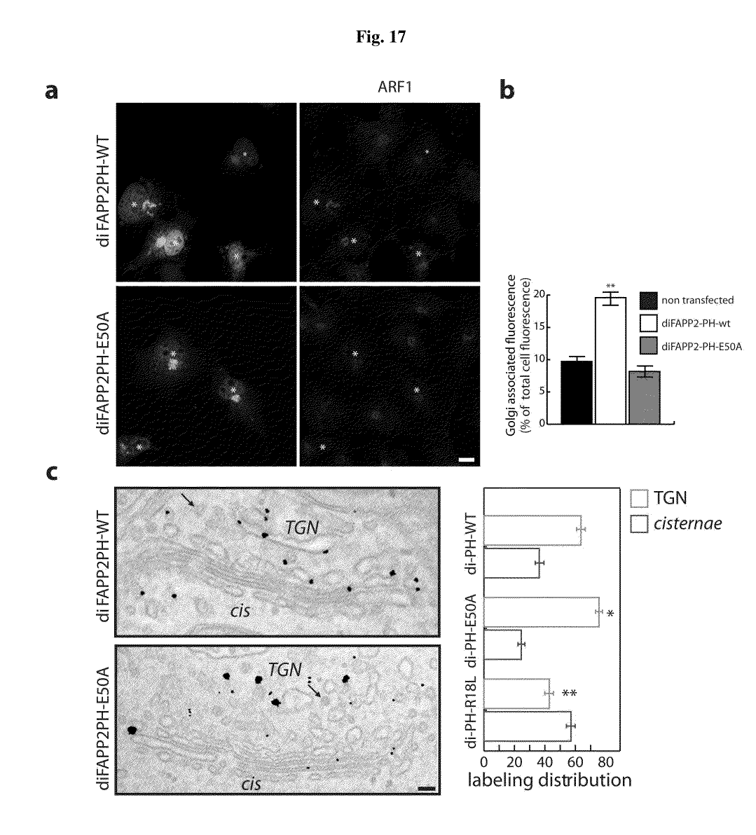

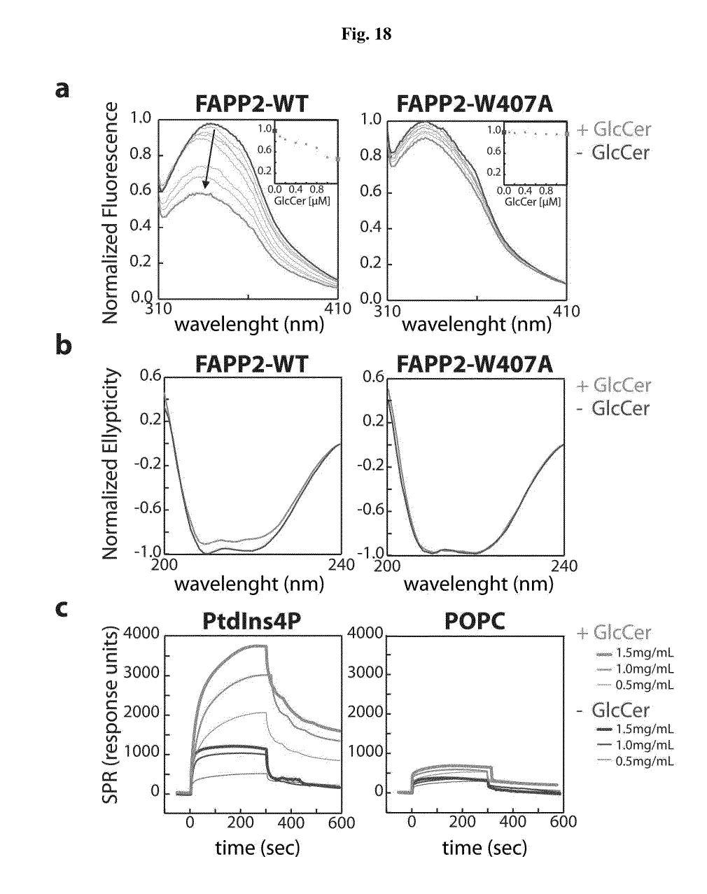

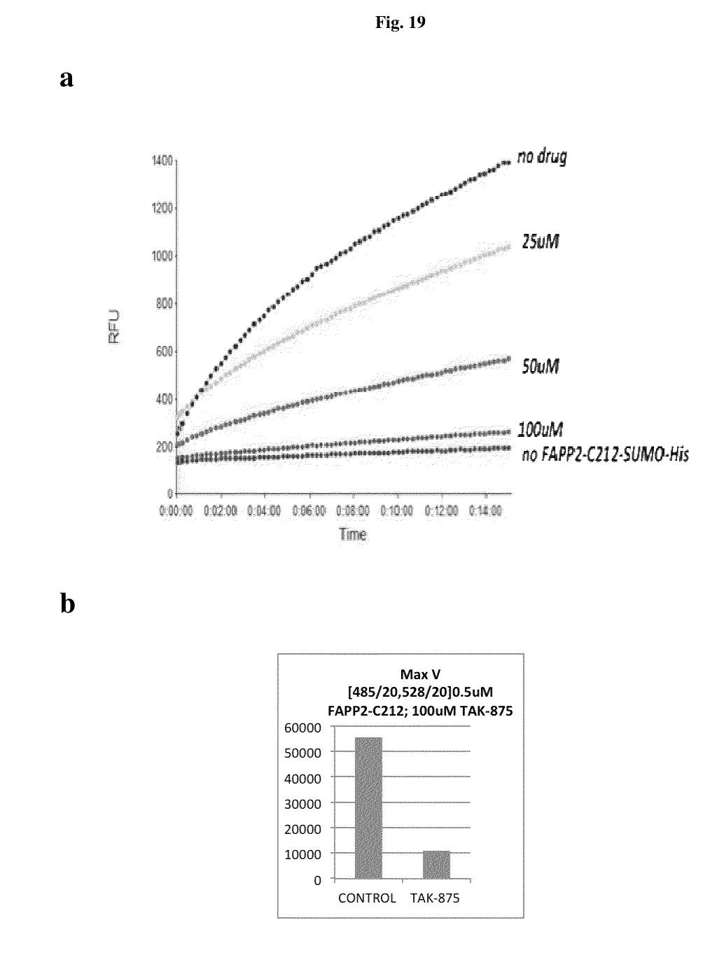

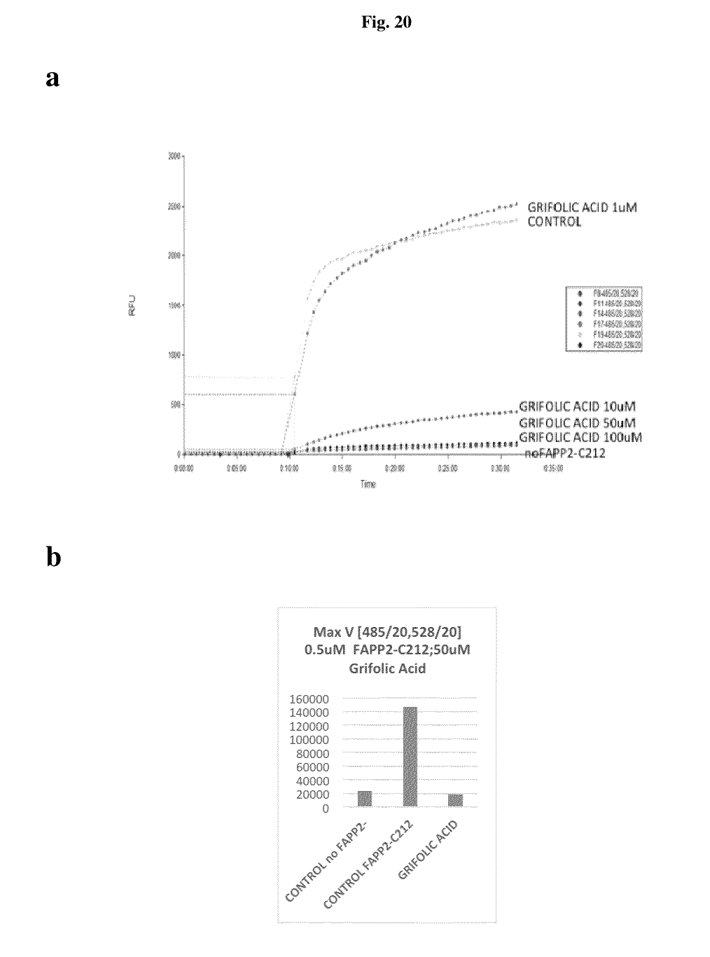

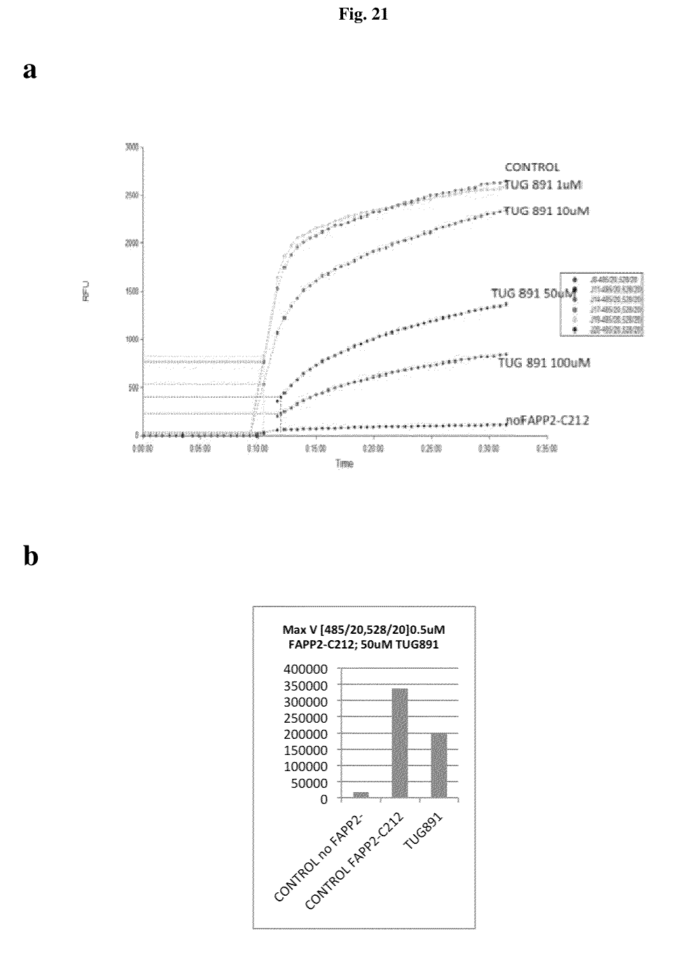

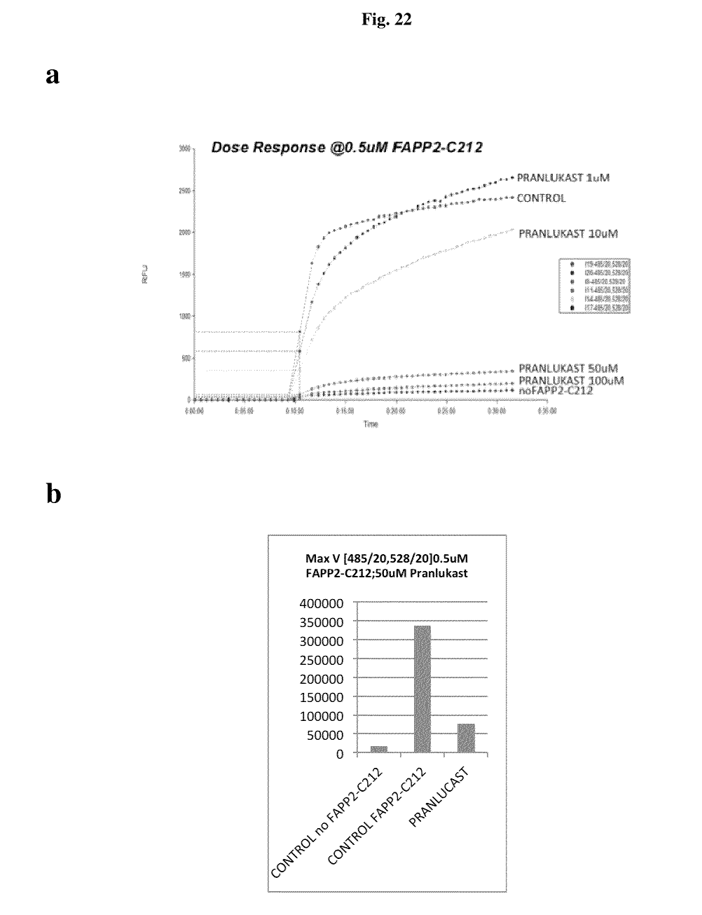

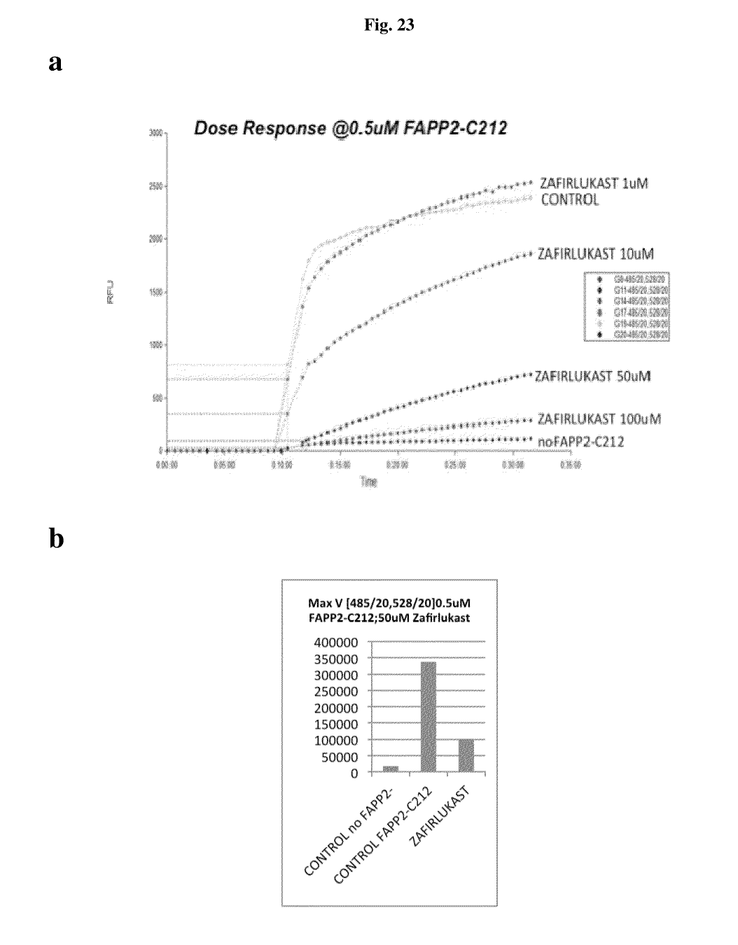

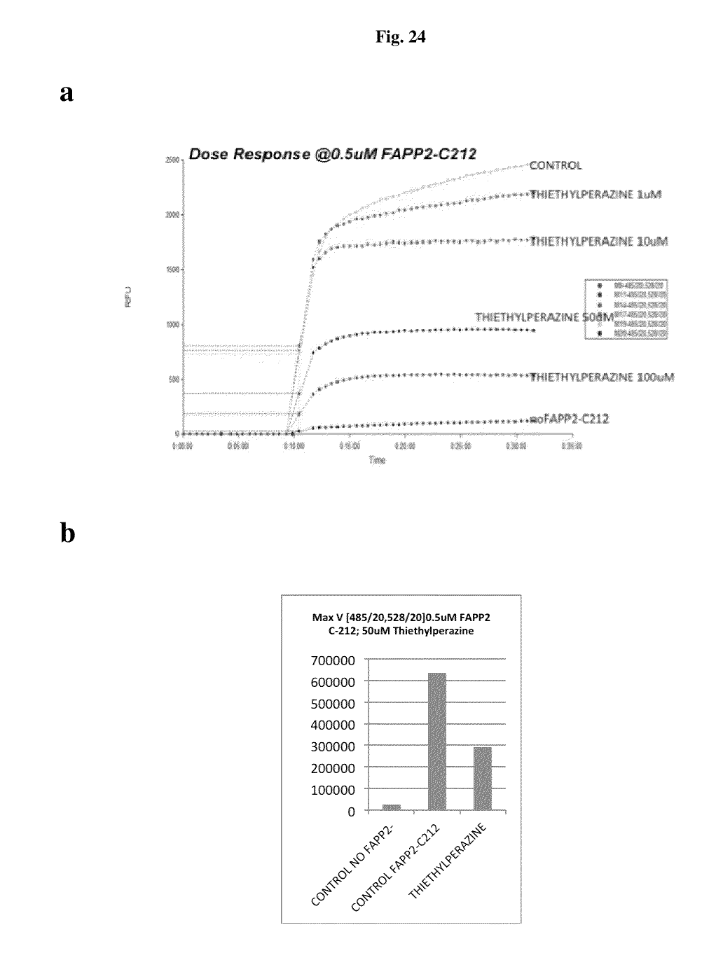

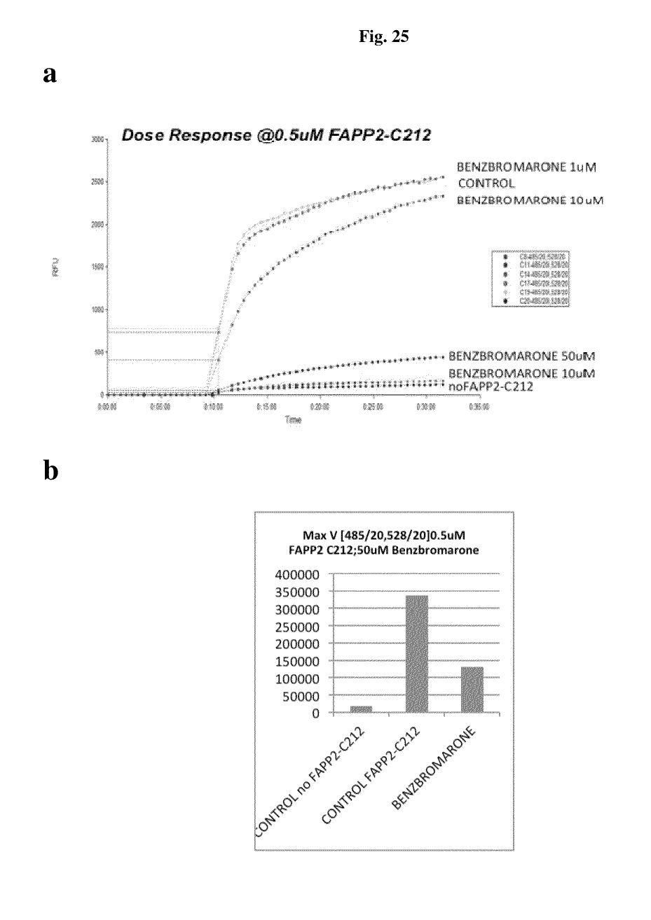

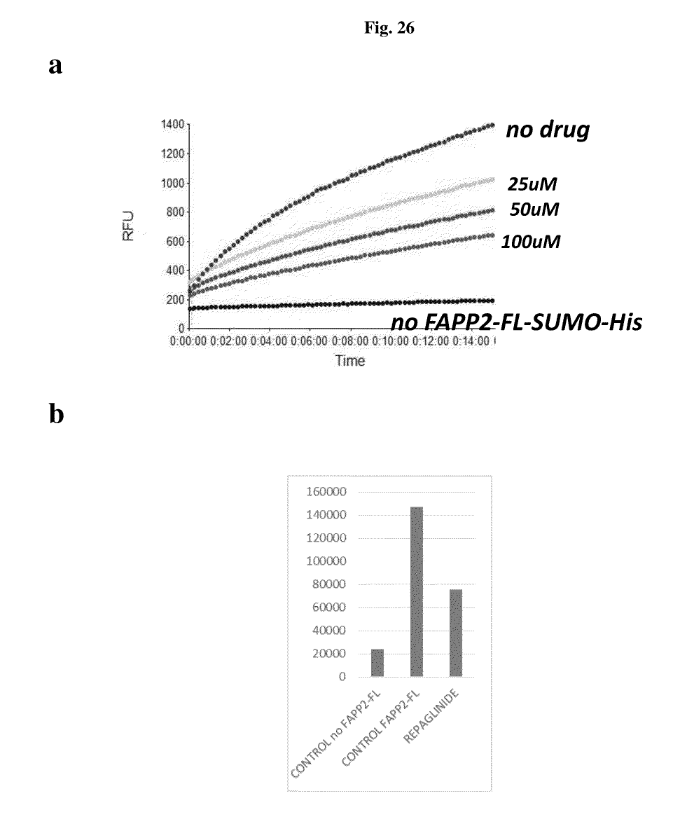

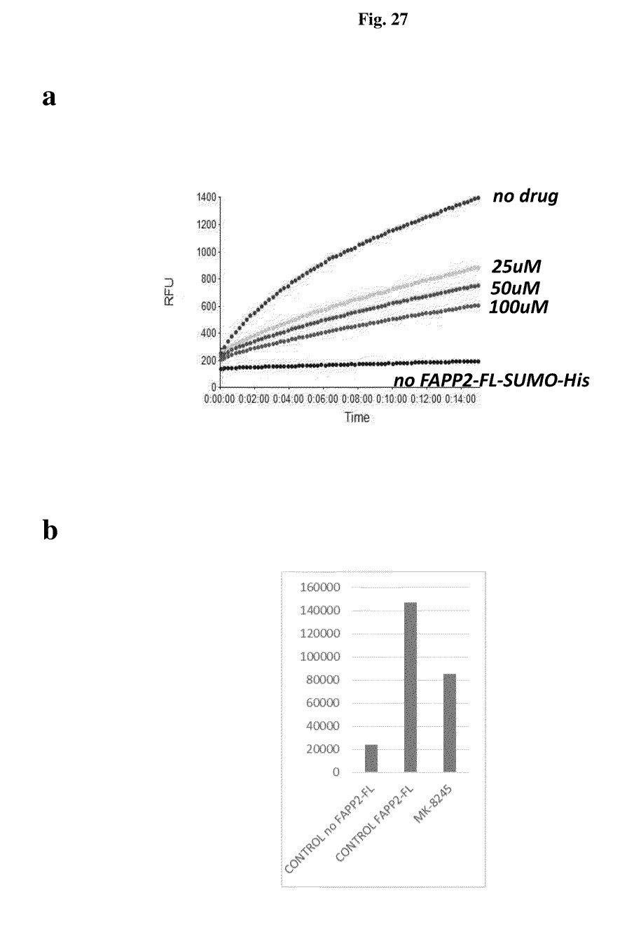

Without wishing to be bound by any particular theory, the present invention proposes that inhibiting FAPP2 might reduce the accumulation of Gb3 in cells from Fabry patients. To test this, expression of FAPP2 was reduced through siRNA and the levels/distribution of Gb3 was evaluated in fibroblast from Fabry patients using Cy3-shiga toxin (FIG. 5). Fibroblasts from six different FD patients (mutations specified above in Table 5) were left untreated or treated with siRNA specific for FAPP2 for 72 hrs and then processed for immunofluorescence and stained for Gb3 with Cy3-Shiga toxin fragment b (red) and for a lysososmal marker (LAMP1, green). Fibroblasts from Fabry patients showed an increase in total Gb3 levels (as evaluated by Shiga toxin staining) with a clear accumulation of Gb3 in lysosomes (as evaluated by LAMP1 staining). Depletion of FAPP2 induced a notable decrease of the staining.

FAPP2 was additionally validated as a target for Fabry disease (FD) patents by knocking down the expression of α-galactosidase A in HeLa cells through siRNAs and shRNAs (e.g., a cell based model for FD). FIG. 6 illustrates an exemplary result where FAPP2 KD decreases Gb3 accumulation in HeLa cells depleted of α-galoctidase A. The α-galactosidase A KD induced an accumulation of Gb3 in intracellular compartments, and this increase was counteracted by the simultaneous depletion of FAPP2 (FIG. 6). These data confirm inhibition of FAPP2 decreases Gb3 accumulation in cell models of Fabry disease.

Exemplary siRNAs and shRNAs to Human α-Galactosidase A

| Oligo # | Sequence (5′-3′) | SEQ ID No. |
|---|---|---|
| siRNA1 | GCUAUCAUGGCUGCUCCUU[dT][dT] | 90 |
| siRNA2 | GCAAUCACUGGCGAAAUUU[dT][dT] | 91 |
| siRNA3 | CAGCUUAGACAGGGAGACA[dT][dT] | 92 |
| shRNA1 | CCTGAATAGGACTGGCAGAAGCATTGTGT | 93 |
| shRNA2 | GAAGAGCCAGATTCCTGCATCAGTGAGAA | 94 |
| shRNA3 | GCAGGAGATTGGTGGACCTCGCTCTTATA | 95 |
| shRNA4 | GCTGGAATCAGCAAGTAACTCAGATGGCC | 96 |

To screen and identify small molecules for drug development for Fabry disease treatment, a miniaturized FRET-based in vitro screening assay was developed to screen for small molecules that can inhibit FAPP2. The miniaturized FRET-based in vitro screening assay tests the ability of FAPP2 to transfer GlcCer from donor to acceptor and identifies small molecules able to inhibit the transfer activity of FAPP2. The flFAPP2 was produced as Sumo-fusion protein and assayed in Rosetta cells.

FAPP2 transfers fluorescent C11-GlcCer from donor to acceptor liposomes in a concentration-dependent fashion (FIG. 7a). The transfer activity is very specific for GlcCer as short chain unlabelled GlcCer, but not ceramide, was able to inhibit transfer by competition for GlcCer transfer activity of FAPP2 (FIG. 7b). The final concentration of both C8-GlcCer and C6-Cer was 10 uM.

Without wishing to be bound by any particular theory, the present invention proposes glucoside phlorizin, which inhibits glucose reabsorption acting on SGLT transporters, also act as FAPP2 inhibitors. This is supported by homology modelling of GLTP-domain of FAPP2 on the GLTP itself and hypothesizing that the interaction of GlcCer with FAPP2 could be mainly mediated by the Glucosyl moiety of GlcCer. Phlorizin was tested at different concentrations (100 uM, 200 uM, 5000 uM, 1 mM) and found that the drug can inhibit the GlcCer transfer activity of FAPP2 (FIG. 7c). Surprisingly, dapaglifozin, a derivative of phlorizin had no inhibitory activity (FIG. 7d).

### Example 9

### Further FAPP2 Inhibitors

Starting from the promising results using the antidiabetic Phlorizin, the ability of other anti-diabetic compounds to inhibit FAPP2-mediated GlcCer transfer was tested. The Selleckchem Anti-Diabetic Library containing 31 active compounds was tested using an in vitro transfer assay and TAK-875 was identified as a FAPP2 inhibitor.

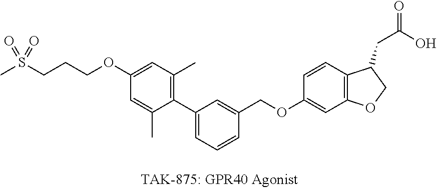

TAK-875 (Fasiglifam) is a selective agonist of GPR40 (Free fatty acid receptor 1), a G-protein coupled receptor in the islet cells of the pancreas. The activation of GPR40 improves glucose-dependent insulin secretion from the beta cells with minimal hypoglycemia and an improved HbA1c (Glycated hemoglobin).

TAK-875 activity was assessed using Fluorescence resonance energy transfer assay (FIG. 19). The assay was conducted at three different drug concentrations (100 uM, 50 uM, 25 uM) and FAPP2 at 0.5 uM. Inhibition of FAPP2 activity by TAK-875 was measured for 15 mins 100 uM TAK-875 significantly reduced FAPP2-mediated GlcCer transfer (FIG. 19A). FIG. 19B shows the effect of 100 uM TAK-875 on FAPP2 velocity transfer at time zero.

The identification of TAK-875 prompted the inventors to test other GPR agonists. Grifolic Acid was the most active FAPP2 inhibitor in vitro.

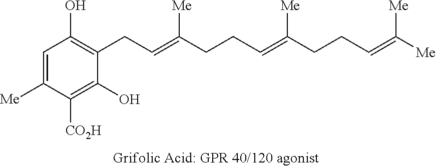 [SMILES: CC(C)=CCC/C(C)=C/CC/C(C)=C/Cc1c(O)cc(C)c(C(=O)O)c1O]

Grifolic Acid is a novel and selective agonist for the Free Fatty Acid Receptors (FFARs) GPR 120 and GPR 40. GPR120 and GPR40 are G-protein-coupled receptors whose endogenous ligands are medium- and long-chain free fatty acids, and they are thought to play an important physiological role in insulin release.

Grifolic Acid activity was assessed using Fluorescence resonance energy transfer assay (FIG. 20). The assay was conducted at four different drug concentrations (100 uM, 50 uM, 10 uM, 1 uM) and FAPP2-C212 at 0.5 uM Inhibition of FAPP2 activity by Grifolic Acid was measured for 30 mins 100 uM and 50 uM Grifolic Acid significantly reduced FAPP2-mediated GlcCer transfer (FIG. 20A). FIG. 20B shows the effect of 50 uM of Grifolic Acid on FAPP2 velocity transfer at time zero.

TUG-891 also belongs to the GPR agonist super family and like TAK-875 and Grifolic Acid is able to modulate FAPP2 transfer activity in vitro. TUG-891 was recently described as a potent and selective agonist for the long chain free fatty acid (LCFA) receptor 4 (FFA4;or GPR120).

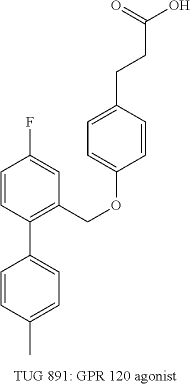 [SMILES: Cc1ccc(-c2ccc(F)cc2COc2ccc(CCC(=O)O)cc2)cc1]

TUG 891 activity was assessed using Fluorescence resonance energy transfer assay (FIG. 21). The assay was conducted at four different drug concentrations (100 uM, 50 uM, 10 uM, 1 uM) and FAPP2-C212 at 0.5 uM Inhibition of FAPP2 activity by TUG 891 was measured for 30 mins 50 uM TUG-891 inhibits 50% FAPP2 transfer activity (FIG. 21A). FIG. 21B shows the effect of 50 uM TUG-891 on FAPP2 velocity transfer at time zero.

Pursuing the identification of a new class of drugs able to inhibit FAPP2 activity, the inventors screen a wider and heterogeneous library of pharmaceutically active compounds. The selected library was the Prestwick Chemical Library® containing 1280 small molecules, 100% approved drugs (FDA, EMEA and other agencies).

One of the identified drugs was Pranlukast, a cysteinyl leukotriene receptor 1 antagonist used for the maintenance treatment of asthma. Interestingly, Cysteinyl leukotrienes (CysLTs) are a family of inflammatory lipid mediators synthesized from arachidonic acid and this may explain why Pranlukast is able to inhibit FAPP2 binding to GlcCer

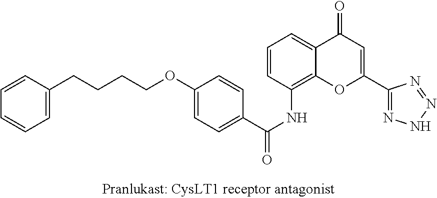 [SMILES: O=C(Nc1cccc2c(=O)cc(-c3nn[nH]n3)oc12)c1ccc(OCCCCc2ccccc2)cc1]

Pranlukast activity was assessed using Fluorescence resonance energy transfer assay (FIG. 22). The assay was conducted at four different drug concentrations (100 uM, 50 uM, 10 uM, 1 uM) and FAPP2-C212 at 0.5 uM Inhibition of FAPP2 activity by Pranlukast was measured for 30 mins 50 uM Pranlukast inhibits 90% FAPP2 transfer activity (FIG. 22A). FIG. 22B shows the effect of 50 uM Pranlukast on FAPP2 velocity transfer at time zero.

Zafirlukast is an oral leukotriene receptor antagonist LTRA for the maintenance treatment of asthma. Zafirlukast has been identified as a FAPP2 inhibitor by screening the Prestwick Chemical Library®.

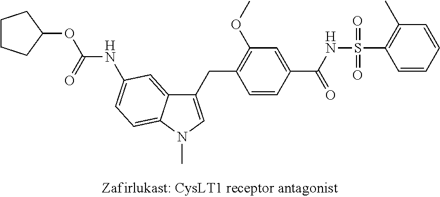 [SMILES: COc1cc(C(=O)NS(=O)(=O)c2ccccc2C)ccc1Cc1cn(C)c2ccc(NC(=O)OC3CCCC3)cc12]

Zafirlukast activity was assessed using Fluorescence resonance energy transfer assay (FIG. 23). The assay was conducted at four different drug concentrations (100 uM, 50 uM, 10 uM, 1 uM) and FAPP2-C212 at 0.5 uM Inhibition of FAPP2 activity by Zafirlukast was measured for 30 mins 50 uM Zafirlukast inhibits 90% FAPP2 transfer activity (FIG. 23A). FIG. 23B shows the effect of 50 uM Zafirlukast on FAPP2 velocity transfer at time zero.

During the Prestwick Chemical Library® screening, a different class of compounds was found to have inhibitory effects on FAPP2 transport activity, the phenothiazine.

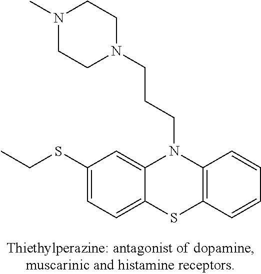 [SMILES: CCSc1ccc2c(c1)N(CCCN1CCN(C)CC1)c1ccccc1S2]

Thiethylperazine (Torecan) is an antiemetic of the phenothiazine class. Though it was never licensed or used as an antipsychotic, it may have such effects. Thiethylperazine is an antagonist at types 1, 2, and 4 dopamine receptors, 5-HT receptor types 2A and 2C, muscarinic receptors 1 through 5, alpha(1)-receptors, and histamine H1-receptors. Thiethylperazine's antipsychotic effect is due to antagonism at dopamine and serotonin type 2 receptors, with greater activity at serotonin 5-HT2 receptors than at dopamine type-2 receptors.

Thiethylperazine activity was assessed using Fluorescence resonance energy transfer assay (FIG. 24). The assay was conducted at four different drug concentrations (100 uM, 50 uM, 10 uM, 1 uM) and FAPP2-C212 at 0.5 uM Inhibition of FAPP2 activity by Thiethylperazine was measured for 30 mins. 50 uM Thiethylperazine inhibits 60% FAPP2 transfer activity (FIG. 24A). FIG. 24B shows the effect of 50 uM Thiethylperazine on FAPP2 velocity transfer at time zero.

Benzbromarone was identified as a FAPP2 inhibitor by screening the Prestwick Chemical Library®, and up to now represents one of the most potent FAPP2 inhibitors that has been identified in vitro.

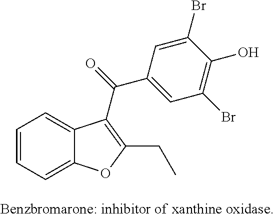 [SMILES: CCc1oc2ccccc2c1C(=O)c1cc(Br)c(O)c(Br)c1]

Benzbromarone is a uricosuric agent and non-competitive inhibitor of xanthine oxidase used in the treatment of gout, especially when allopurinol, a first-line treatment, fails or produces intolerable adverse effects. It is structurally related to the antiarrhythmic amiodarone.

Benzbromarone activity was assessed using Fluorescence resonance energy transfer assay (FIG. 25). The assay was conducted at four different drug concentrations (100 uM, 50 uM, 10 uM, 1 uM) and FAPP2-C212 at 0.5 uM Inhibition of FAPP2 activity by Benzbromarone was measured for 30 mins. 50 uM Benzbromarone inhibits 80% FAPP2 transfer activity (FIG. 25A). FIG. 25B shows the effect of 50 uM Benzbromarone on FAPP2 velocity transfer at time zero.

Repaglinide is an oral antihyperglycemic agent used for the treatment of non-insulin-dependent diabetes mellitus (NIDDM). Like the sulphonylureas, repaglinide acts by stimulating release of insulin from the β cells of the islets of pancreas inhibiting ATP-sensitive K+ channels, thereby activating the Ca++ channels with increase in intracellular calcium to release insulin.

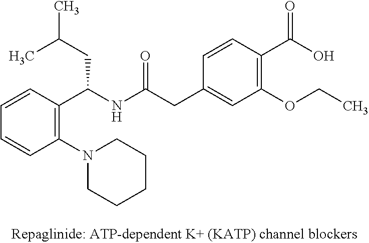 [SMILES: CCOc1cc(CC(=O)N[C@@H](CC(C)C)c2ccccc2N2CCCCC2)ccc1C(=O)O]

Repaglinide activity was assessed using Fluorescence resonance energy transfer assay (FIG. 26). The assay was conducted at three different drug concentrations (100 uM, 50 uM, 25 uM) and FAPP2-FL-SUMO-His at 0.5 uM Inhibition of FAPP2 activity by Repaglinide was measured for 15 mins. 50 uM Repaglinide inhibits 50% FAPP2 transfer activity (FIG. 26A). FIG. 26B shows the inhibition rate of 50 uM Repaglinide on FAPP2-FL velocity transfer at time zero.

MK-8245 is a stearoyl-CoA desaturase (SCD) inhibitor with preclinical antidiabetic and antidyslipidemic efficacy with a significantly improved therapeutic window. MK-8245 is currently being developed by MercK.

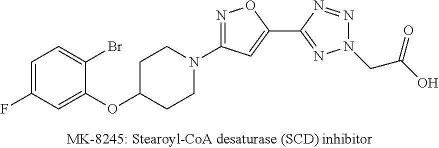 [SMILES: O=C(O)Cn1nnc(-c2cc(N3CCC(Oc4cc(F)ccc4Br)CC3)no2)n1]

MK-8245 activity was assessed using Fluorescence resonance energy transfer assay (FIG. 27). The assay was conducted at three different drug concentrations (100 uM, 50 uM, 25 uM) and FAPP2-FL-SUMO-His at 0.5 uM Inhibition of FAPP2 activity by MK-8245 was measured for 15 mins. 50 uM MK-8245 inhibits 40% FAPP2 transfer activity (FIG. 27A). FIG. 27B shows the inhibition rate of 50 uM MK-8245 on FAPP2-FL velocity transfer at time zero

### Example 10

### FAPP2 Inhibitors Chemical Synthesis

C-aryl glucoside and O-aryl glucoside of the present invention may be synthesed according to methods known to the skilled person in the art. In particular such methods may be found in Martin S. Pure Appl. Chem., Vol. 75, No. 1, pp. 63-70, 2003 and in Synthesis and characterization of Glycosides, Springer, chap. 2, O-Glycoside Formation, Brito-Arias, M. 2007, XII, 351 p., Hardcover (ISBN: 978-0-387-26251-2).

### Example 11

### Analysis of the Biological Effects of FAPP2 Inhibitors on Gb3 Levels in Fabry Disease Cell Model

The present Example demonstrates that FAPP2 inhibition decreases Gb3 levels in a Fabry disease cell model (FIG. 28). Hela-shGLA (clone 4G) cells (i.e. the above described HeLa cells stably knocked down for GLA using the above described shRNA) were plated in 384-well plates (500 cells/well). 24 hours later cells were treated with ten different FAPP2 transfer activity inhibitors. Compounds were diluted in complete culture medium at concentrations of 10 μM and 50 μM, and cells were incubated at 37° C. for 72 hours. Four replicates for each condition were tested. Control cells were treated with 0.1% DMSO (negative control) or 10 μM PDMP (positive control). After incubation, cells were fixed in 4% PFA and stained with Cy3-Shiga toxin B, anti-LampI antibody, and Hoechst 33342. Images were captured using the Operetta System; the intensity of Gb3 spots (detected by Shiga toxin B) in lysosomes (detected by LampI) were calculated for each condition. Five fields per well were analyzed (about 1000 cells/well in control wells).

Ten of the compounds found to be active in inhibiting the GlcCer transfer activity on FAPP2 were tested to assess whether they had a biological effect in a Fabry disease cell model in lowering the levels of Gb3, as highlighted through the staining with Cy3/Shiga toxin. This effect has been clearly confirmed in comparison to the negative control (DMSO treated cells). As a positive control we have used PDMP (D-threo-lphenyl-2-decanoylamino-3-morpholino-1-propanol), a general inhibitor of protein and lipid glycosylation that cannot be used in clinics because of its toxicity).

### REFERENCES

1 D'Angelo, G. et al. Glycosphingolipid synthesis requires FAPP2 transfer of glucosylceramide Nature 449, 62-67, (2007).
2 Hakomori, S. I. Structure and function of glycosphingolipids and sphingolipids: recollections and future trends. Biochim Biophys Acta 1780, 325-346, (2008).
3 Jeckel, D., Karrenbauer, A., Burger, K. N., van Meer, G. & Wieland, F. Glucosylceramide is synthesized at the cytosolic surface of various Golgi subfractions. J Cell Biol 117, 259-267, (1992).
4 Futerman, A. H. & Pagano, R. E. Determination of the intracellular sites and topology of glucosylceramide synthesis in rat liver. Biochem J 280 (Pt 2), 295-302, (1991).
5 Hannun, Y. A. & Obeid, L. M. Principles of bioactive lipid signalling: lessons from sphingolipids. Nat Rev Mol Cell Biol 9, 139-150, (2008).
6 Halter, D. et al. Pre- and post-Golgi translocation of glucosylceramide in glycosphingolipid synthesis. J Cell Biol 179, 101-115, (2007).
7 Maccioni, H. J., Quiroga, R. & Ferrari, M. L. Cellular and molecular biology of glycosphingolipid glycosylation. J Neurochem 117, 589-602, (2011).
8 Jacewicz, M., Clausen, H., Nudelman, E., Donohue-Rolfe, A. & Keusch, G. T. Pathogenesis of shigella diarrhea. XI. Isolation of a shigella toxin-binding glycolipid from rabbit jejunum and HeLa cells and its identification as globotriaosylceramide. J Exp Med 163, 1391-1404, (1986).
9 Heyningen, S. V. Cholera toxin: interaction of subunits with ganglioside GM1. Science 183, 656-657, (1974).
10 Psotka, M. A. et al. Shiga toxin 2 targets the murine renal collecting duct epithelium. Infect Immun 77, 959-969, (2009).
11 Okuda, T. et al. Targeted disruption of Gb3/CD77 synthase gene resulted in the complete deletion of globo-series glycosphingolipids and loss of sensitivity to verotoxins. J Biol Chem 281, 10230-10235, (2006).
12 D'Angelo, G., Vicinanza, M. & De Matteis, M. A. Lipid-transfer proteins in biosynthetic pathways. Curr Opin Cell Biol 20, 360-370, (2008).
13 Chege, N. W. & Pfeffer, S. R. Compartmentation of the Golgi complex: brefeldin-A distinguishes trans-Golgi cisternae from the trans-Golgi network. J Cell Biol 111, 893-899, (1990).
14 Godi, A. et al. FAPPs control Golgi-to-cell-surface membrane traffic by binding to ARF and PtdIns(4)P. Nat Cell Biol 6, 393-404, (2004).
15 Yamaji, T., Nishikawa, K. & Hanada, K. Transmembrane BAX inhibitor motif containing (TMBIM) family proteins perturbs a trans-Golgi network enzyme, Gb3 synthase, and reduces Gb3 biosynthesis. J Biol Chem 285, 35505-35518, (2010).
16 San Pietro, E. et al. Group IV phospholipase A(2)alpha controls the formation of inter-cisternal continuities involved in intra-Golgi transport. PLoS Biol 7, e1000194, (2009).
17 Ichikawa, S., Nakajo, N., Sakiyama, H. & Hirabayashi, Y. A mouse B16 melanoma mutant deficient in glycolipids. Proc Natl Acad Sci USA 91, 2703-2707, (1994).
18 He, J. et al. Molecular basis of phosphatidylinositol 4-phosphate and ARF1 GTPase recognition by the FAPP1 pleckstrin homology (PH) domain. J Biol Chem 286, 18650-18657, (2011).
19 D'Angelo, G., Vicinanza, M., Di Campli, A. & De Matteis, M. A. The multiple roles of PtdIns(4)P—not just the precursor of PtdIns(4,5)P2. J Cell Sci 121, 1955-1963, (2008).
20 Mallard, F. & Johannes, L. Shiga toxin B-subunit as a tool to study retrograde transport. Methods Mol Med 73, 209-220, (2003)
21 D'Angelo, G. et al. GRASP65 and GRASP55 sequentially promote the transport of C-terminal valine-bearing cargos to and through the Golgi complex. J Biol Chem 284, 34849-34860, (2009).
22 Zhai, X. et al. Glycolipid acquisition by human glycolipid transfer protein dramatically alters intrinsic tryptophan fluorescence: insights into glycolipid binding affinity. J Biol Chem 284, 13620-13628, (2009).
23 Ohvo-Rekila, H. & Mattjus, P. Monitoring glycolipid transfer protein activity and membrane interaction with the surface plasmon resonance technique. Biochim Biophys Acta 1808, 47-54, (2011).
24 Sato, T. et al. The Rabb GTPase regulates apical protein localization in intestinal cells. Nature 448, 366-369, (2007).
25 Kamlekar, R. K. et al. The glycolipid transfer protein (GLTP) domain of phosphoinositol 4-phosphate adaptor protein-2 (FAPP2): Structure drives preference for simple neutral glycosphingolipids. Biochim Biophys Acta 1831, 417-427, (2013).

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition. Where the claims recite a composition, it should be understood that the invention encompasses methods of using the composition and methods of making the composition.

### INCORPORATION OF REFERENCES

All publications and patent documents cited in this application are incorporated by reference in their entirety to the same extent as if the contents of each individual publication or patent document were incorporated herein.

1. (canceled)

2. (canceled)

3. A method of reducing globotrioaosylceramide (Gb3) accumulation in a cell, comprising administering to a cell having or susceptible to Gb3 accumulation an effective amount of an inhibitor of phosphatidylinositol-4-phosphate adaptor-2 (FAPP2).

4. A method of treating a disease, disorder or condition associated with globotrioaosylceramide (Gb3) accumulation, comprising administering to a subject in need of treatment an effective amount of an inhibitor of phosphatidylinositol-4-phosphate adaptor-2 (FAPP2).

5. The method of claim 3, wherein the cell is a mammalian cell.

6. The method of claim 3, wherein the cell is a cultured cell.

7. The method of claim 3, wherein the cell is a cell of an organism.

8. The method of claim 4, wherein said inhibitor is an aryl glucoside compound comprising a glycosidic linkage, or an interfering oligonucleotide.

9. The method of claim 8, wherein said aryl glucoside compound is a C-aryl glucoside compound or an O-aryl glucoside compound.

10. The method of claim 8, wherein the aryl glucoside compound has a structure of formula I: 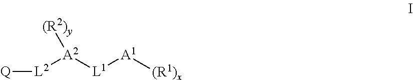
or a pharmaceutically acceptable salt thereof, wherein:
Q is a monosaccharide or modified monosaccharide;
A¹ is phenyl or a 5-6 membered heteroaryl ring having 1-3 heteroatoms independently selected from nitrogen, oxygen and sulfur;
A² is phenyl or a 5-6 membered heteroaryl ring having 1-3 heteroatoms independently selected from nitrogen, oxygen and sulfur;
L¹ is a covalent bond, or a C₁₋₄ bivalent straight or branched hydrocarbon chain, wherein one or two methylene units of the chain are optionally and independently replaced by —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
L² is a covalent bond or —O—;
each R¹ is independently halogen, —CN, —R; —OR; —SR; —N(R)₂; —N(R)C(O)R; —C(O)N(R)₂; —N(R)C(O)N(R)₂; —N(R)C(O)OR; —OC(O)N(R)₂; —N(R)S(O)₂R; —S(O)₂N(R)₂; —OC(O)OR; —C(O)R; —OC(O)R; —C(O)OR; —S(O)R; —S(O)₂R; or Cy;
each R² is independently halogen, —CN, —R, —OR, —SR, —N(R)₂, —N(R)C(O)R, —C(O)N(R)₂, —N(R)C(O)N(R)₂, —N(R)C(O)OR, —OC(O)N(R)₂, —N(R)SO₂R, —S(O)₂N(R)₂, —C(O)R, —C(O)OR, —OC(O)R, —S(O)R, or —S(O)₂R;
Cy is a ring, substituted with p instances of R³; wherein said ring is selected from the group consisting of a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R is independently hydrogen, deuterium, or an optionally substituted group selected from C₁₋₆ aliphatic; a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, —B(OR)₂, or an optionally substituted ring selected from phenyl and 5-6 membered heteroaryl having 1-4 heteroatoms independently selected from nitrogen, oxygen, and sulfur;
p is 1-5; x is 0-5; and y is 0-4.

11. The method of claim 8, wherein the aryl glucoside compound has a structure of formula II-a or II-b: 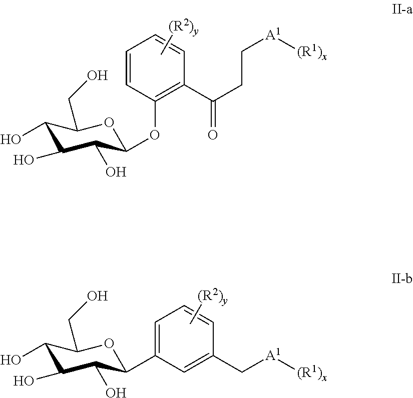
or a pharmaceutically acceptable salt thereof, wherein:
A¹ is phenyl or a 5-6 membered heteroaryl ring having 1-3 heteroatoms independently selected from nitrogen, oxygen and sulfur;
each R¹ is independently halogen, —CN, —R; —OR; —SR; —N(R)₂; —N(R)C(O)R; —C(O)N(R)₂; —N(R)C(O)N(R)₂; —N(R)C(O)OR; —OC(O)N(R)₂; —N(R)S(O)₂R; —S(O)₂N(R)₂; —OC(O)OR; —C(O)R; —OC(O)R; —C(O)OR; —S(O)R; —S(O)₂R; or Cy;
each R² is independently halogen, —CN, —R, —OR, —SR, —N(R)₂, —N(R)C(O)R, —C(O)N(R)₂, —N(R)C(O)N(R)₂, —N(R)C(O)OR, —OC(O)N(R)₂, —N(R)SO₂R, —S(O)₂N(R)₂, —C(O)R, —C(O)OR, —OC(O)R, —S(O)R, or —S(O)₂R;
Cy is a ring, substituted with p instances of R³; wherein said ring is selected from the group consisting of a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R is independently hydrogen, deuterium, or an optionally substituted group selected from C₁₋₆ aliphatic; a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, —B(OR)₂, or an optionally substituted ring selected from phenyl and 5-6 membered heteroaryl having 1-4 heteroatoms independently selected from nitrogen, oxygen, and sulfur;
p is 1-5; x is 0-5; and y is 0-4.

12. The method of claim 8, wherein the aryl glucoside compound has a structure selected from the group consisting of: 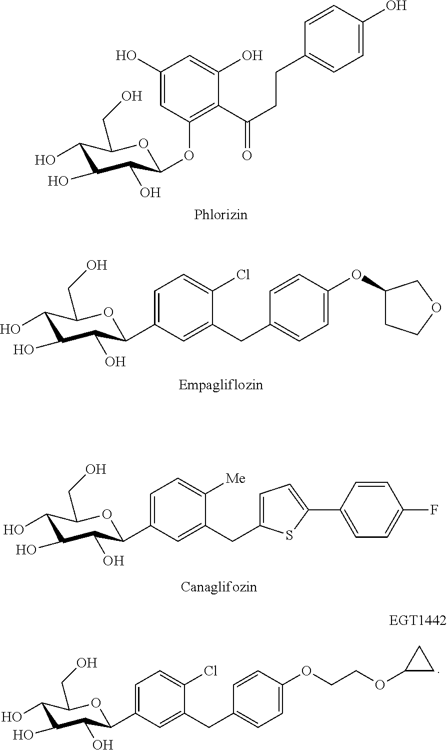
or a pharmaceutically acceptable salt thereof.

13. The method of claim 12, wherein the aryl glucoside compound has a structure of 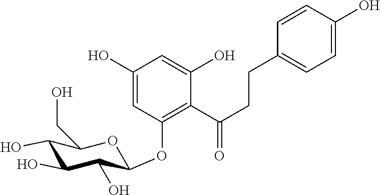
or a pharmaceutically acceptable salt thereof.

14. The method of claim 8, wherein the aryl glucoside compound has a structure selected from the group consisting of: 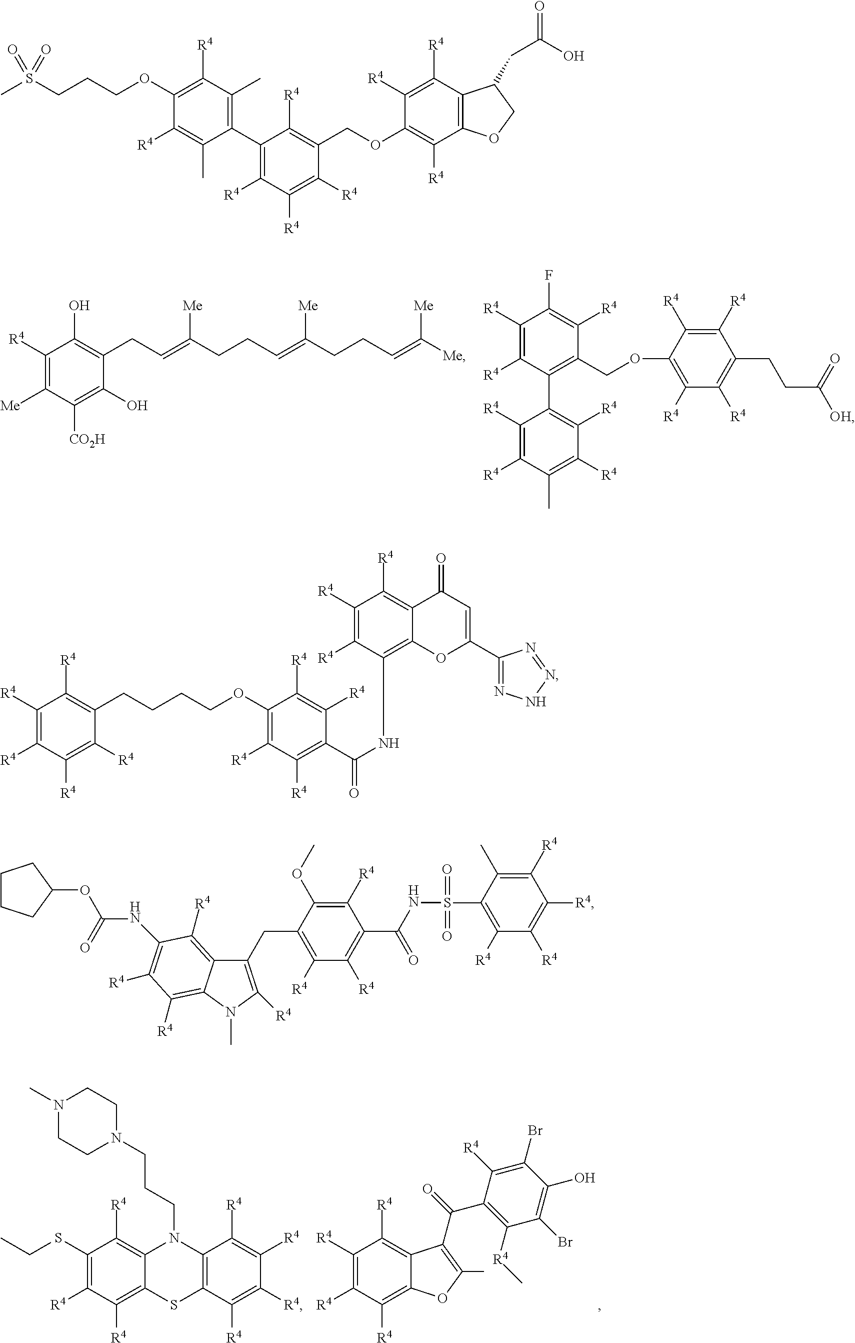 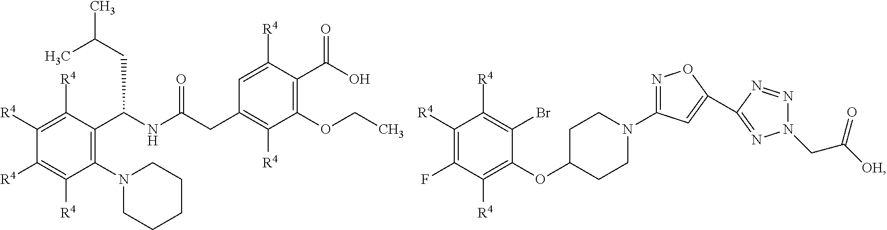
or pharmaceutically acceptable salts thereof,
wherein each R⁴ can be the same or different and is selected from the group consisting of H and -L²-Q, wherein Q is a monosaccharide or modified monosaccharide and L² is a covalent bond or —O—, provided that the aryl glucoside compound includes at least one glycosidic linkage.

15. The method of claim 14, wherein the aryl glucoside compound comprises one glycosidic linkage.

16. The inhibitor or the method of claim 4, wherein said inhibitor has a structure selected from the group consisting of: 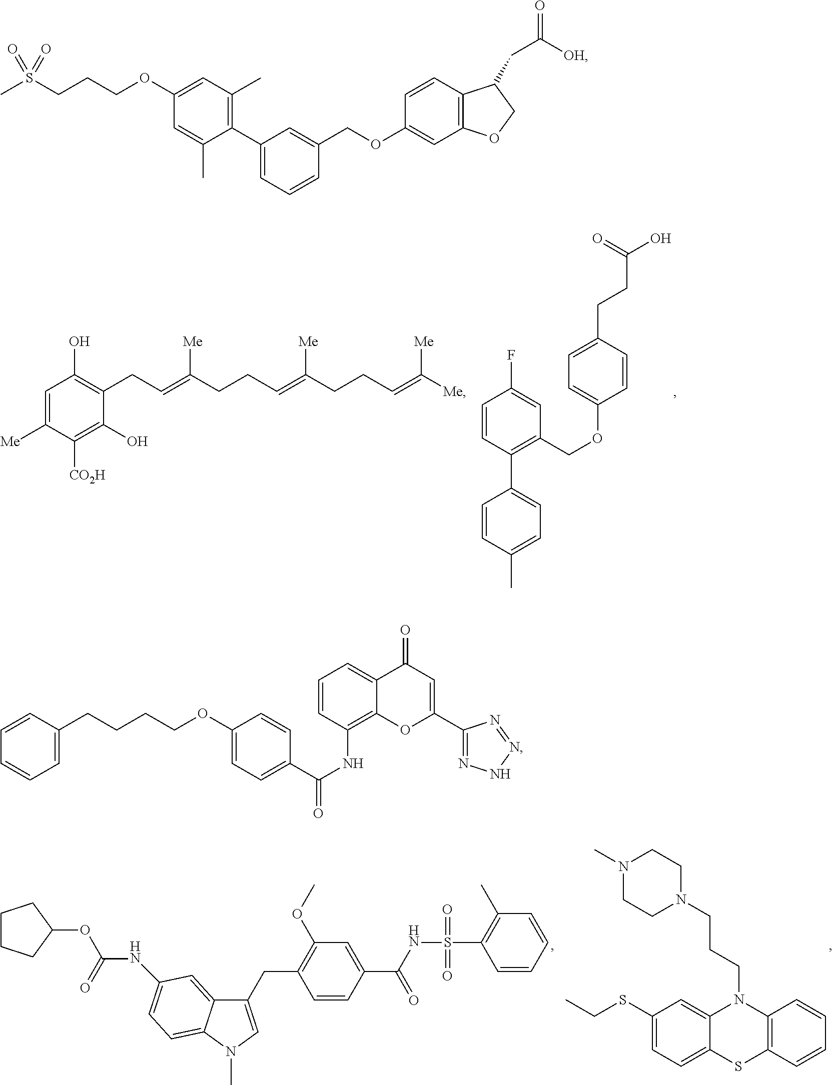 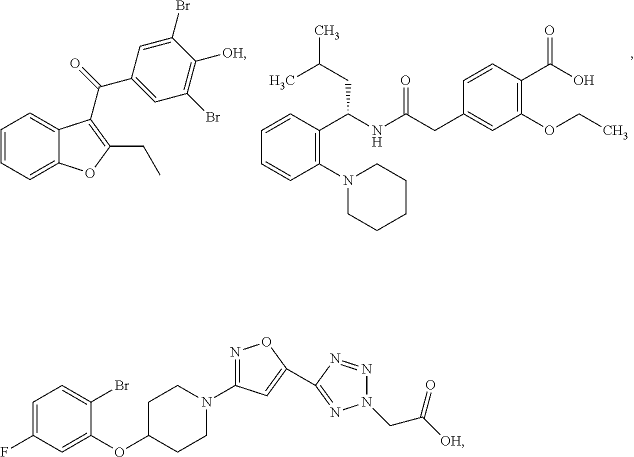 [SMILES: CCc1oc2ccccc2c1C(=O)c1cc(Br)c(O)c(Br)c1 ; CCOc1cc(CC(=O)N[C@@H](CC(C)C)c2ccccc2N2CCCCC2)ccc1C(=O)O ; O=C(O)Cn1nnc(-c2cc(N3CCC(Oc4cc(F)ccc4Br)CC3)no2)n1]
or pharmaceutically acceptable salts thereof.

17. The inhibitor or the method of claim 4, wherein said inhibitor is an interfering oligonucleotide that inhibits expression of phosphatidylinositol-4-phosphate adaptor-2 (FAPP2).

18. The method of claim 17, wherein the interfering oligonucleotide is an siRNA or shRNA.

19. The method of claim 17, wherein the interfering oligonucleotide has a sequence that is at least 80% identical to the reverse complement of a continuous sequence of the human FAPP2 gene or a messenger RNA (mRNA) of FAPP2.

20. The method of claim 17, wherein the interfering oligonucleotide has a sequence that is at least 90% identical to the reverse complement of a continuous sequence of the human FAPP2 gene or an messenger RNA (mRNA) of FAPP2.

21. The method of claim 19, wherein the interfering oligonucleotide has a sequence that is identical to the reverse complement of a continuous sequence of the human FAPP2 gene or an messenger RNA (mRNA) of FAPP2.

22. The method of claim 17, wherein the mRNA of FAPP2 comprises one of FAPP2 mRNA Isoform 1, FAPP2 mRNA Isoform 2, and FAPP2 mRNA Isoform 3.

23. The method of claim 17, wherein the interfering oligonucleotide is less than 25 nucleotides in length.

24. The method of claim 17, wherein the interfering oligonucleotide is 16-22 nucleotides in length.

25. The method of claim 17, wherein the interfering oligonucleotide is an siRNA or shRNA having a sequence selected from:
[FAPP2.1]
SEQ ID No. 3
GAGAUAGACUGCAGCAUAU[dT][dT]
[FAPP2.2]
SEQ ID No. 4
GAAUUGAUGUGGGAACUUU[dT][dT]
[FAPP2.3]
SEQ ID No. 5
GAAAUCAACCUGUAAUACU[dT][dT]
[FAPP2.4]
SEQ ID No. 6
CCUAAGAAAUCCAACAGAA[dT][dT]
[sh FAPP2.1]
SEQ ID No. 7
CTCTTGTGGCTGAAGAGAGGTCTCAAATT;
[shFAPP2.2]
SEQ ID No. 8
TTGGCAGCCTCGATGGTTCCTTCTCTGTG;
[shFAPP2.3]-
SEQ ID No. 9
CAGTCTGGATCAGACTCAAGTTGCTCTCC;
and/or
[shFAPP2.4]
SEQ ID No. 10
TCCTGTTAAGATGGATCTTGTTGGAAATA.

26. The method of claim 17, wherein the interfering oligonucleotide comprises at least one chemical modification.

27. The method of claim 26, wherein the at least one chemical modification is selected from the group consisting of conformationary constraint nucleotide analogue, 2′O-methyl modification, phosphorothioate linkage, and combination thereof.

28. The method of claim 4, wherein the disease, disorder or condition is Fabry disease or a sphingolipidose such as Gaucher's disease.

29. The inhibitor or the method of claim 4, wherein the disease, disorder or condition is Fabry disease.

30. (canceled)

31. A method to identify a phosphatidylinositol-4-phosphate adaptor-2 (FAPP2) inhibitor comprising:
mixing acceptor vesicles, donor vesicles containing a fluorescent-labeled moiety, a quencher, and recombinant FAPP2 protein to form a mixture; and
measuring the emission intensity of the mixture either in the presence or in the absence of an agent, wherein if the emission intensity is decreased in the presence of the agent, said agent is identified as a FAPP2 inhibitor.

32. The method according to claim 31 wherein the recombinant FAPP2 protein is FAPP2-GLTP-C212 or FAPP2 Full-Length (FL).

33. The method according to claim 31, wherein the acceptor vesicles contain 1,2-dioleoyl-sn-glycero-3-phosphocholine (DOPC).


SEQUENCE LISTING (96 sequences)

SEQ ID NO: 1 - DNA, 7851 bp, Homo sapiens
ccctgggcatgcgcgagcgcgtcccgggcccggcgagtcgagggttcaggtggtgcgccgtggcgccgcctgcgaccggcagctcgttcgccgcactttggaggcttcggctgcccctccgacccacgtagggcccggacccgggcctccttgtgaacagcgtgccggcttcgccccacgggttcaccggctggctgggcttcaagcgccgaggccgccgcagtgaccccgcccccgggccgaggatgtgaggcgggccgggcgtccccacaccgggcccgggcgccgggagtgggcgtctgggcagcgccaggcgatggccctgctgctggtgctcctcgcctcttggggcctggggcagtgagggggccggcgggcgtgggccgagtggccgcgggcgccatggagggggtgctgtacaagtggaccaactatctgagcggttggcagcctcgatggttccttctctgtgggggaatattgtcctattatgattctcctgaagatgcctggaaaggttgcaaagggagcatacaaatggcagtctgtgaaattcaagttcattctgtagataatacacgcatggacctgataatccctggggaacagtatttctacctgaaggccagaagtgtggctgaaagacagcggtggctggtggccctgggatcagccaaggcttgcctgactgacagtaggacccagaaggagaaagagtttgctgaaaacactgaaaacttgaaaaccaaaatgtcagaactaagactctactgtgacctccttgttcagcaagtagataaaacaaaagaagtgaccacaactggtgtgtccaattctgaggagggaattgatgtgggaactttgctgaaatcaacctgtaatacttttctgaagaccttggaagaatgcatgcagatcgcaaatgcagccttcacctctgagctgctctaccgcactccaccaggatcacctcagctggccatgctcaagtccagcaagatgaaacatcctattataccaattcataattcattggaaaggcaaatggagttgagcacttgtgaaaatggatctttaaatatggaaataaatggtgaggaagaaatcctaatgaaaaataagaattccttatatttgaaatctgcagagatagactgcagcatatcaagtgaggaaaatacagatgataatataacagtccaaggtgaaataaggaaggaagatggaatggaaaacctgaaaaatcatgacaataacttgactcagtctggatcagactcaagttgctctccggaatgcctctgggaggaaggcaaagaagttatcccaactttctttagtaccatgaacacaagctttagtgacattgaacttctggaagacagtggcattcccacagaagcattcttggcatcatgttatgctgtggttccagtattagacaaacttggccctacagtgtttgctcctgttaagatggatcttgttggaaatattaagaaagtaaatcagaagtatataaccaacaaagaagagtttaccactctccagaagatagtgctgcacgaagtggaggcggatgtagcccaggttaggaactcagcgactgaagccctcttgtggctgaagagaggtctcaaatttttgaagggatttttgacagaagtgaaaaatggggagaaggatatccagacagccctaaataatgcatatggtaaaacattgcggcaacaccatggctgggtagttcgaggggtttttgcgttagctttaagggcagctccatcctatgaagattttgtggccgcgttaaccgtaaaggaaggtgaccaccagaaagaagctttcagtattgggatgcagagggacctcagcctttacctccctgccatggagaagcagctggccatactggacactttatatgaggtccacgggctggaatctgatgaggtggtatgatggctgctgggcagcacctcctaacttcagggaataagtgctaaagtgttttgttgccctacttaatttccagcaacagcctcaaccctctccaaccccttcacctggggggatggacaggaggtggcaaaacccagtgcttttataatttttaaaatgcatatgtgttttgtttaaagatcaaggtgctatatatttcagttcagcaggcctactggaaaccaaatgataagctgctgtagacttgaacagcaagttataagagcagatttaacaaacaaatttgctgttattgtgtattgtattgtttttatattttagtctaatgggccacccaaacccaagctgaaaatcagcaaattccatattaagtaccataattcatagccagtgtttcagccaacttagactagacatttggaggtagtataagctgctttgttgaagctgtgtagagtttgctgttcctagatgttcttcagtggaccctcttcactgcaactctgtcagtgataagggcctgtgtagtaaagatgttcagggcattcacatgaccatgcaattgtgggaggcgaagaagacgtggacaggagtcccatccttgctgacaggcatgaaaccgttgctctgagaagattaatggtgtgccctagccccaagttggaggggagaatatgagagaggtgggacaggtcatttgagatgacacctcccaactgcctaccatttaccagcatgttccccatgcattatctcaattggacatcacaagtaatgatacccagagggattattactccacttcaaaagcaaggtttagaagttgagggatctgttcacagtcacatagtttttaagcagaggagccagataatttccaagtgtgacctggactgcctctgcatcaaagtcatatggagtgcttgttcaaacagcagattcccaggccttattttggcctaaagaaccagagtctaggtggtgggacataggaatctgcatttcagtaaactttacacgtgattcttctgcacacagtattgaagagcaactagattaaattctagtttacaaaattaccagttttcttcaagaactaaatgatatgtcctttttttttttttcaaagaggataaggctgctatttaaataaaatagctaaatggagagtgagaagtggagcaggttcattcagcagcattcttaattgagccagcattgacacccagccagcaggcctttgcattgcattcggggaccatgactctgaatctgcttaccaatcaatctcggtttaatcaccaaaagtgcagagcaggcaaaatgcagctgtttatcaatctcaaaagctttgggacagtgtcatagttgaaagatgagacttaagaaaacagtttcttaaacttcttaaaacttaagaaacattgtttcataaaacaatattgagtgggcattcttctgcacagtgtgatgctccaaccctggccctagtctcagtagaccatgctgcctcgagtgtgcatcggagagaagccatgggtaccttccccattagaggctacttccttctagtaacaggaagggaagttccagcatgaggtagttatccagggtagaaggtcctttgaggggcttggttgaattgagagcatcatctctagatgatgctgttcctgctgcagatctctaggatggagagaattctctctttagtcagagaagtttatgtagggaggggtattggttttgcctttgtgtgtctttaaacaaatgaacatttatttagctcagattaattaggtattttgcccacataaagacttctggaaaatacttaaacttgaaaaatcaacatcacatgttttaaagctagggagaaagaaggggggtattaaaatgatgttgattattttgtattttgccaaggtgtgtgtgtgttatttccctcccactctcatgagcagtgagtatagatctccttctctgattagtatgaatatgatggcaggactcggggatagtccctgcccttgacatagccccctaaaacggaaaaagaaaaagccagtttttgcttgtactttgaatgaagatgttttaggcattgtaccatttagcggggatgataccaggtggttgttagaattgtgcagtgtgatcattctaaacagctgctggtgctccctgtcacctcaggtgaactctgtggtctcttggagaggtagcactctgaaaatacctcaggtttgccaccgcaactctgaatacacacaaaaggaaagctgctcagcatggccattttgcatttgtataggtagtgactagatgtacacaacttaatttgctgggaatgaggggcttaatattatctgagatcattgagaacccagatcagacagaatagcttgaataagttacattttccaattaccctttttccacatctgtagaaagagggtaatattttttaataggtattttccccactggagcatattacgtttgcctaagatgtataaaagtttgtttaagatgtgtaaaagtttgcttaaggaactgaggatccttaaataaaaatattagaaattagaaattgaacctaatactaaacagtaaattcagcttaacctgaaccttggcatagtcagagcttcctcctacatctaaagtatttgctctctgttttagttaaagtcataatttgcgctgatgtgtaatcactttccaagaagagggcaatgagaaaagatatttaaagctttctctccatagccctccaagacttctgggacaactaaatttactttcaccattactgtgagaggaggtgagaaaactctagtattttgttggcagagtaatcactttgttctcatcgctcaaagcatttttaggattatttttctagcgtaacctttagagagaactggaagaaaaaggaaattggtctactaggtattgtagacacaaataagtaacattaggctaaccccttatgagacatttccacacaatttcatcgtgcctgtacttttctctatggtaaaagccagtgtttacactttgtagggatcagggtgtatttgttgaattaaacaaaatattttcaatgatggcaagtctcttgacttttgaaagcaagtcagattccttatagctaatgctggtgaaaaatgttaaattggagagatcccttttgggagtgaaaccaaattgtaactatgaggagaagatggtcttctcattggctcttgatgtagctctgaagggagttccagaagaggagctctcacagaatgttgagcctgtgggcccaagacattgacttcgaagggtagttctcattaggatgtataagtagtggcttgaggcaccttcttatcattttttgcatgttattctgattattaaacttcccccaatgtcatattccatgatgagggatttctgaactccatagtccagcgttgttgcttttctctcttctttgctactgaaaattgccagcagtaccgccatcagcacaccaaatctacccccacttatgtttgttctgccccatttcccaggagcagcttctagcacatatgtagagtatctggcaccaccttagcccagggctgcgtgcctgatcagtggggattctgttcccccaccccccagactgcaagagcttcttaagaaggagcccatattcccatttgtagctggaaagcgggtgaatgacatgacatggggcacctaggaaagatgattattagaggagtgcagcggaaaaaaatttgcactcttctccttttggttattactttccaaatatattaacaaaaagttgatgcttttaactttatattttcagaaaagtgtttttaattaaaaatatgtgatagggaccaaataagtaaagtacatttttctccactaaatttgaagtgagggaaagaggagccaaagtaagagatttttttttaaggaaacttaatctgattgtgaaaatcatacatatggagaaacatcagatcaggcaatagagtcagagggtcatgagcaatagacgatgatgcgaggcatttggggagcttcctggaggaaaattaagtttttttcctagcaaactaccatgtcctacaagaacttggttatataatggtgcgtctctgaatcactgattaaaaccagttgcttctgattttagtcacaggttttacaagtattcagctctccctcatgtttcatttctttttttaagataatctatcaaccttttttaaattttaaaatttttagatgtagagtttataagtaaaatatatttttagccattgttctgttagctgagctgatgtgtttggtcttagagggcctgacttcagatactctttgtgatcttgtaagggctctacacaaacttcattatgtatggtaaatttgtattcttatggattgtatatagaatgctttcgttagaagtacattctacttctgtatgtccctttgtaatccgcagttgcttactcaggggtttcatagtcatttcataaaaaataattcactagctgtctaatggtattttaagactgtttatctgtatcacaacgtcattaggagttctttcaacaattccataaatatactgtttactagaccttccctgtaaatgttcccaattcccatcctgtctcagacagtcaatagtcctgtgtacagtgactatttgcatgatttctcattgcactgctgcattcaggcactccagggcatgattaaacagtcattaacagtgcctctctggtacagttgatgcatgcattgatctttcttctctgctgtttttatatagcctttaattaaaaggaaaaaaataccactactctgcaatgcaaaagtcttcaaaattctttgtttcctgtattaatcacttctgttccagagtgaacaaatgttttcagctaagctatgtgagaatgtaagaataatatcctgcttgttctaaatagttcatatatttaaagtgtggtcagtatttcctccctgtaccttacaaacagaaaccaccctgggatggttgataccccttacaaagtcgatcttacccacacagactcctgtgtatgcgtgtctgtttataggtgtatatggagtcagtgttgataggaaggatgctctagaagtacttctgttgtttcctagaaggctatgagccagttccatggcatgtttaatgtataattcccatgtatcatgagaatttcactagaatgtcattaaacagcccaactacctcatgtgaaattggctgtggacaatctgtgtcagatgagaaatgtgttcagataaatttaatctggttaatagacttaacaaattaatgtctacataaagaagaaacatgatagaccagatgccaaaggctaaaatgtacatagatttccttggattaatttttaagtcactgtttaattccatgcctagtattcttatgaatgtttgtggtttcatagatttatgcactttgaatatctgtcacgtgcagtgttaatgttacctgttcttgtctctcagcattttgaatgagcatcataatcagagtagaaggcaagttaaactataaaagtgtcaagtggcttgttaacttcttaatttaatggacctttacttagaatataatatgttggagcctcttgggaccaaccgatgagcgacagtttcatgtttagatttgtattgtttctctgtccaagtccttattctctatcttgtggggaggggtgacaggggagggttttactttttttgcaaaaatgtttgaaaatatctgtcagattttatattcgttagttataataaacttatttttaaagtattaagttcttaaaaaaaaaaaaaaaa

SEQ ID NO: 2 - protein, 519 aa, Homo sapiens
MEGVLYKWTNYLSGWQPRWFLLCGGILSYYDSPEDAWKGCKGSIQMAVCEIQVHSVDNTRMDLIIPGEQYFYLKARSVAERQRWLVALGSAKACLTDSRTQKEKEFAENTENLKTKMSELRLYCDLLVQQVDKTKEVTTTGVSNSEEGIDVGTLLKSTCNTFLKTLEECMQIANAAFTSELLYRTPPGSPQLAMLKSSKMKHPIIPIHNSLERQMELSTCENGSLNMEINGEEEILMKNKNSLYLKSAEIDCSISSEENTDDNITVQGEIRKEDGMENLKNHDNNLTQSGSDSSCSPECLWEEGKEVIPTFFSTMNTSFSDIELLEDSGIPTEAFLASCYAVVPVLDKLGPTVFAPVKMDLVGNIKKVNQKYITNKEEFTTLQKIVLHEVEADVAQVRNSATEALLWLKRGLKFLKGFLTEVKNGEKDIQTALNNAYGKTLRQHHGWVVRGVFALALRAAPSYEDFVAALTVKEGDHQKEAFSIGMQRDLSLYLPAMEKQLAILDTLYEVHGLESDEVV

SEQ ID NO: 3 - RNA, 21 nt, Artificial Sequence
  synthetic
gagauagacugcagcauaunn

SEQ ID NO: 4 - RNA, 21 nt, Artificial Sequence
  synthetic
gaauugaugugggaacuuunn

SEQ ID NO: 5 - RNA, 21 nt, Artificial Sequence
  synthetic
gaaaucaaccuguaauacunn

SEQ ID NO: 6 - RNA, 21 nt, Artificial Sequence
  synthetic
ccuaagaaauccaacagaann

SEQ ID NO: 7 - DNA, 29 bp, Artificial Sequence
  synthetic
ctcttgtggctgaagagaggtctcaaatt

SEQ ID NO: 8 - DNA, 29 bp, Artificial Sequence
  synthetic
ttggcagcctcgatggttccttctctgtg

SEQ ID NO: 9 - DNA, 29 bp, Artificial Sequence
  synthetic
cagtctggatcagactcaagttgctctcc

SEQ ID NO: 10 - DNA, 29 bp, Artificial Sequence
  synthetic
tcctgttaagatggatcttgttggaaata

SEQ ID NO: 11 - DNA, 25 bp, Artificial Sequence
  synthetic
gtgcaggctgatacgatactgcaga

SEQ ID NO: 12 - DNA, 25 bp, Artificial Sequence
  synthetic
ggatcaaggcgatgacgtggatttc

SEQ ID NO: 13 - DNA, 25 bp, Artificial Sequence
  synthetic
ccgtacagttccacaaaggcatcct

SEQ ID NO: 14 - DNA, 25 bp, Artificial Sequence
  synthetic
tggctgcagagccttgctggtaatg

SEQ ID NO: 15 - DNA, 25 bp, Artificial Sequence
  synthetic
gtccccggtgatgctgtgattgtga

SEQ ID NO: 16 - DNA, 20 bp, Artificial Sequence
  synthetic
ctcgcatggacctcatcatc

SEQ ID NO: 17 - DNA, 20 bp, Artificial Sequence
  synthetic
gatgctgcaatccacctctg

SEQ ID NO: 18 - DNA, 20 bp, Artificial Sequence
  synthetic
accacagtccatgccatcac

SEQ ID NO: 19 - DNA, 20 bp, Artificial Sequence
  synthetic
tccaccaccctgttgctgta

SEQ ID NO: 20 - DNA, 29 bp, Artificial Sequence
  synthetic
gttgaattcgatctggggataccatgtcc

SEQ ID NO: 21 - DNA, 30 bp, Artificial Sequence
  synthetic
cacctcgagcaagtacattttcatggcctc

SEQ ID NO: 22 - DNA, 33 bp, Artificial Sequence
  synthetic
caggaattcagaatgagaaggcccagcttgtta

SEQ ID NO: 23 - DNA, 35 bp, Artificial Sequence
  synthetic
aacgcggccgctgaaattcacgatcaatgcctcca

SEQ ID NO: 24 - DNA, 27 bp, Artificial Sequence
  synthetic
atagaattctggctgcagcatgcgcgc

SEQ ID NO: 25 - DNA, 31 bp, Artificial Sequence
  synthetic
cgcgatatcaagtactcgttcacctgagcca

SEQ ID NO: 26 - DNA, 1045 bp, Homo sapiens
atgtccaagccccccgacctcctgctgcggctgctccggggcgccccaaggcagcgggtctgcaccctgttcatcatcggcttcaagttcacgtttttcgtctccatcatgatctactggcacgttgtgggagagcccaaggagaaagggcagctctataacctgccagcagagatcccctgccccaccttgacaccccccaccccaccctcccacggccccactccaggcaacatcttcttcctggagacttcagaccggaccaaccccaacttcctgttcatgtgctcggtggagtcggccgccagaactcaccccgaatcccacgtgctggtcctgatgaaagggcttccgggtggcaacgcctctctgccccggcacctgggcatctcacttctgagctgcttcccgaatgtccagatgctcccgctggacctgcgggagctgttccgggacacacccctggccgcgtacccgacgggagccctacctgctgcccgtgctctccgacgcctccaggatcgcactcatgtggaagttcggcggcatctacctggacacggacttcattgttctcaagaacctgcggaacctgaccaacgtgctgggcacccagtcccgctacgtcctcaacggcgcgttcctggccttcgagcgccggcacgagttcatggcgctgtgcatgcgggacttcgtggaccactacaacggctggatctggggtcaccagggcccgcagctgctcacgcgggtcttcaagaagtggtgttccatccgcagcctggccgagagccgcgcctgccgcggcgtcaccaccctgccccctgaggccttctaccccatcccctggcaggactggaagaagtactttgaggacatcaaccccgaggagctgccgcggctgctcagtgccacctatgctgtccacgtgtggaacaagaagagccagggcacgcggttcgaggccacgtccagggcactgctggcccagctgcatgcccgctactgccccacgacgcacgaggccatgaaaatgtacttgtga

SEQ ID NO: 27 - DNA, 2262 bp, Homo sapiens
ctggggagtaatagcatgggcaaccattatcctgtctcgccgccacccaggacatggcttctgttccaatgccaagtgagtacacctatgtgaaactgagaagtgattgctcgaggccttccctgcaatggtacacccgagctcaaagcaagatgagaaggcccagcttgttattaaaagacatcctcaaatgtacattgcttgtgtttggagtgtggatcctttatatcctcaagttaaattatactactgaagaatgtgacatgaaaaaaatgcattatgtggaccctgaccatgtaaagagagctcagaaatatgctcagcaagtcttgcagaaggaatgtcgtcccaagtttgccaagacatcaatggcgctgttatttgagcacaggtatagcgtggacttactcccttttgtgcagaaggcccccaaagacagtgaagctgagtccaagtacgatcctccttttgggttccggaagttctccagtaaagtccagaccctcttggaactcttgccagagcacgacctccctgaacacttgaaagccaagacctgtcggcgctgtgtggttattggaagcggaggaatactgcacggattagaactgggccacaccctgaaccagttcgatgttgtgataaggttaaacagtgcaccagttgagggatattcagaacatgttggaaataaaactactataaggatgacttatccagagggcgcaccactgtctgaccttgaatattattccaatgacttatttgttgctgttttatttaagagtgttgatttcaactggcttcaagcaatggtaaaaaaggaaaccctgccattctgggtacgactcttcttttggaagcaggtggcagaaaaaatcccactgcagccaaaacatttcaggattttgaatccagttatcatcaaagagactgcctttgacatccttcagtactcagagcctcagtcaaggttctggggccgagataagaacgtccccacaatcggtgtcattgccgttgtcttagccacacatctgtgcgatgaagtcagtttggcgggttttggatatgacctcaatcaacccagaacacctttgcactacttcgacagtcaatgcatggctgctatgaactttcagaccatgcataatgtgacaacggaaaccaagttcctcttaaagctggtcaaagagggagtggtgaaagatctcagtggaggcattgatcgtgaattttgaacacagaaaacctcagttgaaaatgcaactctaactctgagagctgtttttgacagccttcttgatgtatttctccatcctgcagatactttgaagtgcagctcatgtttttaacttttaatttaaaaacacaaaaaaaattttagctcttcccactttttttttcctatttatttgaggtcagtgtttgtttttgcacaccattttgtaaatgaaacttaagaattgaattggaaagacttctcaaagagaattgtatgtaacgatgttgtattgatttttaagaaagtaatttaatttgtaaaacttctgctcgtttacactgcacattgaatacaggtaactaattggaaggagaggggaggtcactcttttgatggtggccctgaacctcattctggttccctgctgcgctgcttggtgtgacccacggaggatccactcccaggatgacgtgctccgtagctctgctgctgatactgggtctgcgatgcagcggcgtgaggcctgggctggttggagaaggtcacaacccttctctgttggtctgccttctgctgaaagactcgagaaccaaccagggaagctgtcctggaggtccctggtcggagagggacatagaatctgtgacctctgacaactgtgaagccaccctgggctacagaaaccacagtcttcccagcaattattacaattcttgaattccttggggattttttactgccctttcaaagcacttaagtgttagatctaacgtgttccagtgtctgtctgaggtgacttaaaaaatcagaacaaaacttctattatccagagtcatgggagagtacaccctttccaggaataatgttttgggaaacactgaaatgaaatcttcccagtattataaattgtgtatttaaaaaaaagaaacttttctgaatgcctacctggcggtgtataccaggcagtgtgccagtttaaaaagatgaaaaagaataaaaacttttgaggaaaaaaaaaaaaaaaaaaaaaaaaaaaa

SEQ ID NO: 28 - DNA, 4743 bp, Homo sapiens
cgcttcatgtggcccggcccgcgacggccggcggctgggagcggcgaggcggcggcggcggcgagtggcggcccgcgaggcccgggaggcggtggccgaggcccaggcggtggcggcggcggcccaggaggcggcggacggggagctgcgggagcaggcccggcctggctctctagcggccgcctggctgcagcatgcgcgcccgccgggggctgctgcggctgccgcgccgctcgctgctcgccgcgctcttcttcttttctctctcgtcctcgctgctgtacttcgtctatgtggcgcccggcatagtgaacacctacctcttcatgatgcaagcccaaggcattctgatccgggacaacgtgagaacaatcggtgctcaggtttatgagcaggtgcttcggagtgcttatgccaagaggaacagcagtgtaaatgactcagattatcctcttgacttgaaccacagtgaaaccttcctgcaaactacaacatttcttcctgaagacttcacctactttgcaaaccatacctgccctgaaagactcccttccatgaagggcccaatagacataaacatgagtgaaattggaatggattacattcatgaactcttctccaaagacccaaccatcaagctcggaggtcactggaagccttctgattgcatgcctcggtggaaggtggcgatccttatccccttccggaaccgccacgagcacctcccagtcctgttcagacacctgcttcccatgctccagcgccagcgcttgcagtttgcattttatgtggttgaacaagttggtacccaaccctttaatcgagccatgcttttcaacgttggctttcaagaggcaatgaaagacttggattgggactgtttgatttttcatgatgtagatcacataccggaaagtgatcgcaactattatggatgtggacagatgccgaggcattttgcaaccaaattggataagtatatgtatctgcttccttataccgagttctttggcggagtgagtggcttaacagtggaacaatttcggaaaatcaatggctttcctaatgctttctggggttggggtggagaagatgacgacctctggaacagagtacagaatgcaggctattctgtgagccggccagagggtgacacaggaaagtacaagtccattcctcatcaccatcgaggagaagtccagtttcttggaaggtatgctctgctgaggaagtcaaaagaacggcaagggctggatggcctcaacaacctgaactactttgcaaacatcacatacgacgccttgtataaaaacataactgtcaacctgacacccgagctggctcaggtgaacgagtactgagaggagagaatgtacgtttgctttacccaccgccaccaagaaagcagtccgatgagatttttttttggaggggggagggtctacacagcaagagaacagaaatactgtgtctcatgaaggatcacagagttcagggggaaaatgtgacagcacacgcacaaacgccttcactggatcagccgctggaactgagggagtgagcttggggacttccttcgtcagcactggctttctgttttcacaagacagacgtctgtcccgctgctctctccccatctcctaccccacatcctgtcttagccgcagtctccagaacccatgatgaactgtgatctgccgtggtcctgccgtggtcctgccgtggagcctgtccctacacatgaccttggagcctcttggccttcagagcagaggcaaacccaccacagggcagctgcgttttaggaagagcaaatgaaactccacaccattcttctagatctctggtgttctctttggtttcatttttttaaaaaattaccttctttgggtggggattgagggtggaggggagggtgtttgggaaagataaatagacataaatatataacaatcacttcttgaagaagtataattgtaaataagccatgtaaaatgcctttttaaaatttaattttctagctggctccaattcaaattgaggatttatgtattaggccacttacttggttggcaagtgcaggaactcagttaaaatgcagttgaagaatgtcatctcccgaattgctgtcactttggcgagggagtggatatagggcatgtcacaaaagaacaaaataacccgacctttattgctgggagctggcttctgtccctttcttcccccccccacgagtcttgcccttgacttctgctctggattcactcttccctgtcggccgcgcatgtgctcatcccactctccgctaagcgggaggctgctgttagagcaggctgcttcctgcctaaagcaggcccttcggggctcgctgcacacacatctctggctctccaggcttcgtgttctgtcttttcatcagcatggcggggcggggggcggggggcgggggtgtgtatgggaatccctccccctcttactttttctcttgtggaacttggccacagtttctgaacaatgtgcctacattaccagctggcttcagtgattcctctgtgtccctttttggtttctggaaagattctttgtcaacattagtaactgatacatagaaccaaggagcactcaaatagggagccaggagccagggagctggtgacacttgtgtgctgtggggcagctgggatccaggtaagaccggattgaagctttgaaattagactaacaaagctccagacagcaagagcccaggtgcactgctcacacccccacctgcattttgaagtcatattattttttgttttgttttttaagacggtctggctctgtcgcctaagctggagtgtggtggcacgatcacagctcactgcagcctccatctcctaggctcaagccattttcccacctcagcctcccgagtagctgggactacaggtgcacaccaccacacctggctaattttttgtatttttagtagagacaggggtttcttccatgttgcccaggctggtctcgaactcctggactcaagcaatccgcccaccttgacttcccaaagtgctgggattatgggcgggtgtgagccattgcgcccagccttgaagtcatgttctaaattgtatttgaatttgtgcctctttgtttttccccaaaccaaagccctcaaattgtagtctctgtcggcttctgcagaattctggaaaatgccagttttcctcccccgcccttgttttccataaaacatatttatatattgtgatgaggagtactttctgaagagtacttcgtatttttttttaattgccttgtttgccttcaacttccttgattttcatagtttacatgggtgtgtgtaggggtgtgtgtgtgtatgtgtgtgggttagggcttttttcgttgcatgtgatggttctgtggacatatgatccccacaaactgtgggagtgattggccaggccttgttttgtttgtttgtttgtttgtgtttttgttcttttgaagaatagagtggtatttagaaaataaattgcattgcaaagctcttatcggctcatatgagagagcaggttcctgcccttgaaaatgccggtaagctatagcatatgttttttaagacttaagcatttcatgctttaaaataccttcacaagtgaacattacacacagaagttcatttggttttcctttgttttatggtgcatatagcaataaagacccccctccaccctgcaacccccatcccccaccgggcctttgtccctgccttggcttttctccccttctcattctcctctcccctttcctcactgaaggctgtgagttgctttcaatgtgacaacactatgatgtcatttggaaggatttgccaggacagactgattctgagtcctgggtgccgtatgtgtatgcggcagtgttgtcaggcgatcttgtttgaagctctatgttgccataattaccatcaagtacacactgttggcaaaaggctaacacctgactttagaaaatgctgatttgagaacaaaaggaaaggtcttttttcactgcttaaagtggggtcactttgatacctttgcggtcatgtctgtgtctgatgagtgtagaatctctggatgtgcactgtcagtcatgtgtccaccaggcctcgaatatcatatgggaaatgtcatagttaaaaacgtacagccaggcccgtgtgctgttaatagtgtgaaattgtcatgttaaaaaaaaaaacaggaaccaaatgtgaccttgtgcatatattggtagctgaaaatcttcaaggctactgatgggtggccccttaatcttgtctttgattgctgtgtgcagggaaaggtgtccccgtttgttcatgctgttttggggggtgggggggtatttgcaagaatactcattttgacataataggtcctcttgtcagagatcctctaccacagacattaatagctgagcaggagccacatggattgattgtatccactcaccattgacgatggcattgagcgtagctagcttatttccatcactacgtgtttttgagcttgctcttacgttttaagaggtgccaggggtacatttttgcactgaaatctaaagatgttttaaaaaacacttttcacaaaaatagtcctttgtcattacattatttactcatgtgtttgtacatttttgtatgttaatttatgaatgattttttcagtaaaaaatacatattcaagaaccaaaaaaaaaaaaaaaaaa

SEQ ID NO: 29 - DNA, 4743 bp, Homo sapiens; identical to SEQ ID NO: 28

SEQ ID NO: 30 - DNA, 28 bp, Artificial Sequence
  synthetic
tctgaattcatggagggggtgctgtaca

SEQ ID NO: 31 - DNA, 34 bp, Artificial Sequence
  synthetic
tatggtaccgagcaagcaagccttggctgatccc

SEQ ID NO: 32 - DNA, 35 bp, Artificial Sequence
  synthetic
tatggtaccttgctggagggggtgctgtacaagtg

SEQ ID NO: 33 - DNA, 30 bp, Artificial Sequence
  synthetic
tcactcgagttagcaagccttggctgatcc

SEQ ID NO: 34 - DNA, 44 bp, Artificial Sequence
  synthetic
gagcatacaaatggcagtctgtgcaattcaagttcattctgtag

SEQ ID NO: 35 - DNA, 44 bp, Artificial Sequence
  synthetic
ctacagaatgaacttgaattgcacagactgccatttgtatgctc

SEQ ID NO: 36 - DNA, 1637 bp, Homo sapiens
gaggcgaaccggagcgcggggccgcggtcgccccgaccagagccgggagaccgcagcacccgcagccgcccgcgagcgcgccgaagacagcgcgcaggcgagagcgcgcgggcgggggcgcgcaggccctgcccgccccttccgtccccacccccctccgccctttcctctccccaccttcctctcgcctcccgcgcccccgcaccgggcgcccaccctgtcctcctcctgcgggagcgttgtccgtgttggcggccgcagcgggccgggccggtccggcgggccgggggatggcgctgctggacctggccttggagggaatggccgtcttcgggttcgtcctcttcttggtgctgtggctgatgcatttcatggctatcatctacacccgattacacctcaacaagaaggcaactgacaaacagccttatagcaagctcccaggtgtctctcttctgaaaccactgaaaggggtagatcctaacttaatcaacaacctggaaacattctttgaattggattatcccaaatatgaagtgctcctttgtgtacaagatcatgatgatccagccattgatgtatgtaagaagcttcttggaaaatatccaaatgttgatgctagattgtttataggtggtaaaaaagttggcattaatcctaaaattaataatttaatgccaggatatgaagttgcaaagtatgatcttatatggatttgtgatagtggaataagagtaattccagatacgcttactgacatggtgaatcaaatgacagaaaaagtaggcttggttcacgggctgccttacgtagcagacagacagggctttgctgccaccttagagcaggtatattttggaacttcacatccaagatactatatctctgccaatgtaactggtttcaaatgtgtgacaggaatgtcttgtttaatgagaaaagatgtgttggatcaagcaggaggacttatagcttttgctcagtacattgccgaagattactttatggccaaagcgatagctgaccgaggttggaggtttgcaatgtccactcaagttgcaatgcaaaactctggctcatattcaatttctcagtttcaatccagaatgatcaggtggaccaaactacgaattaacatgcttcctgctacaataatttgtgagccaatttcagaatgctttgttgccagtttaattattggatgggcagcccaccatgtgttcagatgggatattatggtatttttcatgtgtcattgcctggcatggtttatatttgactacattcaactcaggggtgtccagggtggcacactgtgtttttcaaaacttgattatgcagtcgcctggttcatccgcgaatccatgacaatatacatttttttgtctgcattatgggacccaactataagctggagaactggtcgctacagattacgctgtgggggtacagcagaggaaatcctagatgtataactacagctttgtgactgtatataaaggaaaaaagagaagtattataaattatgtttatataaatgcttttaaaaatctaccttctgtagttttatcacatgtatgttttggtatctgttctttaatttatttttgcatggcacttgcatctgtgaaaaaaaa

SEQ ID NO: 37 - DNA, 1333 bp, Homo sapiens
gcgggaggccgcgcaggcgcagtgagtcagtgccggtcggtcttgtgggctgaggggcagcggcttaggctccggcgtctgcaggggtcgccgagctaacccgtggctaggcgagtggggcggggcggccggcaccatgtcgaggcaggcgaaccgtggcaccgagagcaagaaaatgagctctgagctcttcaccctgacctatggtgccctggtcacccagctatgtaaggactatgaaaatgatgaagatgtgaataaacagctggacaaaatgggctttaacattggagtccggctgattgaagatttcttggctcggtcaaatgttgggaggtgccatgactttcgggaaactgcggatgtcattgccaaggtggcgttcaagatgtacttgggcatcactccaagcattactaattggagcccagctggtgatgaattctccctcattttggaaaataaccccttggtggactttgtggaacttcctgataaccactcatcccttatttattccaatctcttgtgtggggtgttgcggggagctttggagatggtccagatggctgtggaggccaagtttgtccaggacaccctgaaaggagacggtgtgacagaaatccggatgagattcatcaggcggattgaggacaatcttccagctggagaggaataaccatccctacaactcgaggatagccatcaggagcactgttggaatcagcaggcctctgtgctccctctgccctccagaactcagtgactcttgaacatggatgttatatattcttataacctgtttccattctccattcaaataaagagcagactgcgatatagtccatttaccccatgtgtgcacattcaggagcgacagtctctgcccccattcccttgagaggggctggatgtaatcacctttggttggactagaaagagctcaaaccattttacattcctgtttgaatttttccaaagcaaaactcactttgaccccattaagaggcaagcctggcacatctatccctgggcctttagaaagccatttgcctcaaatggctatagggttgtggggtggagggaggaagggctgggagggagtggggaggaattgctagctgtagtgtgacacattgtagtgtttgccaggaaaggagccagtcatgccggaaacactgacttctgggaagccacccaggtctcattcctccctgctgttggaggcaacatctcctctttttacagagggtacatccttttttcttacaaattcttcaataaagacacattcttgagtgaaatcccaatcaaaaaaaaaaaaaaaaaaaaa

SEQ ID NO: 38 - DNA, 4743 bp, Homo sapiens; identical to SEQ ID NO: 28

SEQ ID NO: 39 - DNA, 4814 bp, Homo sapiens
ccggctttcttccgtcctgccgtccccgggcccctagcccccagtcccctctcccccctccacctcttgtccctcggcctccgcgcctcccctcccccgactctctggcctcccattcattccgtccgctccctcccatttcctcttttccgctcgggccctctccccagagcctccccgcgcggaccccgctgcagtctcggggttctcagcacatttatggaagtttagggcctggacagtggccccgagtccggcctgagagcgcagcctgggctgctggcagggaagaggaagatgtctgtgctcaggcggatgatgcgggtttccaatcgctctctcctcgccttcatcttcttcttctccctctcttcgtcctgtctgtacttcatctatgtggccccaggcatcgccaacacatatctctttatggtacaagctcgaggtataatgttgagagaaaatgtgaaaacaataggtcatatgatcaggctgtacacaaataaaaacagtacgctcaacggtacagattatcccgaaggcaataattcaagtgattatcttgttcaaacaacaacgtatctcccggaaaacttcacatactcaccatacctcccctgtccagaaaagctgccttatatgcgaggattcctcaatgtcaatgtaagcgaagtcagttttgatgaaattcatcaactcttctccaaggatttagatattgagccagggggtcattggaggccaaaagactgtaaacccagatggaaggtggcagttctcattcctttccgtaatcgccatgaacatcttccaatttttttcttacatctgattccaatgctccagaagcagcggctggaatttgcgttttatgtcattgaacagactggcacacaaccttttaaccgtgcgatgcttttcaatgtgggcttcaaagaggccatgaaagacagtgtctgggactgtgtaatcttccacgatgtggatcatctacctgaaaatgaccggaactattacggatgtggagaaatgccacgtcattttgctgcaaagctggataaatacatgtatattcttccatataaagaattttttggtggtgtaagtgggctgacagtggaacaatttagaaagatcaatggttttcctaatgccttctggggatggggaggagaagatgatgacctttggaacagagttcactatgctggatataatgtaaccagaccagagggagacttaggaaaatacaagtcaattcctcatcaccatagaggtgaagtccagtttttaggacggtataaattactaaggtattccaaggagcgtcagtacatcgatggactgaacaatttaatatataggccaaaaatactggttgataggttgtatacaaacatatctgtaaacctcatgccagagttagctccaatcgaagactattaaaagaagtggctgtcgtggcaaggtagaccacaatgctggatcataaacttggagcgcgctcttagtggaggtcagtgattggctgtgtcacagtgcccttttctctgagaagagccagcagtccacagtgtttacagagtgggactatatacagtcacccttctctcacccgtcctccctgctctgagacacacccctgtggcgagcaccgcagaaacgggaagccactacttaagatcggaagttaagagagctccctccgaagagaaaatttttatactaaaatctataatttaattcaagagaatgcttttatttccgtttaaacatattttgtatatatgtgatataaattaatgtgtgcaaattgtttaaaaattagatgtgttgcagttttgcatgtaatcggttatacctttattggacttttatagacattttttatttgcatgaaaaaaactcactaaatttacatcactaaacaaaggttaacccttgtgtgaaatgaaggaactgtcaataattgacagccaactaatacagtaaactgttatactagttttgagctttagacctcagccttttgtgtggaagaagtcacagctttcttaggctttaaaggaaaagaaggaaggacttaaatagcttttcttcctaccgggattacctatgtttttccttgcttgcaatctcatctgattttgctagaaatcacaaccatattgtttatgcatattgcatgagtattaccaagaaaaatcttaaaagttgtgatgtgacatgatataaaggatctctttatgttaaatgtctttccatgtacctctggtgtgtcagggattttgtgcctcaaaaaatgtttccaaggttgtgtgtttatactgtgtattttttttaaattcacggtgaacagcacttttattatttccagttcagaagagccaaaaaaaagtatcttcatttaaaaagaaatcaagtcagtttttttaaaagaatgatctatggaagaaaacccaatagtacttataatatacaaatgaattatactattagataagaattaaatattctatttttataaagataaatacttagtagataaaaacaaattttaagtttagttttcagacatagaatttcacttttggaggggacccttgaatgttagtttcattcaaactatattttgtttttttcacttttaagagattatatctctggtgttaattactaaaattttctttctaaaagtataatttaatagaattatcttaaaatacatgatcagtattcagtgcgctgatattataatgtcttctatatgtagtatctaaaagcataatttaatagcattgtcttaaaatacgtgatgagttatttggtgcactgatgccataatgtcttctatatgtaatatcttacatgtttgcatgagtgtcagggctttgtccagttatcttcatatgactgttctctgaaaaaaggtcttactacctgatatcaagtacatatcttaattttggatgaatttttccacagtaaagccgaacagtattcttttttgaaaattagcaattataaagattgtttaaaatatcttgatattttcacaaattttaaacaaaaaattaaatatctataaagtgaatgttcaacaacaatgatgagcaatagaaatctatatatgagaaacgtttggataatatttgcctcaaggattgtttacctaaaacagaactatggttatttatctcagattgtaagttttattttgaagtgtcatttttatggcaatattgataacacaaatgaccacaaactaaatcataaaataattgaatccttacactttgagaaaatggatagcttctgtgttatgataccaaacaaaatattttcatatttatttcttagaactatttccaaaatatttatatttcagtttttttatggtactcgaaaaatcaaaccttctgaaaattatgatatttgtacttcatttgtgtattagttcattttctgtttctattacatagtttttaaagtcaatgaaagagattgagaatgttaacatttgttgatcaatgtggagctgtccacattcggtatcttttcatttttatatatattttacagtttcaagaaaacttagtactactagtatctgccttattttcttaagtctgatgttaatttttgtttcaggagtgagatataaacattttgctttttttcagttttgaaatatagtttgtcatcctgtgaaaaccacctcgtttgctttttgtttgtttgttttgcttttttagtgtagacctaaaattatttttgcctataaacagtgaacttaatatgtttctgtgagagtgacagagctgtgcccgtttatcttcattgtattaattattacataaagattaacaaatacattgcaaatgcaaattataggatgtttggaggatttattaatgagcatgtattgattgcctattccatatccagtgcaatgctaggtatgtctatggaaggagtattattacttcatttctaaacaaatgaagtcaagcaaattgagatctttctgaaattatgaagacatcttctgataacttatctgttgtatttcctagtctcaaagtcaagaaacccgtatatgtggacctagggctaataggaggtccctatggcactaactgggaaaattacatggtgcttttcaactacagaaagaagtaatgactaatgggcctgtctgtagagaagaaaaggagaagagtacacatttgctggaactatatataattcacagtggagatagttaattctttgctaaaactagggttttatgagtattttttaaatctacatttaagtttttgtttttgtgaaaagttattcagtaagaaaactttataatattaagtagatacagttatgtattaaaaatttccttatatacccttttgccagccaagatactgatattttaaaacaaaatggaaaaatgtccaatacaatattctctttctggaggcttcctacaagtgaaagcagattttgtcattttatttggcctttgcttgtctttgatatatttctttatgtgtttttcaaatacatactgttgtagatttattgtcttgtatttgcaagtatgccggttgagagaactaaaatggttatagtattgtttatcagaaagaggaagtgtacttaagattattttatttatttagatgaaaggcttctacataaatgcactgaaataaatgctagtaatttcttgtatcccacttctttttttgcttctcatggaaatgtttgtacttttttatcattgtcttttatgaaatataatgttctaaaga

SEQ ID NO: 40 - DNA, 2262 bp, Homo sapiens; identical to SEQ ID NO: 27

SEQ ID NO: 41 - DNA, 2106 bp, Homo sapiens
actgccctccggccgccgcgccgcccgcccgccggggccccgctgtccgccgcccgccgccgctggagctagagatggatttgcagccgctgcaagtgtgtggaagggccgtgttcgtgttggcaaagaaggtcggctgctgagccagggcgtgtctcccggaggcctgtgggctgccaggatccccacctctctgcaatgggctgcccaggctgaccagccggttcctgctggaagctcctggtctgatctggggataccatgtccaagccccccgacctcctgctgcggctgctccggggcgccccaaggcagcgggtctgcaccctgttcatcatcggcttcaagttcacgtttttcgtctccatcatgatctactggcacgttgtgggagagcccaaggagaaagggcagctctataacctgccagcagagatcccctgccccaccttgacaccccccaccccaccctcccacggccccactccaggcaacatcttcttcctggagacttcagaccggaccaaccccaacttcctgttcatgtgctcggtggagtcggccgccagaactcaccccgaatcccacgtgctggtcctgatgaaagggcttccgggtggcaacgcctctctgccccggcacctgggcatctcacttctgagctgcttcccgaatgtccagatgctcccgctggacctgcgggagctgttccgggacacacccctggccgactggtacgcggccgtgcaggggcgctgggagccctacctgctgcccgtgctctccgacgcctccaggatcgcactcatgtggaagttcggcggcatctacctggacacggacttcattgttctcaagaacctgcggaacctgaccaacgtgctgggcacccagtcccgctacgtcctcaacggcgcgttcctggccttcgagcgccggcacgagttcatggcgctgtgcatgcgggacttcgtggaccactacaacggctggatctggggtcaccagggcccgcagctgctcacgcgggtcttcaagaagtggtgttccatccgcagcctggccgagagccgcgcctgccgcggcgtcaccaccctgccccctgaggccttctaccccatcccctggcaggactggaagaagtactttgaggacatcaaccccgaggagctgccgcggctgctcagtgccacctatgctgtccacgtgtggaacaagaagagccagggcacgcggttcgaggccacgtccagggcactgctggcccagctgcatgcccgctactgccccacgacgcacgaggccatgaaaatgtacttgtgaggggcccgccaggtcacctccccaacctgctcctgatggggcactgggccgcccttcccggggaggcaagattgagggcccgggagagggaggcccgagctgccaccgggcttaggcaggctgttgaggagctgtgggagcaggcccagtgggaggctgtggacaccccgaggacagtgtcctgtctcgaggcagggctgacacatggtgccatagccagcggagggcgctcagtgagtgccccgggccttctagacaacaggcaggaaggatgaacctcagggcacccccaggtggtgcggaaagccaggcagttgggacagaggtgcccacgagggcagaggccggtgctaaggggatggggaagaagggacaagattcccagagaggagaggaggctgttggtaggaaagtggcagggctgggggagacccagccccaagggtccggggcggaggatgctttgttcttttctggttttggttcctctttcgcggggggtgggggaggtcaacagggactgagtggggcagaggcccagaagtgccagcctggggagccgtttgggggcagccccttctgcccaccccatccttcttcctctccagagatgccaggggggcgtgtatgctctgccccttccctcagacaggggctgggtggggaggctctttaggctcaggagaagcattttaaagaaacccccaccctgccgcccgcattataaacacaggagaataatcaatagaataaaagtgaccgactgtcaaaaaaaaaaaaaaa

SEQ ID NO: 42 - DNA, 3032 bp, Homo sapiens
tttgtttgcggaagtaggaggaagtagaagtgctgagtaagccgagacagagggggaagacggtggggcctccccacctgccccgcagaagatgcagttctttggccgcctggtcaataccttcagtggcgtcaccaacttgttctctaacccattccgggtgaaggaggtggctgtggccgactacacctcgagtgaccgagttcgggaggaagggcagctgattctgttccagaacactcccaaccgcacctgggactgcgtcctggtcaaccccaggaactcacagagtggattccgactcttccagctggagttggaggctgacgccctagtgaatttccatcagtattcttcccagctgctacccttctatgagagctcccctcaggtcctgcacactgaggtcctgcagcacctgaccgacctcatccgtaaccaccccagctggtcagtggcccacctggctgtggagctagggatccgcgagtgcttccatcacagccgtatcatcagctgtgccaattgcgcggagaacgaggagggctgcacacccctgcacctggcctgccgcaagggtgatggggagatcctggtggagctggtgcagtactgccacactcagatggatgtcaccgactacaagggagagaccgtcttccattatgctgtccagggtgacaattctcaggtgctgcagctccttggaaggaacgcagtggctggcctgaaccaggtgaataaccaagggctgaccccgctgcacctggcctgccagctggggaagcaggagatggtccgcgtgctgctgctgtgcaatgctcggtgcaacatcatgggccccaacggctaccccatccactcggccatgaagttctctcagaaggggtgtgcggagatgatcatcagcatggacagcagccagatccacagcaaagacccccgttacggagccagccccctccactgggccaagaacgcagagatggcccgcatgctgctgaaacggggctgcaacgtgaacagcaccagctccgcggggaacacggccctgcacgtggcggtgatgcgcaaccgcttcgactgtgccatagtgctgctgacccacggggccaacgcggatgcccgcggagagcacggcaacaccccgctgcacctggccatgtcgaaagacaacgtggagatgatcaaggccctcatcgtgttcggagcagaagtggacaccccgaatgactttggggagactcctacattcctagcctccaaaatcggcagacaactacaggatctcatgcacatctcacgggcccggaagccagcgttcatcctgggctccatgagggacgagaagcggacccacgaccacctgctgtgcctggatggaggaggagtgaaaggcctcatcatcatccagctcctcatcgccatcgagaaggcctcgggtgtggccaccaaggacctgtttgactgggtggcgggcaccagcactggaggcatcctggccctggccattctgcacagtaagtccatggcctacatgcgcggcatgtactttcgcatgaaggatgaggtgttccggggctccaggccctacgagtcggggcccctggaggagttcctgaagcgggagtttggggagcacaccaagatgacggacgtcaggaaacccaaggtgatgctgacagggacactgtctgaccggcagccggctgaactccacctcttccggaactacgatgctccagaaactgtccgggagcctcgtttcaaccagaacgttaacctcaggcctccagctcagccctcagaccagctggtgtggcgggcggcccgaagcagcggggcagctcctacttacttccgacccaatgggcgcttcctggacggtgggctgctggccaacaaccccacgctggatgccatgaccgagatccatgagtacaatcaggacctgatccgcaagggtcaggccaacaaggtgaagaaactctccatcgttgtctccctggggacagggaggtccccacaagtgcctgtgacctgtgtggatgtcttccgtcccagcaacccctgggagctggccaagactgtttttggggccaaggaactgggcaagatggtggtggactgttgcacggatccagacgggcgggctgtggaccgggcacgggcctggtgcgagatggtcggcatccagtacttcagattgaacccccagctggggacggacatcatgctggatgaggtcagtgacacagtgctggtcaacgccctctgggagaccgaggtctacatctatgagcaccgcgaggagttccagaagctcatccagctgctgctctcaccctgagggtccccagcctctcaccggccccagctgacctcgtccattcagcccctgccaggccaagcccagccactgccctcccgggcagatctgggcccaggcacctctgagtccatagaccaggcctgggagaatgccaagctgcctgcccgaggctggtcctgaaggcctgtctcccactaaccccgccttccagcactttctgtcattccaggctgggaaagtctagagccccctttggcccctttccctgactgtcaaggacaactgactcccccatcagctcaaacattaagggtacccgggcacaaccgtacccctgcccccagccccagcctccctgagggcctgccgggctgcctctgccccagcccccagcaagggcactcccaggcttcctggtgggtgcagcccactccctctgccctctgctccgttccctgggggctgggactaaagaaatgggtgtcccccaccccatcagctgggaaagcccaggccgcaggagtgggatgcccgttggactttgcccctcacactggcccagcccctcacactgccccaccccgagaaccctcagctctcaaaggtcactcctgggagtttcttcttcccaatggaagtggcttaagagccaaaactgaaataaatcatttggattcaagttcaaaaaaaaaaaaaaaaa

SEQ ID NO: 43 - RNA, 21 nt, Artificial Sequence
  synthetic
gauaugaaguugcaaaguann

SEQ ID NO: 44 - RNA, 21 nt, Artificial Sequence
  synthetic
gcgaauccaugacaauauann

SEQ ID NO: 45 - RNA, 21 nt, Artificial Sequence
  synthetic
ggaccaaacuacgaauuaann

SEQ ID NO: 46 - RNA, 21 nt, Artificial Sequence
  synthetic
gaugcuagauuguuuauagnn

SEQ ID NO: 47 - RNA, 21 nt, Artificial Sequence
  synthetic
gugaaauuggaauggauuann

SEQ ID NO: 48 - RNA, 21 nt, Artificial Sequence
  synthetic
gcuuaacaguggaacaauunn

SEQ ID NO: 49 - RNA, 21 nt, Artificial Sequence
  synthetic
ggaaagugaucgcaacuaunn

SEQ ID NO: 50 - RNA, 21 nt, Artificial Sequence
  synthetic
cauaucucuuuaugguacann

SEQ ID NO: 51 - RNA, 21 nt, Artificial Sequence
  synthetic
cuccaaucgaagacuauuann

SEQ ID NO: 52 - RNA, 21 nt, Artificial Sequence
  synthetic
gguauuccaaggagcgucann

SEQ ID NO: 53 - RNA, 21 nt, Artificial Sequence
  synthetic
caauggcgcuguuauuugann

SEQ ID NO: 54 - RNA, 21 nt, Artificial Sequence
  synthetic
gaccaugcauaaugugacann

SEQ ID NO: 55 - RNA, 21 nt, Artificial Sequence
  synthetic
cggaaguucuccaguaaagnn

SEQ ID NO: 56 - RNA, 21 nt, Artificial Sequence
  synthetic
aggaauacugcacggauuann

SEQ ID NO: 57 - RNA, 21 nt, Artificial Sequence
  synthetic
agaaagggcagcucuauaann

SEQ ID NO: 58 - RNA, 21 nt, Artificial Sequence
  synthetic
ggacacggacuucauuguunn

SEQ ID NO: 59 - RNA, 21 nt, Artificial Sequence
  synthetic
ugaaagggcuuccggguggnn

SEQ ID NO: 60 - RNA, 21 nt, Artificial Sequence
  synthetic
gcacucauguggaaguucgnn

SEQ ID NO: 61 - RNA, 21 nt, Artificial Sequence
  synthetic
ggacagucguuaagaaguann

SEQ ID NO: 62 - RNA, 21 nt, Artificial Sequence
  synthetic
ggagaaacacuaauucauann

SEQ ID NO: 63 - RNA, 21 nt, Artificial Sequence
  synthetic
ggagaagacuuucagacaann

SEQ ID NO: 64 - RNA, 21 nt, Artificial Sequence
  synthetic
guacaaggcuccagguguunn

SEQ ID NO: 65 - RNA, 21 nt, Artificial Sequence
  synthetic
gauaaccacucaucccuuann

SEQ ID NO: 66 - RNA, 21 nt, Artificial Sequence
  synthetic
gggcaucacuccaagcauunn

SEQ ID NO: 67 - RNA, 21 nt, Artificial Sequence
  synthetic
gaaaggagacggugugacann

SEQ ID NO: 68 - RNA, 21 nt, Artificial Sequence
  synthetic
ggaaacugcggaugucauunn

SEQ ID NO: 69 - DNA, 20 bp, Artificial Sequence
  synthetic
aagagctacgagctgcctga

SEQ ID NO: 70 - DNA, 19 bp, Artificial Sequence
  synthetic
gactccatgcccaggaagg

SEQ ID NO: 71 - DNA, 20 bp, Artificial Sequence
  synthetic
tgctgacctgctggattaca

SEQ ID NO: 72 - DNA, 19 bp, Artificial Sequence
  synthetic
cctgacaaggaaagcaaag

SEQ ID NO: 73 - DNA, 21 bp, Artificial Sequence
  synthetic
caatcggtgctcaggtttatg

SEQ ID NO: 74 - DNA, 21 bp, Artificial Sequence
  synthetic
ggtttcactgtggttcaagtc

SEQ ID NO: 75 - DNA, 20 bp, Artificial Sequence
  synthetic
tcctaagtctccctctggtc

SEQ ID NO: 76 - DNA, 21 bp, Artificial Sequence
  synthetic
acgtatctcccggaaaacttc

SEQ ID NO: 77 - DNA, 20 bp, Artificial Sequence
  synthetic
ggagggggtgctgtacaagt

SEQ ID NO: 78 - DNA, 20 bp, Artificial Sequence
  synthetic
ggacaatattcccccacaga

SEQ ID NO: 79 - DNA, 20 bp, Artificial Sequence
  synthetic
gatccccacctctctgcaat

SEQ ID NO: 80 - DNA, 20 bp, Artificial Sequence
  synthetic
ttggacatggtatccccaga

SEQ ID NO: 81 - DNA, 19 bp, Artificial Sequence
  synthetic
tggttattggaagcggagg

SEQ ID NO: 82 - DNA, 22 bp, Artificial Sequence
  synthetic
tctgaatatccctcaactggtg

SEQ ID NO: 83 - DNA, 18 bp, Artificial Sequence
  synthetic
ttcgggttcgtcctcttc

SEQ ID NO: 84 - DNA, 22 bp, Artificial Sequence
  synthetic
gcttgctataaggctgtttgtc

SEQ ID NO: 85 - DNA, 2147 bp, Homo sapiens
ccctgggcatgcgcgagcgcgtcccgggcccggcgagtcgagggttcaggtggtgcgccgtggcgccgcctgcgaccggcagctcgttcgccgcactttggaggcttcggctgcccctccgacccacgtagggcccggacccgggcctccttgtgaacagcgtgccggcttcgccccacgggttcaccggctggctgggcttcaagcgccgaggccgccgcagtgaccccgcccccgggccgaggatgtgaggcgggccgggcgtccccacaccgggcccgggcgccgggagtgggcgtctgggcagcgccaggcgatggccctgctgctggtgctcctcgcctcttggggcctggggcagtgagggggccggcgggcgtgggccgagtggccgcgggcgccatggagggggtgctgtacaagtggaccaactatctgagcggttggcagcctcgatggttccttctctgtgggggaatattgtcctattatgattctcctgaagatgcctggaaaggttgcaaagggagcatacaaatggcagtctgtgaaattcaagttcattctgtagataatacacgcatggacctgataatccctggggaacagtatttctacctgaaggccagaagtgtggctgaaagacagcggtggctggtggccctgggatcagccaaggcttgcctgactgacagtaggacccagaaggagaaagagtttgctgaaaacactgaaaacttgaaaaccaaaatgtcagaactaagactctactgtgacctccttgttcagcaagtagataaaacaaaagaagtgaccacaactggtgtgtccaattctgaggagggaattgatgtgggaactttgctgaaatcaacctgtaatacttttctgaagaccttggaagaatgcatgcagatcgcaaatgcagccttcacctctgagctgctctaccgcactccaccaggatcacctcagctggccatgctcaagtccagcaagatgaaacatcctattataccaattcataattcattggaaaggcaaatggagttgagcacttgtgaaaatggatctttaaatatggaaataaatggtgaggaagaaatcctaatgaaaaataagaattccttatatttgaaatctgcagagatagactgcagcatatcaagtgaggaaaatacagatgataatataacagtccaaggtgaaataaggaaggaagatggaatggaaaacctgaaaaatcatgacaataacttgactcagtctggatcagactcaagttgctctccggaatgcctctgggaggaaggcaaagaagttatcccaactttctttagtaccatgaacacaagctttagtgacattgaacttctggaagacagtggcattcccacagaagcattcttggcatcatgttatgctgtggttccagtattagacaaacttggccctacagtgtttgctcctgttaagatggatcttgttggaaatattaagaaagtaaatcagaagtatataaccaacaaagaagagtttaccactctccagaagatagtgctgcacgaagtggaggcggatgtagcccaggttaggaactcagcgactgaagccctcttgtggctgaagagaggtctcaaatttttgaagggatttttgacagaagtgaaaaatggggagaaggatatccagacagccctaaataatgcatatggtaaaacattgcggcaacaccatggctgggtagttcgaggggtttttgcgggacacacaaatgtctgatggtggcccaggagggctgtcactcagaatcaggcgggtgcagagtggcctgcaggacatgatgacccagcatgagacaggatgcatcacatctgggtgacgcctctctccaggggatcagcctggactaaatgcctctcctgggccaggatgcccagcaagccccgagatgcctcctggagagaaggaagtatgtgagagtatcctgagttaagtgagatctaaaatgcctgagaaaaccctgaaatgactgagtctatctggaacttatttgattgtttttctgccatcagatagaatgaggtctcaaaagagaaattaaattatattacaagaaaataaagctcaattgcttgccaaaaaaa

SEQ ID NO: 86 - protein, 459 aa, Homo sapiens
MEGVLYKWTNYLSGWQPRWFLLCGGILSYYDSPEDAWKGCKGSIQMAVCEIQVHSVDNTRMDLIIPGEQYFYLKARSVAERQRWLVALGSAKACLTDSRTQKEKEFAENTENLKTKMSELRLYCDLLVQQVDKTKEVTTTGVSNSEEGIDVGTLLKSTCNTFLKTLEECMQIANAAFTSELLYRTPPGSPQLAMLKSSKMKHPIIPIHNSLERQMELSTCENGSLNMEINGEEEILMKNKNSLYLKSAEIDCSISSEENTDDNITVQGEIRKEDGMENLKNHDNNLTQSGSDSSCSPECLWEEGKEVIPTFFSTMNTSFSDIELLEDSGIPTEAFLASCYAVVPVLDKLGPTVFAPVKMDLVGNIKKVNQKYITNKEEFTTLQKIVLHEVEADVAQVRNSATEALLWLKRGLKFLKGFLTEVKNGEKDIQTALNNAYGKTLRQHHGWVVRGVFAGHTNV

SEQ ID NO: 87 - DNA, 2315 bp, Homo sapiens
ccctgggcatgcgcgagcgcgtcccgggcccggcgagtcgagggttcaggtggtgcgccgtggcgccgcctgcgaccggcagctcgttcgccgcactttggaggcttcggctgcccctccgacccacgtagggcccggacccgggcctccttgtgaacagcgtgccggcttcgccccacgggttcaccggctggctgggcttcaagcgccgaggccgccgcagtgaccccgcccccgggccgaggatgtgaggcgggccgggcgtccccacaccgggcccgggcgccgggagtgggcgtctgggcagcgccaggcgatggccctgctgctggtgctcctcgcctcttggggcctggggcagtgagggggccggcgggcgtgggccgagtggccgcgggcgccatggagggggtgctgtacaagtggaccaactatctgagcggttggcagcctcgatggttccttctctgtgggggaatattgtcctattatgattctcctgaagatgcctggaaaggttgcaaagggagcatacaaatggcagtctgtgaaattcaagttcattctgtagataatacacgcatggacctgataatccctggggaacagtatttctacctgaaggccagaagtgtggctgaaagacagcggtggctggtggccctgggatcagccaaggcttgcctgactgacagtaggacccagaaggagaaagagtttgctgaaaacactgaaaacttgaaaaccaaaatgtcagaactaagactctactgtgacctccttgttcagcaagtagataaaacaaaagaagtgaccacaactggtgtgtccaattctgaggagggaattgatgtgggaactttgctgaaatcaacctgtaatacttttctgaagaccttggaagaatgcatgcagatcgcaaatgcagccttcacctctgagctgctctaccgcactccaccaggatcacctcagctggccatgctcaagtccagcaagatgaaacatcctattataccaattcataattcattggaaaggcaaatggagttgagcacttgtgaaaatggatctttaaatatggaaataaatggtgaggaagaaatcctaatgaaaaataagaattccttatatttgaaatctgcagagatagactgcagcatatcaagtgaggaaaatacagatgataatataacagtccaaggtgaaataaggaaggaagatggaatggaaaacctgaaaaatcatgacaataacttgactcagtctggatcagactcaagttgctctccggaatgcctctgggaggaaggcaaagaagttatcccaactttctttagtaccatgaacacaagctttagtgacattgaacttctggaagacagtggcattcccacagaagcattcttggcatcatgttatgctgtggttccagtattagacaaacttggccctacagtgtttgctcctgttaagatggatcttgttggaaatattaagaaagtaaatcagaagtatataaccaacaaagaagagtttaccactctccagaagatagtgctgcacgaagtggaggcggatgtagcccaggttaggaactcagcgactgaagccctcttgtggctgaagagaggtctcaaatttttgaagggatttttgacagaagtgaaaaatggggagaaggatatccagacagccctaagaaatccaacagaaaacacttgacaccaaaacataccctgatgaagatcctgaacttcaagaatgaagaaagaattcctcaccattcaggcagaaaaagcaagtcaccaagggacctcaaacttcctttccacaagattctgtgacgggaaacaatgggggagtatttccgaagttctgagtaggaaaaaagaatgactcaaatgtattattgccaaccaagtcgtcaaatctaatgtcaagttctcttaagcaggtaagaactcagaacataatacctgagtgccttcttaaggaaaccatttgataggaaagatgaaccaaataactcaatgatggatgagctggtagaaaaaaagctggtggtgaaccaaggtcaaactggaaattatagtcacagtatagatatagattataaatattacaaaccctaagatagctaataaattgggaatgggagaagggaggatataagagcactaatgccctcttattttcatagcagagacttgatactgtctcaacttttttcaaaaacacaatttcttaaattttttggtaatcttttaaataaacagatttctaaaaagaaaaaaaaaaaaaaaaaaaaaaaa

SEQ ID NO: 88 - protein, 440 aa, Homo sapiens
MEGVLYKWTNYLSGWQPRWFLLCGGILSYYDSPEDAWKGCKGSIQMAVCEIQVHSVDNTRMDLIIPGEQYFYLKARSVAERQRWLVALGSAKACLTDSRTQKEKEFAENTENLKTKMSELRLYCDLLVQQVDKTKEVTTTGVSNSEEGIDVGTLLKSTCNTFLKTLEECMQIANAAFTSELLYRTPPGSPQLAMLKSSKMKHPIIPIHNSLERQMELSTCENGSLNMEINGEEEILMKNKNSLYLKSAEIDCSISSEENTDDNITVQGEIRKEDGMENLKNHDNNLTQSGSDSSCSPECLWEEGKEVIPTFFSTMNTSFSDIELLEDSGIPTEAFLASCYAVVPVLDKLGPTVFAPVKMDLVGNIKKVNQKYITNKEEFTTLQKIVLHEVEADVAQVRNSATEALLWLKRGLKFLKGFLTEVKNGEKDIQTALRNPTENT

SEQ ID NO: 89 - DNA, 7851 bp, Homo sapiens; identical to SEQ ID NO: 1

SEQ ID NO: 90 - RNA, 21 nt, Artificial Sequence
  synthetic
gcuaucauggcugcuccuunn

SEQ ID NO: 91 - RNA, 21 nt, Artificial Sequence
  synthetic
gcaaucacuggcgaaauuunn

SEQ ID NO: 92 - RNA, 21 nt, Artificial Sequence
  synthetic
cagcuuagacagggagacann

SEQ ID NO: 93 - DNA, 29 bp, Artificial Sequence
  synthetic
cctgaataggactggcagaagcattgtgt

SEQ ID NO: 94 - DNA, 29 bp, Artificial Sequence
  synthetic
gaagagccagattcctgcatcagtgagaa

SEQ ID NO: 95 - DNA, 29 bp, Artificial Sequence
  synthetic
gcaggagattggtggacctcgctcttata

SEQ ID NO: 96 - DNA, 29 bp, Artificial Sequence
  synthetic sequence
gctggaatcagcaagtaactcagatggcc